SEQUENCE LISTING (109 sequences)

SEQ ID NO: 1 - DNA, 21 bp, Artificial Sequence
  Synthetic
cccttgagagcggtggtaccc

SEQ ID NO: 2 - DNA, 27 bp, Artificial Sequence
  Synthetic
ccctgaaggtacccgtgttgaaatcgc

SEQ ID NO: 3 - DNA, 28 bp, Artificial Sequence
  Synthetic
gcgatttcaacacgggtaccttcagggc

SEQ ID NO: 4 - DNA, 27 bp, Artificial Sequence
  Synthetic
cgggtaccttcagggcatccttagccg

SEQ ID NO: 5 - DNA, 8530 bp, Artificial Sequence
  Synthetic
gaaagggggtggtaggggccgtacggtcatgccgtgcggttccgccacccctagggggccacacggtcctgccgtgtggttcccgctggttgtacagtgacgcattgggggccgtacggtcctgccgtgcggtttcctttgcttgctgtgcaatcggggatgacaccccctttcaacgtgggtactacgaaagtgcccctcgctccgaggttaaaggagaacccccccttcttacccccactcagctcgcccttcagtgcgggcgctagcctttccacttgcagcttctgcttgtagatgcttgcaccgtgattggtgcgcttcttgctttagtcgcttgtgcttctatcgttctgacgattcagtttcctaacgccagtgtttcgacggcccaagggggtagttgcggttagtattcctaccgcaattatccctttccccgttcgtagctggtttggatcttggatctctctccttccttcccccgtcttcaatttagcttcgtgattgaagcatctcactgtctctagtatttatgtcggactgacgattgagtacgttcagattgtgtttgggaggcccaagggatcgatggacaacacttcgaaagagtcacttgtccaccgctcctttcccctaccctagcaactctggatttgctcacgtggagttcgaggtctgtactttaactctgacttgcttttcttaccttgctatcttgctgacgtggattggttgtagactgattcacgttctcgttagatgctgacgtggagtacgatcgctgtacattccactactgccaattagctcccccttcccgttgctcccctctataaggagagccttctcttgcaaaggtgaagccttcacccccggtcgaagccgcttggaataagacagggttattttctcctctcctcggcgcttgcctcttctaagctgaataggttctatctattcaggcggatggtctggtccgttccttcttggacagagtgtgtatctgggttttccggatctcgaccacacactcaccagagctcaggagtgattaagtcaaggcccgatctgcggcgaaaaggaaatgaagtattttgcagctgtagcgacctctcaaggccagcggatttccccacctggtgacaggtgcctctggggccaaaagccacgtgttaatagcacccttgagagcggtggtaccccaccaccctgcaaattatggatttgacttagtaactaaaagattgacttggcatacctcaacctgagcggcggctaaggatgccctgaaggtacccgtgttgaaatcgcttcggcgaccatggatctgatcaggggccctgcctggagtggttctatcccacacagcgtagggttaaaaaacgtctaaccgccccacaaagaccccggcagggatgccggtttcctttttaccaattcttgacactatggcacaccatgacggaattccgtgtgagagctcttgccctcttgtctacgccactgctgtcaacgaccagttcgctcttcttcacctccctgagcaggagccagaggtttatccgctggaactgctcatttgtgatctggaagacgacgtattctaccctcctcccccggatcctgacccggaaccaatggattgttctgaattcgtacattcaaggccaaattctcctatggaagttgacgactcagaagtcctggaaatctgctctatggagctcgatgagcagggcgctggatcatcaaagccatcaaccaacccaaatcagtcaggaaatacaggtacaattgtttataattactatgcaaatcagtaccaaaattcagtggatttgtccggatccgcttcgagcgcttccggagcaccgactaagcccacaaatgcgcttggaagtgtgctttcagacgcaacctctgcctttgctactatggcgcctcttctcatggataatgacacagagacaatgaccaacttggctgacagagtttccacagacacgcaaggtaatacggccgtaaacactcaatcctcggtcggccgtctctgcgcttacggtgcagagcacgcaggggaagctccctcctcctgcgctgatgaacccacatcagatgtccttgcagctcagaggtattacactatcactggacttcccgaatggacttccacccaggattttcccagctttctgtatattcctctccctcatgccctttccggtgaaaacggtggtgttttcggagccactctccgcaggcattacctgtgcaaaaccggctggcgtgttcaacttcagtgcaatgcttctcagttccattgtggttgtctaggtctcttccttgttccagaatttccccgcctcaatgaccctttccggatttctacgtcttgggatgctggctcggtctggggacgggcacaaggtaatgttactacctatgccaacctctctctcgaccacatgaactactaccagatgtgtctctacccgcatcaatttctgaatcttcgcacttctacctcctgcagtgttgaggtccccttcgtcaacattgctccctccagctcgtggacccagcatgctccctggagcatcatcatcatggtgctctcccctcttcaatactcagcaggctccacttcctctctggatcttactgtctctatagaacctgtcaaacctgtctttaatggcctacgtcatgagacccttgttcctcaggctccgatcccagttacaatcagagaacatcaaggttgcttctacactactatgccagacaccaccgtgcctgtcatgggtagaacaatctcctcgccacacgattacatgaaaggtgaggtcaaagatcttgtctccattgcccaaatccccaccttcctcggcaatgtgaagaacactcacagaatgccctacatctccacttctgtgacccaacgacagctggctaagtaccaggttactcttgcttgtgcttgcatgactaacacttcacttggctctcttgctaggaatttctctcaataccgtgggtctctttcctatgtctttgttttcactggttcagcaatggctaaaggtaagtttctcatctcctacactccccccggtgctggcgagcccatctcagtggaacaggccatgcagggaacctacgccatttgggatttaggtctaaattccacgtggcagtttactgtgcccttcatctctcccactcactatcgcctcacctcctattcttctccctctataacctctgtagatggctggctcactgtttggcaactcaccggcataacagtgccggctggcgcgcctccccagtgtgacgtcctcaccctcctaggtgctggagaagacttttctttcaagattcccattcaatcaacaattccccttacagaacagggaactgataatgcagagaagggtctcgttgaagacgagacagctgagtcagactttgttgcccaccctctctccacgcctgggaatcagacccttgtggatttcttctatgaccgctctgtttgtgtcggaactatcaccgctagcaatgcagttcggccccatgagatggtccttctttcacatttgccctcgcataatggaaatccccttcgctatatcaaggcccaacccggcaatacccgcctggaaggggttgccgatataagtgccttgttctatatgccttttacctattgtaaatatgatcttgaggtcaccgcattggatctggcttcaaatgcggctaccgcctttagtttgcattatttaccaccaggtgcccctccttatgtgttttccttaaatcgtgagcttttccccgcagctcaaccccaagctgcagctcgcaatccctcagtgtttcagccctcagttgtgaccagagccatgtccctggttattccctatgcctccccgctttcagttatgcctgcggtttggtataatggctatggcacttttaataattcaggtgagaatggtcttgcacctgatgctaatcttggtaggattgtcccttgttgtaacacttcaggaagatatcttcagtttttctttcgttacaagaattttagagcttggtgtcctagaccttcctccttctacccctggccccataccaccaaagctattacagcagaacctttcccagttcttgatcttgagatgccccgtgtttctcgtgtctactgctttgggtttaaatgccaggttggcgtcctctacgccaaactctttcagctttgccctcgttccagagccctctacaatcagacttttgttaccgacatcaacacattcacttgctttaagcggtgggtgaaaggctctccctacggaggtagatctcattttacaaatgagacttactccgccagagttctcttttttgaacgcccctatggctacaagatgcagtacaggtttggatgctcccattcgaccaagaaagtctacaaggaactctcaatggaaaacgtcatggcagagttcgactttttcagtcttcaaggctttgaaaattggcttcacgcaccacttcaagaacaaggtgcggcaatttctcaccagtatgaggaaatcccagacaggaaattcgactcagctccaaatcttcccaaatgtgatagacccaaactggaaaagcctccaaagaccctctttaacttgcttaagaaagttgtttcagaagatgaattggaccctcttcaggatctctggaccctgatcaagaaattggttaaggccttcaattcaatagttgatacacttcacaagccctacttctggattgcccaaattcggaaaatcaccaaattcattgcctacacagttctcatcaaacacaacccagatgccaccacacttgcctgcgttgcagctcttgttgggacagaaatgctcgacaaccgctccatcgtggacttcatcacaaagtgtttcagatcttggtttacaacagctcccccagcaatgatggaagaacagatgcccaaaatgaaagacctaaatgactggttcactcttggcaagaacatagagtgggtcgtcaaaatgatcaaaaccctgttcaattggattacctcttggttcaagaaagaagaagagtctccacaagggaaactcaacaagcttctcctggactttgcagaaaatgcagagacaatcaaaaactttagagcaggcaaaggcgttagacagtgcacccttaaggtgtctgtagcctacatgaaaacagtctacgatttggccatgaaagtaggaaagaccaacattgcctcagcagcttcaaaattcatggaagtaaacaaccaccaccattccagactcgagcccgtcgtcgtcgtgcttcgcggcgcaccaggacaagggaaatcagttactgcccaaattttggctcaggcaatctccaaattggaaacaggaaaacaatcagtgtattcagtcccaccagatgcaaattatctagatggatatgaaaaccagcatacagtgattatggatgatctaggtcagaacccagatggaaaagactttgtcaccttctgccagatggtgtcaaccaccaacttccttcccaatatggcttccctagagaataaaggaattcccttcacctccagagtcgtgctggccacgacaaatcaccagaaatttaaccctgttaccatctctgacgctggcgccgttgatcgtcggattaccttcgacatcaccgtccacgctcgctcagaatacaggaaaggcaggaccctagattttggaaaagcaatgcaacccatcccagatcaggaaccccctctcccatgcttcaaaacacagtgccctctcctcaatggagaggctgtttgcttcacagataataggactaatgacaattacagcctcgcagacattgtttgcctggtttgtgcagaactctcccaaaagaaagagacattggatgtagcaaatgccctagtcatgcagtcaccagaaattgttatcactctagaacagatggaagaagcaatgaaaagtgttttcgaaacagcccaccaagtcaccacagaagaaagagcagaactccttcaagcaattaaggatgccctcaatcatgcccaagtaatggatgattggatgaagatttcagctacctgtttgaatgtgatgcttgtggctttcaccggctaccagctctattcagcctggtcttcaaattctcaggaaaagcccctcaaagttgtcattgatgcagccaccgtcccaggtgaagaagaagcagcttacaatggaaaggttaagaagaagaagacagagttgatcccaatgcagctagaagccccagcaatgtccccagattttgccaactatgttctcaagaaagtagtggcacccatgacccttcgctttgaaggcggaggtgaattgacccagtcctgtctgatgattcgagatcgaatcatcgtttccaacaaacatgccctctccctagattggacacatatcaaggttaaaggactttggcacacccgtgaatccgtcaccattcaggcaatttgcaagggcggaaacacaacagacattgcagctgtgcgcctcccagcaggcgatcagtttaaggataatgttcataaattcatctcaaagaatgacccattcccaattcccatgactcagatcaccggagtcaagaatgcagatacagcaacactttacacaggtacatttgtaaaggcccagactcagattttctcaacagcaggcaatcagtacggcaatgcattccattacagagcaaacacctttaaaggctattgtggctcagcaatttttggaaaatgtggaaattcagacaaaataattggctttcactctgcaggcgcctccggcgttgcagcagggagcattctcacccgtgagatgctggaacaaatttgtgcaaatctaggaccaacccccctggaagaacaaggtgctctgaccctcattggcacaggtgaagtctctcatgtcccaaggaagaccaagctcagacgctcattggcacacccacacttcaaacccaattatgatgtggcagttctctcaaaatatgattcaaggactgacaaaaatgtagatgaagtttgctttcaaaaacatacgggcaacaaagataagctccaccccatctttgggctctattttacagagtatgctcagagagttttcacacagctaggaacagataatggctgtcttaccattcaagaagcagttgatggtgttgaaggaatggatgctatggaaagggatacctctccaggcttgccccacactctctcaggaaaaagaagagaagatgtttttgattttgaaaagaaacaatttaaaagtgaagatgcagccgcctcctacaggcagatggttgctggagattattctcatgtggtctaccaaagctttctgaaagatgaaattcggcccatagaaaaagtgcaagcagcaaaaaccagattggttgatgtcccacccttcgagcattgcttgctcggaagacagtttctaggtaaatttgcagcaaagttttacaagaacccaggcacagtgcttggttcagcaattggctgtgatccagatacagattggactaaatttgcagttgccctaagccagtacaagtatgtttatgatgttgattactcaaattttgattctactcatggtacaggcatttttgaattggctatctccaaattcttcaatgttagaaatggatttgatccacgcacaggtaactacctgcgcagcctagcaacctcagtacacgcgtatgaggatgcaaggtaccagattgtaggtggactcccctcaggatgtgcagctactagtctcctcaatacagtgtttaataatgtcatcattagagcagggctagctcttacatataaaaattttgattacgatgacattgaagttttggcctacggcgacgacttgctcgttgcttcaaatttcaaaatagattttaatttggtcaaaaataacctctcaaaagaaggttacaaaattactcctgctagtaaaggtgatactttcccactagagagcactctggatgattgtgttttcttgaagagaaagtttgttaagaacgaccttgggctttacaaaccagtaatgtctgaggaagtcttgcaagctatgctttctttctacaaaccaggtaccctggcagagaagcttctgtccgtagccctacttgctgtccattctggacagaaagtttatgatcagtgctttgctccgtttcgcgaggctggcattgtgattccaggctatgacttggtgtatgatagatggcttagtcttcatcaatgaatggattggatttcggttgagcccccacccggtacaacgctttaccttagaagccactaaggtgtacgcggtcatcggggacccctcctggcctttggtttattggtgaattactagttcagttaggttttgttagttaggaaaaaaaaaaaaaaaaaaaaaaaaaa

SEQ ID NO: 6 - DNA, 23 bp, Artificial Sequence
  Synthetic
agaagccccagcaatgtccccag

SEQ ID NO: 7 - DNA, 24 bp, Artificial Sequence
  Synthetic
ccgcccttgcaaattgcctgaatg

SEQ ID NO: 8 - protein, 110 aa, Artificial Sequence
  Synthetic
MPVSFLPILDTMAHHDGIPCESSCPLVYATAAVNDQFALLHLPEQEPEVYPLELLIDLEDDVFYPPPPPDPDPEPMDCSEFVHSRPNSPMEVDDSEVLEICSMELDEQGA

SEQ ID NO: 9 - protein, 78 aa, Artificial Sequence
  Synthetic
MMACIHGYPSVCPICTAIDDKSSDGMYLLLADNEWFPADLLTMDLDDDVFWPNDKSNVSETMDWTDLPFILDTVMEPQ

SEQ ID NO: 10 - protein, 72 aa, Artificial Sequence
  Synthetic
MACKHGYPFLCPLCTAIDDISADGSFALLFDNEWYPTDLLTVDLDDDVFHPPDCVMEWTDLPLIQDVLMEPQ

SEQ ID NO: 11 - protein, 77 aa, Artificial Sequence
  Synthetic
MACKHGYPDVCPICTAVDDATPDFEWLLMADGEWFPTDLLCVDLDDDVFWPSDTSNQSQTMEWTDIPLICDTVMEPQ

SEQ ID NO: 12 - protein, 77 aa, Artificial Sequence
  Synthetic
MACKHGYPDVCPICTAIDDVTPGFEYLLLADGEWFPTDLLCVDLDDDVFWPSDSSTQPQTMEWTDVPLVCDTVMEPQ

SEQ ID NO: 13 - protein, 68 aa, Artificial Sequence
  Synthetic
MATTMEQETCAHSLTFEECPKCSALQQYRNGFYLLKYDEEWYPEELLTDGEDDVFDPELDMEVVFELQ

SEQ ID NO: 14 - DNA, 30 bp, Artificial Sequence
  Synthetic
auggcacaccaugacggaauuccgugugag

SEQ ID NO: 15 - DNA, 30 bp, Artificial Sequence
  Synthetic
auggcgugcatccauggauacccaagcgug

SEQ ID NO: 16 - DNA, 30 bp, Artificial Sequence
  Synthetic
auggcuugcaaacauggauacccagaugug

SEQ ID NO: 17 - DNA, 30 bp, Artificial Sequence
  Synthetic
auggccugcaaacauggauacccagaugug

SEQ ID NO: 18 - DNA, 30 bp, Artificial Sequence; identical to SEQ ID NO: 16
  Synthetic

SEQ ID NO: 19 - DNA, 30 bp, Artificial Sequence; identical to SEQ ID NO: 17
  Synthetic

SEQ ID NO: 20 - DNA, 30 bp, Artificial Sequence
  Synthetic
auggcuugcaaacacggauacccagacgug

SEQ ID NO: 21 - DNA, 30 bp, Artificial Sequence; identical to SEQ ID NO: 20
  Synthetic

SEQ ID NO: 22 - DNA, 30 bp, Artificial Sequence
  Synthetic
auggcgugcaagcacggauauccguuuuug

SEQ ID NO: 23 - protein, 49 aa, Artificial Sequence
  Synthetic
LLTPLPSYSPDRPGQSPDTSKAPIQWRWISSVTESGTVSNTFPTRTRQD

SEQ ID NO: 24 - protein, 45 aa, Artificial Sequence
  Synthetic
LLTPLPSFCPDSSSGPQKTKAPVQWRWVRSGGVNGANFPLMTKQD

SEQ ID NO: 25 - protein, 41 aa, Artificial Sequence
  Synthetic
LLTPLPSDRLKENEFGLDEQHRWLSFQSATSSTPPYRTKQD

SEQ ID NO: 26 - protein, 45 aa, Artificial Sequence
  Synthetic
LLTPLPSYAPDSTSGPTETQAPVQWRWLRGTSDGSTTFPLMTKQD

SEQ ID NO: 27 - protein, 42 aa, Artificial Sequence
  Synthetic
ILTPGPQFDPAYDQLRPQRLTEIWGNGNEETSKVFPLKSKQD

SEQ ID NO: 28 - protein, 30 aa, Artificial Sequence
  Synthetic
LLSHLPSHNGNPLRYIKAQPGNTRLEGVAD

SEQ ID NO: 29 - protein, 54 aa, Artificial Sequence
  Synthetic
PEFYTGTGVATSGQEPNKVFLMDTTWQEPQAAPTGFRYDGKNGFFTLNHQNYWQ

SEQ ID NO: 30 - protein, 54 aa, Artificial Sequence
  Synthetic
PEFYTGKGTKTGTMEPSDPFTMDTEWRSPQGAPTGYRYDSRTGFFATNHQNQWQ

SEQ ID NO: 31 - protein, 56 aa, Artificial Sequence
  Synthetic
PEFDTSSYSAVDDPIGEEPFKVDTTWQTGSLRGHSYEDKSTQTLRPLALNHQNYWQ

SEQ ID NO: 32 - protein, 54 aa, Artificial Sequence
  Synthetic
PEFYTGHTPTSGTTEPTTPFTMDSSWQTPQQAPVGFRYDGRNGYFALNHQNYWQ

SEQ ID NO: 33 - protein, 43 aa, Artificial Sequence
  Synthetic
PEYPTLDAFAMDNRWSKDNLPNGTRTQTNKKGPFAMDHQNFWQ

SEQ ID NO: 34 - protein, 44 aa, Artificial Sequence
  Synthetic
PEFPRLNDPFRISTSWDAGSVWGRAQGNVTTYANLSLDHMNYYQ

SEQ ID NO: 35 - protein, 171 aa, Artificial Sequence
  Synthetic
MTEFRVRALALLSTPLLSTTSSLFFTSLSRSQRFIRWNCSFVIWKTTYSTLLPRILTRNQWIVLNSYIQGQILLWKLTTQKSWKSALWSSMSRALDHQSHQPTQISQEIQVQLFIITMQISTKIQWICPDPLRALPEHRLSPQMRLEVCFQTQPLPLLLWRLFSWIMTQRQ

SEQ ID NO: 36 - protein, 157 aa, Artificial Sequence
  Synthetic
MDTQMCALFAQPLTLLPDLNICSWQTVNGSQRTFFVWTWTMTSSGLRTRAINLKQWNGLTYRSYAILSWNPRETPLHLTRVTPSPQVTKGSLSTTSIPIINTKIQLICLPAVAMLATPPKTTDNCRTSWAALQMLLLLWHLSSWIKTQRRWRISLTE

SEQ ID NO: 37 - protein, 157 aa, Artificial Sequence
  Synthetic
MDTQACVLFAQPLTKVPTECICSWQITNGSQRIFLLWTWMMTSSGLMTRAMCLRQWTGLTFRSYSILSWNPRETPRHLTRVTPSLQAMKELLLTTSIPIISTKIQLTSLPTVETPAALLKQKDNWGTYLVMLQMHFPLWLLYFLTKIQRKWKIFQIA

SEQ ID NO: 38 - protein, 157 aa, Artificial Sequence
  Synthetic
MDTQTCALFAQPLTLLPALNICSWETENGSQRTFFVWTWTMTSSGLRTRAINLKQWNGLTYRSYAIKSWNPRETPRHLTRVTPSPQEMKGLLLITSIPIINTKIQLICLPMEATLATVPRLKDNFPTSWAALLMPLLLWHLSSWMKTQRRWKISLTE

SEQ ID NO: 39 - protein, 157 aa, Artificial Sequence
  Synthetic
TDTQTCALFAQPLTLLPTLNICSWQTENGSLRTFFVWTWTMTSSGLRTRALNLKQWNGLMYRSYAILSWNPREMPRHLIRVTPSPQEMRGLSLITSIPIINTRTQLICLPVVATLAMLPRTMDNCPAFWVELQMLLLLWHLSSWTRTQRRWKTSLTE

SEQ ID NO: 40 - protein, 57 aa, Artificial Sequence
  Synthetic
TDIRFCALFALLLTSLQMDLLLYYLTMNGTRLTSLLLTWTTTCFIPRIVLWNGLIYH

SEQ ID NO: 41 - protein, 34 aa, Artificial Sequence
  Synthetic
TDIRLCALFALLSTTLRTDFSPFCSIMNGTQLTY

SEQ ID NO: 42 - DNA, 1451 bp, Artificial Sequence
  Synthetic
gaaagggggtggtaggggccgtacggtcatgccgtgcggttccgccacccctagggggccacacggtcctgccgtgtggttcccgctggttgtacagtgacgcattgggggccgtacggtcctgccgtgcggtttcctttgcttgctgtgcaatcggggatgacaccccctttcaacgtgggtactacgaaagtgcccctcgctccgaggttaaaggagaacccccccttcttacccccactcagctcgcccttcagtgcgggcgctagcctttccacttgcagcttctgcttgtagatgcttgcaccgtgattggtgcgcttcttgctttagtcgcttgtgcttctatcgttctgacgattcagtttcctaacgccagtgtttcgacggcccaagggggtagttgcggttagtattcctaccgcaattatccctttccccgttcgtagctggtttggatcttggatctctctccttccttcccccgtcttcaatttagcttcgtgattgaagcatctcactgtctctagtatttatgtcggactgacgattgagtacgttcagattgtgtttgggaggcccaagggatcgatggacaacacttcgaaagagtcacttgtccaccgctcctttcccctaccctagcaactctggatttgctcacgtggagttcgaggtctgtactttaactctgacttgcttttcttaccttgctatcttgctgacgtggattggttgtagactgattcacgttctcgttagatgctgacgtggagtacgatcgctgtacattccactactgccaattagctcccccttcccgttgctcccctctataaggagagccttctcttgcaaaggtgaagccttcacccccggtcgaagccgcttggaataagacagggttattttctcctctcctcggcgcttgcctcttctaagctgaataggttctatctattcaggcggatggtctggtccgttccttcttggacagagtgtgtatctgggttttccggatctcgaccacacactcaccagagctcaggagtgattaagtcaaggcccgatctgcggcgaaaaggaaatgaagtattttgcagctgtagcgacctctcaaggccagcggatttccccacctggtgacaggtgcctctggggccaaaagccacgtgttaatagcacccttgagagcggtggtaccccaccaccctgcaaattatggatttgacttagtaactaaaagattgacttggcatacctcaacctgagcggcggctaaggatgccctgaaggtacccgtgttgaaatcgcttcggcgaccatggatctgatcaggggccctgcctggagtggttctatcccacacagcgtagggttaaaaaacgtctaaccgccccacaaagaccccggcagggatgccggtttcctttttaccaattcttgacact

SEQ ID NO: 43 - DNA, 294 bp, Artificial Sequence
  Synthetic
atggcacaccatgacggaattccgtgtgagagctcttgccctcttgtctacgccactgctgtcaacgaccagttcgctcttcttcacctccctgagcaggagccagaggtttatccgctggaactgctcatttgtgatctggaagacgacgtattctaccctcctcccccggatcctgacccggaaccaatggattgttctgaattcgtacattcaaggccaaattctcctatggaagttgacgactcagaagtcctggaaatctgctctatggagctcgatgagcagggcgct

SEQ ID NO: 44 - DNA, 516 bp, Artificial Sequence
  Synthetic
atgacggaattccgtgtgagagctcttgccctcttgtctacgccactgctgtcaacgaccagttcgctcttcttcacctccctgagcaggagccagaggtttatccgctggaactgctcatttgtgatctggaagacgacgtattctaccctcctcccccggatcctgacccggaaccaatggattgttctgaattcgtacattcaaggccaaattctcctatggaagttgacgactcagaagtcctggaaatctgctctatggagctcgatgagcagggcgctggatcatcaaagccatcaaccaacccaaatcagtcaggaaatacaggtacaattgtttataattactatgcaaatcagtaccaaaattcagtggatttgtccggatccgcttcgagcgcttccggagcaccgactaagcccacaaatgcgcttggaagtgtgctttcagacgcaacctctgcctttgctactatggcgcctcttctcatggataatgacacagagacaatga

SEQ ID NO: 45 - DNA, 210 bp, Artificial Sequence
  Synthetic
ggatcatcaaagccatcaaccaacccaaatcagtcaggaaatacaggtacaattgtttataattactatgcaaatcagtaccaaaattcagtggatttgtccggatccgcttcgagcgcttccggagcaccgactaagcccacaaatgcgcttggaagtgtgctttcagacgcaacctctgcctttgctactatggcgcctcttctcatg

SEQ ID NO: 46 - DNA, 774 bp, Artificial Sequence
  Synthetic
gataatgacacagagacaatgaccaacttggctgacagagtttccacagacacgcaaggtaatacggccgtaaacactcaatcctcggtcggccgtctctgcgcttacggtgcagagcacgcaggggaagctccctcctcctgcgctgatgaacccacatcagatgtccttgcagctcagaggtattacactatcactggacttcccgaatggacttccacccaggattttcccagctttctgtatattcctctccctcatgccctttccggtgaaaacggtggtgttttcggagccactctccgcaggcattacctgtgcaaaaccggctggcgtgttcaacttcagtgcaatgcttctcagttccattgtggttgtctaggtctcttccttgttccagaatttccccgcctcaatgaccctttccggatttctacgtcttgggatgctggctcggtctggggacgggcacaaggtaatgttactacctatgccaacctctctctcgaccacatgaactactaccagatgtgtctctacccgcatcaatttctgaatcttcgcacttctacctcctgcagtgttgaggtccccttcgtcaacattgctccctccagctcgtggacccagcatgctccctggagcatcatcatcatggtgctctcccctcttcaatactcagcaggctccacttcctctctggatcttactgtctctatagaacctgtcaaacctgtctttaatggcctacgtcatgagacccttgttcctcag

SEQ ID NO: 47 - DNA, 690 bp, Artificial Sequence
  Synthetic
gctccgatcccagttacaatcagagaacatcaaggttgcttctacactactatgccagacaccaccgtgcctgtcatgggtagaacaatctcctcgccacacgattacatgaaaggtgaggtcaaagatcttgtctccattgcccaaatccccaccttcctcggcaatgtgaagaacactcacagaatgccctacatctccacttctgtgacccaacgacagctggctaagtaccaggttactcttgcttgtgcttgcatgactaacacttcacttggctctcttgctaggaatttctctcaataccgtgggtctctttcctatgtctttgttttcactggttcagcaatggctaaaggtaagtttctcatctcctacactccccccggtgctggcgagcccatctcagtggaacaggccatgcagggaacctacgccatttgggatttaggtctaaattccacgtggcagtttactgtgcccttcatctctcccactcactatcgcctcacctcctattcttctccctctataacctctgtagatggctggctcactgtttggcaactcaccggcataacagtgccggctggcgcgcctccccagtgtgacgtcctcaccctcctaggtgctggagaagacttttctttcaagattcccattcaatcaacaattccccttacagaacag

SEQ ID NO: 48 - DNA, 768 bp, Artificial Sequence
  Synthetic
ggaactgataatgcagagaagggtctcgttgaagacgagacagctgagtcagactttgttgcccaccctctctccacgcctgggaatcagacccttgtggatttcttctatgaccgctctgtttgtgtcggaactatcaccgctagcaatgcagttcggccccatgagatggtccttctttcacatttgccctcgcataatggaaatccccttcgctatatcaaggcccaacccggcaatacccgcctggaaggggttgccgatataagtgccttgttctatatgccttttacctattgtaaatatgatcttgaggtcaccgcattggatctggcttcaaatgcggctaccgcctttagtttgcattatttaccaccaggtgcccctccttatgtgttttccttaaatcgtgagcttttccccgcagctcaaccccaagctgcagctcgcaatccctcagtgtttcagccctcagttgtgaccagagccatgtccctggttattccctatgcctccccgctttcagttatgcctgcggtttggtataatggctatggcacttttaataattcaggtgagaatggtcttgcacctgatgctaatcttggtaggattgtcccttgttgtaacacttcaggaagatatcttcagtttttctttcgttacaagaattttagagcttggtgtcctagaccttcctccttctacccctggccccataccaccaaagctattacagcagaacctttcccagttcttgatcttgag

SEQ ID NO: 49 - DNA, 519 bp, Artificial Sequence
  Synthetic
atgccccgtgtttctcgtgtctactgctttgggtttaaatgccaggttggcgtcctctacgccaaactctttcagctttgccctcgttccagagccctctacaatcagacttttgttaccgacatcaacacattcacttgctttaagcggtgggtgaaaggctctccctacggaggtagatctcattttacaaatgagacttactccgccagagttctcttttttgaacgcccctatggctacaagatgcagtacaggtttggatgctcccattcgaccaagaaagtctacaaggaactctcaatggaaaacgtcatggcagagttcgactttttcagtcttcaaggctttgaaaattggcttcacgcaccacttcaagaacaaggtgcggcaatttctcaccagtatgaggaaatcccagacaggaaattcgactcagctccaaatcttcccaaatgtgatagacccaaactggaaaagcctccaaagaccctctttaacttgcttaagaaagttgtt

SEQ ID NO: 50 - DNA, 306 bp, Artificial Sequence
  Synthetic
tcagaagatgaattggaccctcttcaggatctctggaccctgatcaagaaattggttaaggccttcaattcaatagttgatacacttcacaagccctacttctggattgcccaaattcggaaaatcaccaaattcattgcctacacagttctcatcaaacacaacccagatgccaccacacttgcctgcgttgcagctcttgttgggacagaaatgctcgacaaccgctccatcgtggacttcatcacaaagtgtttcagatcttggtttacaacagctcccccagcaatgatggaagaacagatg

SEQ ID NO: 51 - DNA, 978 bp, Artificial Sequence
  Synthetic
cccaaaatgaaagacctaaatgactggttcactcttggcaagaacatagagtgggtcgtcaaaatgatcaaaaccctgttcaattggattacctcttggttcaagaaagaagaagagtctccacaagggaaactcaacaagcttctcctggactttgcagaaaatgcagagacaatcaaaaactttagagcaggcaaaggcgttagacagtgcacccttaaggtgtctgtagcctacatgaaaacagtctacgatttggccatgaaagtaggaaagaccaacattgcctcagcagcttcaaaattcatggaagtaaacaaccaccaccattccagactcgagcccgtcgtcgtcgtgcttcgcggcgcaccaggacaagggaaatcagttactgcccaaattttggctcaggcaatctccaaattggaaacaggaaaacaatcagtgtattcagtcccaccagatgcaaattatctagatggatatgaaaaccagcatacagtgattatggatgatctaggtcagaacccagatggaaaagactttgtcaccttctgccagatggtgtcaaccaccaacttccttcccaatatggcttccctagagaataaaggaattcccttcacctccagagtcgtgctggccacgacaaatcaccagaaatttaaccctgttaccatctctgacgctggcgccgttgatcgtcggattaccttcgacatcaccgtccacgctcgctcagaatacaggaaaggcaggaccctagattttggaaaagcaatgcaacccatcccagatcaggaaccccctctcccatgcttcaaaacacagtgccctctcctcaatggagaggctgtttgcttcacagataataggactaatgacaattacagcctcgcagacattgtttgcctggtttgtgcagaactctcccaaaagaaagagacattggatgtagcaaatgccctagtcatgcag

SEQ ID NO: 52 - DNA, 267 bp, Artificial Sequence
  Synthetic
tcaccagaaattgttatcactctagaacagatggaagaagcaatgaaaagtgttttcgaaacagcccaccaagtcaccacagaagaaagagcagaactccttcaagcaattaaggatgccctcaatcatgcccaagtaatggatgattggatgaagatttcagctacctgtttgaatgtgatgcttgtggctttcaccggctaccagctctattcagcctggtcttcaaattctcaggaaaagcccctcaaagttgtcattgatgca

SEQ ID NO: 53 - DNA, 60 bp, Artificial Sequence
  Synthetic
gccaccgtcccaggtgaagaagaagcagcttacaatggaaaggttaagaagaagaagaca

SEQ ID NO: 54 - DNA, 657 bp, Artificial Sequence
  Synthetic
gagttgatcccaatgcagctagaagccccagcaatgtccccagattttgccaactatgttctcaagaaagtagtggcacccatgacccttcgctttgaaggcggaggtgaattgacccagtcctgtctgatgattcgagatcgaatcatcgtttccaacaaacatgccctctccctagattggacacatatcaaggttaaaggactttggcacacccgtgaatccgtcaccattcaggcaatttgcaagggcggaaacacaacagacattgcagctgtgcgcctcccagcaggcgatcagtttaaggataatgttcataaattcatctcaaagaatgacccattcccaattcccatgactcagatcaccggagtcaagaatgcagatacagcaacactttacacaggtacatttgtaaaggcccagactcagattttctcaacagcaggcaatcagtacggcaatgcattccattacagagcaaacacctttaaaggctattgtggctcagcaatttttggaaaatgtggaaattcagacaaaataattggctttcactctgcaggcgcctccggcgttgcagcagggagcattctcacccgtgagatgctggaacaaatttgtgcaaatctaggaccaacccccctggaagaacaa

SEQ ID NO: 55 - DNA, 1389 bp, Artificial Sequence
  Synthetic
ggtgctctgaccctcattggcacaggtgaagtctctcatgtcccaaggaagaccaagctcagacgctcattggcacacccacacttcaaacccaattatgatgtggcagttctctcaaaatatgattcaaggactgacaaaaatgtagatgaagtttgctttcaaaaacatacgggcaacaaagataagctccaccccatctttgggctctattttacagagtatgctcagagagttttcacacagctaggaacagataatggctgtcttaccattcaagaagcagttgatggtgttgaaggaatggatgctatggaaagggatacctctccaggcttgccccacactctctcaggaaaaagaagagaagatgtttttgattttgaaaagaaacaatttaaaagtgaagatgcagccgcctcctacaggcagatggttgctggagattattctcatgtggtctaccaaagctttctgaaagatgaaattcggcccatagaaaaagtgcaagcagcaaaaaccagattggttgatgtcccacccttcgagcattgcttgctcggaagacagtttctaggtaaatttgcagcaaagttttacaagaacccaggcacagtgcttggttcagcaattggctgtgatccagatacagattggactaaatttgcagttgccctaagccagtacaagtatgtttatgatgttgattactcaaattttgattctactcatggtacaggcatttttgaattggctatctccaaattcttcaatgttagaaatggatttgatccacgcacaggtaactacctgcgcagcctagcaacctcagtacacgcgtatgaggatgcaaggtaccagattgtaggtggactcccctcaggatgtgcagctactagtctcctcaatacagtgtttaataatgtcatcattagagcagggctagctcttacatataaaaattttgattacgatgacattgaagttttggcctacggcgacgacttgctcgttgcttcaaatttcaaaatagattttaatttggtcaaaaataacctctcaaaagaaggttacaaaattactcctgctagtaaaggtgatactttcccactagagagcactctggatgattgtgttttcttgaagagaaagtttgttaagaacgaccttgggctttacaaaccagtaatgtctgaggaagtcttgcaagctatgctttctttctacaaaccaggtaccctggcagagaagcttctgtccgtagccctacttgctgtccattctggacagaaagtttatgatcagtgctttgctccgtttcgcgaggctggcattgtgattccaggctatgacttggtgtatgatagatggcttagtcttcatcaatga

SEQ ID NO: 56 - DNA, 141 bp, Artificial Sequence
  Synthetic
atggattggatttcggttgagcccccacccggtacaacgctttaccttagaagccactaaggtgtacgcggtcatcggggacccctcctggcctttggtttattggtgaattactagttcagttaggttttgttagttagg

SEQ ID NO: 57 - protein, 2303 aa, Artificial Sequence
  Synthetic
MAHHDGIPCESSCPLVYATAVNDQFALLHLPEQEPEVYPLELLICDLEDDVFYPPPPDPDPEPMDCSEFVHSRPNSPMEVDDSEVLEICSMELDEQGAGSSKPSTNPNQSGNTGTIVYNYYANQYQNSVDLSGSASSASGAPTKPTNALGSVLSDATSAFATMAPLLMDNDTETMTNLADRVSTDTQGNTAVNTQSSVGRLCAYGAEHAGEAPSSCADEPTSDVLAAQRYYTITGLPEWTSTQDFPSFLYIPLPHALSGENGGVFGATLRRHYLCKTGWRVQLQCNASQFHCGCLGLFLVPEFPRLNDPFRISTSWDAGSVWGRAQGNVTTYANLSLDHMNYYQMCLYPHQFLNLRTSTSCSVEVPFVNIAPSSSWTQHAPWSIIIMVLSPLQYSAGSTSSLDLTVSIEPVKPVFNGLRHETLVPQAPIPVTIREHQGCFYTTMPDTTVPVMGRTISSPHDYMKGEVKDLVSIAQIPTFLGNVKNTHRMPYISTSVTQRQLAKYQVTLACACMTNTSLGSLARNFSQYRGSLSYVFVFTGSAMAKGKFLISYTPPGAGEPISVEQAMQGTYAIWDLGLNSTWQFTVPFISPTHYRLTSYSSPSITSVDGWLTVWQLTGITVPAGAPPQCDVLTLLGAGEDFSFKIPIQSTIPLTEQGTDNAEKGLVEDETAESDFVAHPLSTPGNQTLVDFFYDRSVCVGTITASNAVRPHEMVLLSHLPSHNGNPLRYIKAQPGNTRLEGVADISALFYMPFTYCKYDLEVTALDLASNAATAFSLHYLPPGAPPYVFSLNRELFPAAQPQAAARNPSVFQPSVVTRAMSLVIPYASPLSVMPAVWYNGYGTFNNSGENGLAPDANLGRIVPCCNTSGRYLQFFFRYKNFRAWCPRPSSFYPWPHTTKAITAEPFPVLDLEMPRVSRVYCFGFKCQVGVLYAKLFQLCPRSRALYNQTFVTDINTFTCFKRWVKGSPYGGRSHFTNETYSARVLFFERPYGYKMQYRFGCSHSTKKVYKELSMENVMAEFDFFSLQGFENWLHAPLQEQGAAISHQYEEIPDRKFDSAPNLPKCDRPKLEKPPKTLFNLLKKVVSEDELDPLQDLWTLIKKLVKAFNSIVDTLHKPYFWIAQIRKITKFIAYTVLIKHNPDATTLACVAALVGTEMLDNRSIVDFITKCFRSWFTTAPPAMMEEQMPKMKDLNDWFTLGKNIEWVVKMIKTLFNWITSWFKKEEESPQGKLNKLLLDFAENAETIKNFRAGKGVRQCTLKVSVAYMKTVYDLAMKVGKTNIASAASKFMEVNNHHHSRLEPVVVVLRGAPGQGKSVTAQILAQAISKLETGKQSVYSVPPDANYLDGYENQHTVIMDDLGQNPDGKDFVTFCQMVSTTNFLPNMASLENKGIPFTSRVVLATTNHQKFNPVTISDAGAVDRRITFDITVHARSEYRKGRTLDFGKAMQPIPDQEPPLPCFKTQCPLLNGEAVCFTDNRTNDNYSLADIVCLVCAELSQKKETLDVANALVMQSPEIVITLEQMEEAMKSVFETAHQVTTEERAELLQAIKDALNHAQVMDDWMKISATCLNVMLVAFTGYQLYSAWSSNSQEKPLKVVIDAATVPGEEEAAYNGKVKKKKTELIPMQLEAPAMSPDFANYVLKKVVAPMTLRFEGGGELTQSCLMIRDRIIVSNKHALSLDWTHIKVKGLWHTRESVTIQAICKGGNTTDIAAVRLPAGDQFKDNVHKFISKNDPFPIPMTQITGVKNADTATLYTGTFVKAQTQIFSTAGNQYGNAFHYRANTFKGYCGSAIFGKCGNSDKIIGFHSAGASGVAAGSILTREMLEQICANLGPTPLEEQGALTLIGTGEVSHVPRKTKLRRSLAHPHFKPNYDVAVLSKYDSRTDKNVDEVCFQKHTGNKDKLHPIFGLYFTEYAQRVFTQLGTDNGCLTIQEAVDGVEGMDAMERDTSPGLPHTLSGKRREDVFDFEKKQFKSEDAAASYRQMVAGDYSHVVYQSFLKDEIRPIEKVQAAKTRLVDVPPFEHCLLGRQFLGKFAAKFYKNPGTVLGSAIGCDPDTDWTKFAVALSQYKYVYDVDYSNFDSTHGTGIFELAISKFFNVRNGFDPRTGNYLRSLATSVHAYEDARYQIVGGLPSGCAATSLLNTVFNNVIIRAGLALTYKNFDYDDIEVLAYGDDLLVASNFKIDFNLVKNNLSKEGYKITPASKGDTFPLESTLDDCVFLKRKFVKNDLGLYKPVMSEEVLQAMLSFYKPGTLAEKLLSVALLAVHSGQKVYDQCFAPFREAGIVIPGYDLVYDRWLSLHQ

SEQ ID NO: 58 - protein, 98 aa, Artificial Sequence
  Synthetic
MAHHDGIPCESSCPLVYATAVNDQFALLHLPEQEPEVYPLELLICDLEDDVFYPPPPDPDPEPMDCSEFVHSRPNSPMEVDDSEVLEICSMELDEQGA

SEQ ID NO: 59 - protein, 50 aa, Artificial Sequence
  Synthetic
NYYANQYQNSVDLSGSASSASGAPTKPTNALGSVLSDATSAFATMAPLLM

SEQ ID NO: 60 - protein, 258 aa, Artificial Sequence
  Synthetic
DNDTETMTNLADRVSTDTQGNTAVNTQSSVGRLCAYGAEHAGEAPSSCADEPTSDVLAAQRYYTITGLPEWTSTQDFPSFLYIPLPHALSGENGGVFGATLRRHYLCKTGWRVQLQCNASQFHCGCLGLFLVPEFPRLNDPFRISTSWDAGSVWGRAQGNVTTYANLSLDHMNYYQMCLYPHQFLNLRTSTSCSVEVPFVNIAPSSSWTQHAPWSIIIMVLSPLQYSAGSTSSLDLTVSIEPVKPVFNGLRHETLVPQ

SEQ ID NO: 61 - protein, 230 aa, Artificial Sequence
  Synthetic
APIPVTIREHQGCFYTTMPDTTVPVMGRTISSPHDYMKGEVKDLVSIAQIPTFLGNVKNTHRMPYISTSVTQRQLAKYQVTLACACMTNTSLGSLARNFSQYRGSLSYVFVFTGSAMAKGKFLISYTPPGAGEPISVEQAMQGTYAIWDLGLNSTWQFTVPFISPTHYRLTSYSSPSITSVDGWLTVWQLTGITVPAGAPPQCDVLTLLGAGEDFSFKIPIQSTIPLTEQ

SEQ ID NO: 62 - protein, 256 aa, Artificial Sequence
  Synthetic
GTDNAEKGLVEDETAESDFVAHPLSTPGNQTLVDFFYDRSVCVGTITASNAVRPHEMVLLSHLPSHNGNPLRYIKAQPGNTRLEGVADISALFYMPFTYCKYDLEVTALDLASNAATAFSLHYLPPGAPPYVFSLNRELFPAAQPQAAARNPSVFQPSVVTRAMSLVIPYASPLSVMPAVWYNGYGTFNNSGENGLAPDANLGRIVPCCNTSGRYLQFFFRYKNFRAWCPRPSSFYPWPHTTKAITAEPFPVLDLE

SEQ ID NO: 63 - protein, 173 aa, Artificial Sequence
  Synthetic
MPRVSRVYCFGFKCQVGVLYAKLFQLCPRSRALYNQTFVTDINTFTCFKRWVKGSPYGGRSHFTNETYSARVLFFERPYGYKMQYRFGCSHSTKKVYKELSMENVMAEFDFFSLQGFENWLHAPLQEQGAAISHQYEEIPDRKFDSAPNLPKCDRPKLEKPPKTLFNLLKKVV

SEQ ID NO: 64 - protein, 102 aa, Artificial Sequence
  Synthetic
SEDELDPLQDLWTLIKKLVKAFNSIVDTLHKPYFWIAQIRKITKFIAYTVLIKHNPDATTLACVAALVGTEMLDNRSIVDFITKCFRSWFTTAPPAMMEEQM

SEQ ID NO: 65 - protein, 320 aa, Artificial Sequence
  Synthetic
PKMKDLNDWFTLGKNIEWVVKMIKTLFNWITSWFKKEEESPQGKLNKLLLDFAENAETIKNFRAGKGVRQCTLKVSVAYMKTVYDLAMKVGKTNIASAASKFMEVNNHHHSRLEPVVVVLRGAPGQGKSVTAQILAQAISKLETGKQSVYSVPPDANYLDGYENQHTVIMDDLGQNPDGKDFVTFCQMVSTTNFLPNMASLENKGIPFTSRVVLATTNHQKFNPVTISDAGAVDRRITFDITVHARSEYRKGRTLDFGKAMQPIPDQEPPLPCFKTQCPLLNGEAVCFTDNRTNDNYSLADIVCLVCAELSQKKETLDVA

SEQ ID NO: 66 - protein, 109 aa, Artificial Sequence
  Synthetic
SPEIVITLEQMEEAMKSVFETAHQVTTEERAELLQAIKDALNHAQVMDDWMKISATCLNVMLVAFTGYQLYSAWSSNSQEKPLKVVIDAATVPGEEEAAYNGKVKKKKT

SEQ ID NO: 67 - protein, 219 aa, Artificial Sequence
  Synthetic
ELIPMQLEAPAMSPDFANYVLKKVVAPMTLRFEGGGELTQSCLMIRDRIIVSNKHALSLDWTHIKVKGLWHTRESVTIQAICKGGNTTDIAAVRLPAGDQFKDNVHKFISKNDPFPIPMTQITGVKNADTATLYTGTFVKAQTQIFSTAGNQYGNAFHYRANTFKGYCGSAIFGKCGNSDKIIGFHSAGASGVAAGSILTREMLEQICANLGPTPLEEQ

SEQ ID NO: 68 - protein, 462 aa, Artificial Sequence
  Synthetic
GALTLIGTGEVSHVPRKTKLRRSLAHPHFKPNYDVAVLSKYDSRTDKNVDEVCFQKHTGNKDKLHPIFGLYFTEYAQRVFTQLGTDNGCLTIQEAVDGVEGMDAMERDTSPGLPHTLSGKRREDVFDFEKKQFKSEDAAASYRQMVAGDYSHVVYQSFLKDEIRPIEKVQAAKTRLVDVPPFEHCLLGRQFLGKFAAKFYKNPGTVLGSAIGCDPDTDWTKFAVALSQYKYVYDVDYSNFDSTHGTGIFELAISKFFNVRNGFDPRTGNYLRSLATSVHAYEDARYQIVGGLPSGCAATSLLNTVFNNVIIRAGLALTYKNFDYDDIEVLAYGDDLLVASNFKIDFNLVKNNLSKEGYKITPASKGDTFPLESTLDDCVFLKRKFVKNDLGLYKPVMSEEVLQAMLSFYKPGTLAEKLLSVALLAVHSGQKVYDQCFAPFREAGIVIPGYDLVYDRWLSLHQ

SEQ ID NO: 69 - DNA, 6271 bp, Artificial Sequence
  Synthetic
ggcatacctcaacctgagcggcggctaaggatgccctgaaggtacccatgatgaaatcgctctggcgaccatggatctgattaggggccctgcctggagtggatctatcccacacagcgtagggttaaaaaacgtcgaaccgccccacaatgaccccggcagggatgccggttttctctttaccaaatctgacactatggcacaccatgacggaattccgtgtgagagctcttgccctcttgtccacgccattgctgtcgacaacgagctcgttcttcttcaactccctgagcaggagccagaggtttatccgctggcgctgctcctttgtgatttggaagacgacgtgttccactcttcttccccggatcctgacccggaaccaatggattgttctgaattcgtacattcaaggccaaattctcctatggaggttgacgacccagaagtcttggaaatctgctctatggagctcgatgagcagggcgctggatcatcaaaaccatcaaccaacccaaatcagtcaggaaatacaggtacaattgtttataattactatgcaaatcagtaccaaaattcagtggatctctccggatccgcttcgagcgcttccggagcaccgactaagcccacaaatgcgcttggaagtgtgctttcagacgcaacctctgcctttgctactatggcgcctcttctcatggataatgacacagagacgatgaccaacttggctgacagggtttccacagacacgcaaggcaacacggccgtaaacactcaatcctcggtcggccgtctctgcgcttacggygcagagcacacaggagaacccccatcttcctgtgctgatgaaccyacatcagatgtccttgcagctcagaggtactacacaataactggactccctgaatggacttctacccaggaytttcccagctttctgtayattcctcttccwcaygccctttccggtgaaacgggyggtgttttcggggcaaccctccgtagacactacctstgyaaaacyggttggcgygttcaacttcagtgcaatgcttcacagtttcaytgtggctgcttrggccttttyctggttcccgagttyccwcgcctyacyaaccctttccagatttccacaarytgggaagcaggctcggtytggggaaaagcgcaaggtgaaaccaccacctacgccaacatctcccttgaccacatgaactactaccagatgtgcctatacccacaccaattcttgaatcttcgtacttccacctcctgcagtgttgaagttccctacgtcaacatcgccccttccagttcctggacccagcatgccccctggagcatcgttataatggtgctcacccctcttcgctactcagctggttccactccctctctagatcttactgtttccattgagcctgttaaacctgtcttcaatggccttcgccacgaaactcttgttacccaggcccctatcccagtaacaatcagagaacatcaaggttgcttcttcactaccatgccggacaccaccgtgcccatcatgggaagaacaattgcttcaccccatgactacatgaaaggtgaggtcaaagaccttgtttccattgcccagattcccaccttcctgggcaatgtcaaaaacacaaacagagtgccctacatctctacatctgatactcagacactcctggccaagtatcaggtaaccctggcttgtgcttgcatgaccaacacttcgcttggtgctcttgctcgcaatttttctcagtatcgtggatctctctcttatgtgtttgtttttactggttctgctatggcaaagggtaagtttcttatttcatacacccccccaggtgcaggtgaacccaccacagtagagcaagcaatgcagggaacctacgccatctgggacctcggtctcaattctacttggcaatttacagttcctttcatttcccccacccactatcgtctcacatcctattcctctccttccattacttcagttgacggatggcttaccgtttggcaactcacgggaatcaccgttccggctggagctcctccgcaatgtgatgtgttaacccttcttggtgctggagaagacttctccctcaagatccccatccaggcatatattcctcttactgaacagggtgtagataatgcagagaaaggtgtagtttcagatgagaccgcagagtcggactttgtggcccaccccgtttcctctcccggaaatcagactttggttgacttcttctatgaccgagctgtttgtgttggtgaccttgtcgctaacgttgcactcagacccgtgaaccctgcccttctttctcaccttccttctcttaatggagtgccctcacgcttcattaattcgcagtcaggcaaccaacgtgttgcgggtgttgcagatattgcctctctcttttatatgccttttacatattgtaaatatgatttggaagttactgcaatagatgtaagtggagccggtaatccaggctttggtctccactatctccccccaggtgctccacagtacattttctcggctgatcgaggtctgctgtccacactgcagccccaagcagcctcgagraatccctacatcattcagcctcagggcaatgtgagatctctytcttgygttgttccctatgcttcccccctttcagttcttcccgctgtttggtayaatggctatgcractttcaccaattctggccaaccaggcattgcycccgatgccaatcttggtcttcttgttgctagctccaaycagaatggcaagacycttcagcttttcttccgctayaaaaatttyagaggctggtgtccycgaccctcggccttcttcccctggccccayrccactcgcagtaagattgtyacacaggarccctttccagctcttgaacttgaaatgccccggatttctcgtgtctactgctttgagtttaagtgtcaggttggcattctctatgccaaactytttcagctttgccctcgttccagagccctctattctcagacctttgttactgatttcaattcattcacaagcttcaagcggtgggtgaagggttctccctatggaggcggatctccttttacaaacgagatctactccgccagagttctcttttttgaacgcccctacggctacaaaatgcagtacaggtttggatgctccctttcgaccaagaaagtatacaaggaacttacaatggaaaatgttatggcagagtttgatttcttcagtcttcaaggttttgacaattggcttcacacacccatggaagagcaaggtgcagcaatttcacaccagtatgaagaaattccagacaggaaattcgatacagctccaaatccacccaaatgcgatagacccagattggaaaagcccccgaagactctctttaatttgcttaagaaggttgtttcagaagatgaattggacccccttcaggacctctggtgcctagtcaaraagctagtaaaggcyttcaattcaatagttgatacacttcataagccctatttttggattgcccaaattcggaaaataaccaaatttatagcctacacagttctcatcaaacacaayccagatgcyaccacacttgcctgcgttgcagctcttgttgggacagaaatgctcgacaatcgctccatcgtggattttattacaaagtgcttcaagtcttggtttacaacgcctcccccggctatgatggaagaacagatgcccaaaatgaaagaccttaatgattggttcactcttggtaagaacatagagtgggtcgtcaaaatgattaaaaccctctttaattggattacttcctggttcaagaaggaagaggagtcttcccagggaaaacttaacaaactccttcttgactttgcggaaaatgcagaaataattaaaaattttagggcaggcaaaggcgttagacagtgcacccttaaggtgtctgtagcctatatgaaatcagtctatgatttggccatgaaagtaggaaaaaccaatattgcctcggcagcttcaaaattcatggaagtgaataatcatcacagctctagacttgagcccgttgtcgtcgttctycgcggcgcaccaggacaaggaaaatcagtcactgcccagatcttggctcaggcaatctccaaattggaaacaggaaagcaatcagtgtattcagttccaccagatgcaaattatttagatggttatgaaaatcagcayacagtaatyatggatgatctaggycagaatccagatggaaaagattttgccaccttctgccaratggtgtcaaccaccaacttccttcccaayatggcttccctagaaaataaaggaatccccttyacttccagagtcgtgctggccacgacaaatcatcaaagattcaaccctgttaccatctctgaygcaggcgccgttgatcgtcggatcaccttcgacctcaccgtccacgctcgctcagaatayagaaaaggcaggaccctagattttggaaaagcaatgcaacccattccagatcaagagccccctctcccttgctttaagacacagtgccctctccttaatggagaagcggtttgcttcacagacaacaggactaatgataactacagccttgcagacattgtttgcttggtctgtgcagaactctcccaaaagaaagaaacattggacgtggcaaatgctctggttatgcaatcaccagaaattgttatcactctagaacagatggaagaagcaatgaaaagtgtctttgaaactgcccaccaagtcaccacagaagagagagcagaacttcttcaggctatcaaagatgccctcaaccatgcccaagtaatggatgattggatgaagatttcagctacctgtctgaatgtgatgcttgtggctttcaccggctaccagttctattcagcctggtcttcaaattctcaggaaaaacccctcaaagttgtcattgatgcagctaccgtcccaggtgaagaagaagcagcatacaatggaaaggtcaagaagaagaagacagagttgatcccaatgcagctagaagccccagcaatgtccccagattttgccaactatgttcttaagaaagtagtggcgcccatgacccttcgctttgagggcggaggtgagttgacccagtcttgcttgatgattcgagagcgaattatcatttccaacaagcatgccctctctttagattggactcacatcaaagtaaaaggactttggcacactcgtagttccgtcaccattcaggcaatttgcaagggcggaaatacaacagacattgcagctgtgcgcctcccatcaggcgaccagtttaaggataatgtttccaaattcatctcaaagaatgacccattcccactccccatgactcagatcaccggagtcaagaatgcagacacagcaacactttacacaggcacatttgtaaaggcccagacacagattttctcaacagcaggcaatcagtatggtaatgcttttcattataaggcaaatacttttaaagggtattgtggctcagcaatttttggaaagtgtggaaattcagacaaaataattggctttcactctgcaggcgcctctggcgttgcagcaggcagcattctcacccgtgagatgctggaacaaatttgtgcaaatctaggaccaacccccctggaagaacaaggtgctctgaccctcattggcacaggngaagtttcccatgtcccaaggaagaccaaactcaggcgctcattggcacaccctcattttaaacccaattatgatgtggcagttctttcaaaatacgattcaagaactgacaaaaatgtagatgaagtttgttttcaaaaacatacaggcaacaaggacaagctccaccccatcttcgggctgtacttcacagagtacgctcaaagagtcttcacacagctaggaacagataatagttgtctcaccatccaagaagcagttgatggngttgaaggaatggatgctatggaaaaggatacctctccnggntngcccnnnnctctttcaggaaananaagagaanatgtttttgantttgaaaagaaacagtttaaaagtgnaanacncnnccncctcctataggcaaatggntngcgggagattanttctcntgtggnctaccaaagctttttgaaagangaaatncggnccntgnnaaaagtgcaagcancaaaaaccagantngnttgatgtccctcc

SEQ ID NO: 70 - DNA, 6075 bp, Artificial Sequence
  Synthetic
atggcacaccatgacggaattccgtgtgagagctcttgccctcttgtccacgccattgctgtcgacaacgagctcgttcttcttcaactccctgagcaggagccagaggtttatccgctggcgctgctcctttgtgatttggaagacgacgtgttccactcttcttccccggatcctgacccggaaccaatggattgttctgaattcgtacattcaaggccaaattctcctatggaggttgacgacccagaagtcttggaaatctgctctatggagctcgatgagcagggcgctggatcatcaaaaccatcaaccaacccaaatcagtcaggaaatacaggtacaattgtttataattactatgcaaatcagtaccaaaattcagtggatctctccggatccgcttcgagcgcttccggagcaccgactaagcccacaaatgcgcttggaagtgtgctttcagacgcaacctctgcctttgctactatggcgcctcttctcatggataatgacacagagacgatgaccaacttggctgacagggtttccacagacacgcaaggcaacacggccgtaaacactcaatcctcggtcggccgtctctgcgcttacggygcagagcacacaggagaacccccatcttcctgtgctgatgaaccyacatcagatgtccttgcagctcagaggtactacacaataactggactccctgaatggacttctacccaggaytttcccagctttctgtayattcctcttccwcaygccctttccggtgaaacgggyggtgttttcggggcaaccctccgtagacactacctstgyaaaacyggttggcgygttcaacttcagtgcaatgcttcacagtttcaytgtggctgcttrggccttttyctggttcccgagttyccwcgcctyacyaaccctttccagatttccacaarytgggaagcaggctcggtytggggaaaagcgcaaggtgaaaccaccacctacgccaacatctcccttgaccacatgaactactaccagatgtgcctatacccacaccaattcttgaatcttcgtacttccacctcctgcagtgttgaagttccctacgtcaacatcgccccttccagttcctggacccagcatgccccctggagcatcgttataatggtgctcacccctcttcgctactcagctggttccactccctctctagatcttactgtttccattgagcctgttaaacctgtcttcaatggccttcgccacgaaactcttgttacccaggcccctatcccagtaacaatcagagaacatcaaggttgcttcttcactaccatgccggacaccaccgtgcccatcatgggaagaacaattgcttcaccccatgactacatgaaaggtgaggtcaaagaccttgtttccattgcccagattcccaccttcctgggcaatgtcaaaaacacaaacagagtgccctacatctctacatctgatactcagacactcctggccaagtatcaggtaaccctggcttgtgcttgcatgaccaacacttcgcttggtgctcttgctcgcaatttttctcagtatcgtggatctctctcttatgtgtttgtttttactggttctgctatggcaaagggtaagtttcttatttcatacacccccccaggtgcaggtgaacccaccacagtagagcaagcaatgcagggaacctacgccatctgggacctcggtctcaattctacttggcaatttacagttcctttcatttcccccacccactatcgtctcacatcctattcctctccttccattacttcagttgacggatggcttaccgtttggcaactcacgggaatcaccgttccggctggagctcctccgcaatgtgatgtgttaacccttcttggtgctggagaagacttctccctcaagatccccatccaggcatatattcctcttactgaacagggtgtagataatgcagagaaaggtgtagtttcagatgagaccgcagagtcggactttgtggcccaccccgtttcctctcccggaaatcagactttggttgacttcttctatgaccgagctgtttgtgttggtgaccttgtcgctaacgttgcactcagacccgtgaaccctgcccttctttctcaccttccttctcttaatggagtgccctcacgcttcattaattcgcagtcaggcaaccaacgtgttgcgggtgttgcagatattgcctctctcttttatatgccttttacatattgtaaatatgatttggaagttactgcaatagatgtaagtggagccggtaatccaggctttggtctccactatctccccccaggtgctccacagtacattttctcggctgatcgaggtctgctgtccacactgcagccccaagcagcctcgagraatccctacatcattcagcctcagggcaatgtgagatctctytcttgygttgttccctatgcttcccccctttcagttcttcccgctgtttggtayaatggctatgcractttcaccaattctggccaaccaggcattgcycccgatgccaatcttggtcttcttgttgctagctccaaycagaatggcaagacycttcagcttttcttccgctayaaaaatttyagaggctggtgtccycgaccctcggccttcttcccctggccccayrccactcgcagtaagattgtyacacaggarccctttccagctcttgaacttgaaatgccccggatttctcgtgtctactgctttgagtttaagtgtcaggttggcattctctatgccaaactytttcagctttgccctcgttccagagccctctattctcagacctttgttactgatttcaattcattcacaagcttcaagcggtgggtgaagggttctccctatggaggcggatctccttttacaaacgagatctactccgccagagttctcttttttgaacgcccctacggctacaaaatgcagtacaggtttggatgctccctttcgaccaagaaagtatacaaggaacttacaatggaaaatgttatggcagagtttgatttcttcagtcttcaaggttttgacaattggcttcacacacccatggaagagcaaggtgcagcaatttcacaccagtatgaagaaattccagacaggaaattcgatacagctccaaatccacccaaatgcgatagacccagattggaaaagcccccgaagactctctttaatttgcttaagaaggttgtttcagaagatgaattggacccccttcaggacctctggtgcctagtcaaraagctagtaaaggcyttcaattcaatagttgatacacttcataagccctatttttggattgcccaaattcggaaaataaccaaatttatagcctacacagttctcatcaaacacaayccagatgcyaccacacttgcctgcgttgcagctcttgttgggacagaaatgctcgacaatcgctccatcgtggattttattacaaagtgcttcaagtcttggtttacaacgcctcccccggctatgatggaagaacagatgcccaaaatgaaagaccttaatgattggttcactcttggtaagaacatagagtgggtcgtcaaaatgattaaaaccctctttaattggattacttcctggttcaagaaggaagaggagtcttcccagggaaaacttaacaaactccttcttgactttgcggaaaatgcagaaataattaaaaattttagggcaggcaaaggcgttagacagtgcacccttaaggtgtctgtagcctatatgaaatcagtctatgatttggccatgaaagtaggaaaaaccaatattgcctcggcagcttcaaaattcatggaagtgaataatcatcacagctctagacttgagcccgttgtcgtcgttctycgcggcgcaccaggacaaggaaaatcagtcactgcccagatcttggctcaggcaatctccaaattggaaacaggaaagcaatcagtgtattcagttccaccagatgcaaattatttagatggttatgaaaatcagcayacagtaatyatggatgatctaggycagaatccagatggaaaagattttgccaccttctgccaratggtgtcaaccaccaacttccttcccaayatggcttccctagaaaataaaggaatccccttyacttccagagtcgtgctggccacgacaaatcatcaaagattcaaccctgttaccatctctgaygcaggcgccgttgatcgtcggatcaccttcgacctcaccgtccacgctcgctcagaatayagaaaaggcaggaccctagattttggaaaagcaatgcaacccattccagatcaagagccccctctcccttgctttaagacacagtgccctctccttaatggagaagcggtttgcttcacagacaacaggactaatgataactacagccttgcagacattgtttgcttggtctgtgcagaactctcccaaaagaaagaaacattggacgtggcaaatgctctggttatgcaatcaccagaaattgttatcactctagaacagatggaagaagcaatgaaaagtgtctttgaaactgcccaccaagtcaccacagaagagagagcagaacttcttcaggctatcaaagatgccctcaaccatgcccaagtaatggatgattggatgaagatttcagctacctgtctgaatgtgatgcttgtggctttcaccggctaccagttctattcagcctggtcttcaaattctcaggaaaaacccctcaaagttgtcattgatgcagctaccgtcccaggtgaagaagaagcagcatacaatggaaaggtcaagaagaagaagacagagttgatcccaatgcagctagaagccccagcaatgtccccagattttgccaactatgttcttaagaaagtagtggcgcccatgacccttcgctttgagggcggaggtgagttgacccagtcttgcttgatgattcgagagcgaattatcatttccaacaagcatgccctctctttagattggactcacatcaaagtaaaaggactttggcacactcgtagttccgtcaccattcaggcaatttgcaagggcggaaatacaacagacattgcagctgtgcgcctcccatcaggcgaccagtttaaggataatgtttccaaattcatctcaaagaatgacccattcccactccccatgactcagatcaccggagtcaagaatgcagacacagcaacactttacacaggcacatttgtaaaggcccagacacagattttctcaacagcaggcaatcagtatggtaatgcttttcattataaggcaaatacttttaaagggtattgtggctcagcaatttttggaaagtgtggaaattcagacaaaataattggctttcactctgcaggcgcctctggcgttgcagcaggcagcattctcacccgtgagatgctggaacaaatttgtgcaaatctaggaccaacccccctggaagaacaaggtgctctgaccctcattggcacaggngaagtttcccatgtcccaaggaagaccaaactcaggcgctcattggcacaccctcattttaaacccaattatgatgtggcagttctttcaaaatacgattcaagaactgacaaaaatgtagatgaagtttgttttcaaaaacatacaggcaacaaggacaagctccaccccatcttcgggctgtacttcacagagtacgctcaaagagtcttcacacagctaggaacagataatagttgtctcaccatccaagaagcagttgatggngttgaaggaatggatgctatggaaaaggatacctctccnggntngcccnnnnctctttcaggaaananaagagaanatgtttttgantttgaaaagaaacagtttaaaagtgnaanacncnnccncctcctataggcaaatggntngcgggagattanttctcntgtggnctaccaaagctttttgaaagangaaatncggnccntgnnaaaagtgcaagcancaaaaaccagantngnttgatgtccctcc

SEQ ID NO: 71 - DNA, 294 bp, Artificial Sequence
  Synthetic
atggcacaccatgacggaattccgtgtgagagctcttgccctcttgtccacgccattgctgtcgacaacgagctcgttcttcttcaactccctgagcaggagccagaggtttatccgctggcgctgctcctttgtgatttggaagacgacgtgttccactcttcttccccggatcctgacccggaaccaatggattgttctgaattcgtacattcaaggccaaattctcctatggaggttgacgacccagaagtcttggaaatctgctctatggagctcgatgagcagggcgct

SEQ ID NO: 72 - DNA, 516 bp, Artificial Sequence
  Synthetic
atgacggaattccgtgtgagagctcttgccctcttgtccacgccattgctgtcgacaacgagctcgttcttcttcaactccctgagcaggagccagaggtttatccgctggcgctgctcctttgtgatttggaagacgacgtgttccactcttcttccccggatcctgacccggaaccaatggattgttctgaattcgtacattcaaggccaaattctcctatggaggttgacgacccagaagtcttggaaatctgctctatggagctcgatgagcagggcgctggatcatcaaaaccatcaaccaacccaaatcagtcaggaaatacaggtacaattgtttataattactatgcaaatcagtaccaaaattcagtggatctctccggatccgcttcgagcgcttccggagcaccgactaagcccacaaatgcgcttggaagtgtgctttcagacgcaacctctgcctttgctactatggcgcctcttctcatggataatgacacagagacgatga

SEQ ID NO: 73 - DNA, 210 bp, Artificial Sequence
  Synthetic
ggatcatcaaaaccatcaaccaacccaaatcagtcaggaaatacaggtacaattgtttataattactatgcaaatcagtaccaaaattcagtggatctctccggatccgcttcgagcgcttccggagcaccgactaagcccacaaatgcgcttggaagtgtgctttcagacgcaacctctgcctttgctactatggcgcctcttctcatg

SEQ ID NO: 74 - DNA, 774 bp, Artificial Sequence
  Synthetic
gataatgacacagagacgatgaccaacttggctgacagggtttccacagacacgcaaggcaacacggccgtaaacactcaatcctcggtcggccgtctctgcgcttacggygcagagcacacaggagaacccccatcttcctgtgctgatgaaccyacatcagatgtccttgcagctcagaggtactacacaataactggactccctgaatggacttctacccaggaytttcccagctttctgtayattcctcttccwcaygccctttccggtgaaacgggyggtgttttcggggcaaccctccgtagacactacctstgyaaaacyggttggcgygttcaacttcagtgcaatgcttcacagtttcaytgtggctgcttrggccttttyctggttcccgagttyccwcgcctyacyaaccctttccagatttccacaarytgggaagcaggctcggtytggggaaaagcgcaaggtgaaaccaccacctacgccaacatctcccttgaccacatgaactactaccagatgtgcctatacccacaccaattcttgaatcttcgtacttccacctcctgcagtgttgaagttccctacgtcaacatcgccccttccagttcctggacccagcatgccccctggagcatcgttataatggtgctcacccctcttcgctactcagctggttccactccctctctagatcttactgtttccattgagcctgttaaacctgtcttcaatggccttcgccacgaaactcttgttacccag

SEQ ID NO: 75 - DNA, 690 bp, Artificial Sequence
  Synthetic
gcccctatcccagtaacaatcagagaacatcaaggttgcttcttcactaccatgccggacaccaccgtgcccatcatgggaagaacaattgcttcaccccatgactacatgaaaggtgaggtcaaagaccttgtttccattgcccagattcccaccttcctgggcaatgtcaaaaacacaaacagagtgccctacatctctacatctgatactcagacactcctggccaagtatcaggtaaccctggcttgtgcttgcatgaccaacacttcgcttggtgctcttgctcgcaatttttctcagtatcgtggatctctctcttatgtgtttgtttttactggttctgctatggcaaagggtaagtttcttatttcatacacccccccaggtgcaggtgaacccaccacagtagagcaagcaatgcagggaacctacgccatctgggacctcggtctcaattctacttggcaatttacagttcctttcatttcccccacccactatcgtctcacatcctattcctctccttccattacttcagttgacggatggcttaccgtttggcaactcacgggaatcaccgttccggctggagctcctccgcaatgtgatgtgttaacccttcttggtgctggagaagacttctccctcaagatccccatccaggcatatattcctcttactgaacag

SEQ ID NO: 76 - DNA, 774 bp, Artificial Sequence
  Synthetic
ggtgtagataatgcagagaaaggtgtagtttcagatgagaccgcagagtcggactttgtggcccaccccgtttcctctcccggaaatcagactttggttgacttcttctatgaccgagctgtttgtgttggtgaccttgtcgctaacgttgcactcagacccgtgaaccctgcccttctttctcaccttccttctcttaatggagtgccctcacgcttcattaattcgcagtcaggcaaccaacgtgttgcgggtgttgcagatattgcctctctcttttatatgccttttacatattgtaaatatgatttggaagttactgcaatagatgtaagtggagccggtaatccaggctttggtctccactatctccccccaggtgctccacagtacattttctcggctgatcgaggtctgctgtccacactgcagccccaagcagcctcgagraatccctacatcattcagcctcagggcaatgtgagatctctytcttgygttgttccctatgcttcccccctttcagttcttcccgctgtttggtayaatggctatgcractttcaccaattctggccaaccaggcattgcycccgatgccaatcttggtcttcttgttgctagctccaaycagaatggcaagacycttcagcttttcttccgctayaaaaatttyagaggctggtgtccycgaccctcggccttcttcccctggccccayrccactcgcagtaagattgtyacacaggarccctttccagctcttgaacttgaa

SEQ ID NO: 77 - DNA, 519 bp, Artificial Sequence
  Synthetic
atgccccggatttctcgtgtctactgctttgagtttaagtgtcaggttggcattctctatgccaaactytttcagctttgccctcgttccagagccctctattctcagacctttgttactgatttcaattcattcacaagcttcaagcggtgggtgaagggttctccctatggaggcggatctccttttacaaacgagatctactccgccagagttctcttttttgaacgcccctacggctacaaaatgcagtacaggtttggatgctccctttcgaccaagaaagtatacaaggaacttacaatggaaaatgttatggcagagtttgatttcttcagtcttcaaggttttgacaattggcttcacacacccatggaagagcaaggtgcagcaatttcacaccagtatgaagaaattccagacaggaaattcgatacagctccaaatccacccaaatgcgatagacccagattggaaaagcccccgaagactctctttaatttgcttaagaaggttgtt

SEQ ID NO: 78 - DNA, 306 bp, Artificial Sequence
  Synthetic
tcagaagatgaattggacccccttcaggacctctggtgcctagtcaaraagctagtaaaggcyttcaattcaatagttgatacacttcataagccctatttttggattgcccaaattcggaaaataaccaaatttatagcctacacagttctcatcaaacacaayccagatgcyaccacacttgcctgcgttgcagctcttgttgggacagaaatgctcgacaatcgctccatcgtggattttattacaaagtgcttcaagtcttggtttacaacgcctcccccggctatgatggaagaacagatg

SEQ ID NO: 79 - DNA, 978 bp, Artificial Sequence
  Synthetic
cccaaaatgaaagaccttaatgattggttcactcttggtaagaacatagagtgggtcgtcaaaatgattaaaaccctctttaattggattacttcctggttcaagaaggaagaggagtcttcccagggaaaacttaacaaactccttcttgactttgcggaaaatgcagaaataattaaaaattttagggcaggcaaaggcgttagacagtgcacccttaaggtgtctgtagcctatatgaaatcagtctatgatttggccatgaaagtaggaaaaaccaatattgcctcggcagcttcaaaattcatggaagtgaataatcatcacagctctagacttgagcccgttgtcgtcgttctycgcggcgcaccaggacaaggaaaatcagtcactgcccagatcttggctcaggcaatctccaaattggaaacaggaaagcaatcagtgtattcagttccaccagatgcaaattatttagatggttatgaaaatcagcayacagtaatyatggatgatctaggycagaatccagatggaaaagattttgccaccttctgccaratggtgtcaaccaccaacttccttcccaayatggcttccctagaaaataaaggaatccccttyacttccagagtcgtgctggccacgacaaatcatcaaagattcaaccctgttaccatctctgaygcaggcgccgttgatcgtcggatcaccttcgacctcaccgtccacgctcgctcagaatayagaaaaggcaggaccctagattttggaaaagcaatgcaacccattccagatcaagagccccctctcccttgctttaagacacagtgccctctccttaatggagaagcggtttgcttcacagacaacaggactaatgataactacagccttgcagacattgtttgcttggtctgtgcagaactctcccaaaagaaagaaacattggacgtggcaaatgctctggttatgcaa

SEQ ID NO: 80 - DNA, 267 bp, Artificial Sequence
  Synthetic
tcaccagaaattgttatcactctagaacagatggaagaagcaatgaaaagtgtctttgaaactgcccaccaagtcaccacagaagagagagcagaacttcttcaggctatcaaagatgccctcaaccatgcccaagtaatggatgattggatgaagatttcagctacctgtctgaatgtgatgcttgtggctttcaccggctaccagttctattcagcctggtcttcaaattctcaggaaaaacccctcaaagttgtcattgatgca

SEQ ID NO: 81 - DNA, 60 bp, Artificial Sequence
  Synthetic
gctaccgtcccaggtgaagaagaagcagcatacaatggaaaggtcaagaagaagaagaca

SEQ ID NO: 82 - DNA, 657 bp, Artificial Sequence
  Synthetic
gagttgatcccaatgcagctagaagccccagcaatgtccccagattttgccaactatgttcttaagaaagtagtggcgcccatgacccttcgctttgagggcggaggtgagttgacccagtcttgcttgatgattcgagagcgaattatcatttccaacaagcatgccctctctttagattggactcacatcaaagtaaaaggactttggcacactcgtagttccgtcaccattcaggcaatttgcaagggcggaaatacaacagacattgcagctgtgcgcctcccatcaggcgaccagtttaaggataatgtttccaaattcatctcaaagaatgacccattcccactccccatgactcagatcaccggagtcaagaatgcagacacagcaacactttacacaggcacatttgtaaaggcccagacacagattttctcaacagcaggcaatcagtatggtaatgcttttcattataaggcaaatacttttaaagggtattgtggctcagcaatttttggaaagtgtggaaattcagacaaaataattggctttcactctgcaggcgcctctggcgttgcagcaggcagcattctcacccgtgagatgctggaacaaatttgtgcaaatctaggaccaacccccctggaagaacaa

SEQ ID NO: 83 - DNA, 546 bp, Artificial Sequence
  Synthetic
ggtgctctgaccctcattggcacaggngaagtttcccatgtcccaaggaagaccaaactcaggcgctcattggcacaccctcattttaaacccaattatgatgtggcagttctttcaaaatacgattcaagaactgacaaaaatgtagatgaagtttgttttcaaaaacatacaggcaacaaggacaagctccaccccatcttcgggctgtacttcacagagtacgctcaaagagtcttcacacagctaggaacagataatagttgtctcaccatccaagaagcagttgatggngttgaaggaatggatgctatggaaaaggatacctctccnggntngcccnnnnctctttcaggaaananaagagaanatgtttttgantttgaaaagaaacagtttaaaagtgnaanacncnnccncctcctataggcaaatggntngcgggagattanttctcntgtggnctaccaaagctttttgaaagangaaatncggnccntgnnaaaagtgcaagcancaaaaaccagantngnttgatgtccctcc

SEQ ID NO: 84 - protein, 1978 aa, Artificial Sequence
  Synthetic
MAHHDGIPCESSCPLVHAIAVDNELVLLQLPEQEPEVYPLALLLCDLEDDVFHSSSPDPDPEPMDCSEFVHSRPNSPMEVDDPEVLEICSMELDEQGAGSSKPSTNPNQSGNTGTIVYNYYANQYQNSVDLSGSASSASGAPTKPTNALGSVLSDATSAFATMAPLLMDNDTETMTNLADRVSTDTQGNTAVNTQSSVGRLCAYGAEHTGEPPSSCADEPTSDVLAAQRYYTITGLPEWTSTQDFPSFLYIPLPHALSGETGGVFGATLRRHYLCKTGWRVQLQCNASQFHCGCLGLFLVPEFPRLTNPFQISTXWEAGSVWGKAQGETTTYANISLDHMNYYQMCLYPHQFLNLRTSTSCSVEVPYVNIAPSSSWTQHAPWSIVIMVLTPLRYSAGSTPSLDLTVSIEPVKPVFNGLRHETLVTQAPIPVTIREHQGCFFTTMPDTTVPIMGRTIASPHDYMKGEVKDLVSIAQIPTFLGNVKNTNRVPYISTSDTQTLLAKYQVTLACACMTNTSLGALARNFSQYRGSLSYVFVFTGSAMAKGKFLISYTPPGAGEPTTVEQAMQGTYAIWDLGLNSTWQFTVPFISPTHYRLTSYSSPSITSVDGWLTVWQLTGITVPAGAPPQCDVLTLLGAGEDFSLKIPIQAYIPLTEQGVDNAEKGVVSDETAESDFVAHPVSSPGNQTLVDFFYDRAVCVGDLVANVALRPVNPALLSHLPSLNGVPSRFINSQSGNQRVAGVADIASLFYMPFTYCKYDLEVTAIDVSGAGNPGFGLHYLPPGAPQYIFSADRGLLSTLQPQAASRNPYIIQPQGNVRSLSCVVPYASPLSVLPAVWYNGYATFTNSGQPGIAPDANLGLLVASSNQNGKTLQLFFRYKNFRGWCPRPSAFFPWPHXTRSKIVTQEPFPALELEMPRISRVYCFEFKCQVGILYAKLFQLCPRSRALYSQTFVTDFNSFTSFKRWVKGSPYGGGSPFTNEIYSARVLFFERPYGYKMQYRFGCSLSTKKVYKELTMENVMAEFDFFSLQGFDNWLHTPMEEQGAAISHQYEEIPDRKFDTAPNPPKCDRPRLEKPPKTLFNLLKKVVSEDELDPLQDLWCLVKKLVKAFNSIVDTLHKPYFWIAQIRKITKFIAYTVLIKHNPDATTLACVAALVGTEMLDNRSIVDFITKCFKSWFTTPPPAMMEEQMPKMKDLNDWFTLGKNIEWVVKMIKTLFNWITSWFKKEEESSQGKLNKLLLDFAENAEIIKNFRAGKGVRQCTLKVSVAYMKSVYDLAMKVGKTNIASAASKFMEVNNHHSSRLEPVVVVLRGAPGQGKSVTAQILAQAISKLETGKQSVYSVPPDANYLDGYENQHTVIMDDLGQNPDGKDFATFCQMVSTTNFLPNMASLENKGIPFTSRVVLATTNHQRFNPVTISDAGAVDRRITFDLTVHARSEYRKGRTLDFGKAMQPIPDQEPPLPCFKTQCPLLNGEAVCFTDNRTNDNYSLADIVCLVCAELSQKKETLDVANALVMQSPEIVITLEQMEEAMKSVFETAHQVTTEERAELLQAIKDALNHAQVMDDWMKISATCLNVMLVAFTGYQFYSAWSSNSQEKPLKVVIDAATVPGEEEAAYNGKVKKKKTELIPMQLEAPAMSPDFANYVLKKVVAPMTLRFEGGGELTQSCLMIRERIIISNKHALSLDWTHIKVKGLWHTRSSVTIQAICKGGNTTDIAAVRLPSGDQFKDNVSKFISKNDPFPLPMTQITGVKNADTATLYTGTFVKAQTQIFSTAGNQYGNAFHYKANTFKGYCGSAIFGKCGNSDKIIGFHSAGASGVAAGSILTREMLEQICANLGPTPLEEQGALTLIGTGEVSHVPRKTKLRRSLAHPHFKPNYDVAVLSKYDSRTDKNVDEVCFQKHTGNKDKLHPIFGLYFTEYAQRVFTQLGTDNSCLTIQEAVDGVEGMDAMEKDTSPGXPXXLSGXXREXVFXFEKKQFKS

SEQ ID NO: 85 - protein, 98 aa, Artificial Sequence
  Synthetic
MAHHDGIPCESSCPLVHAIAVDNELVLLQLPEQEPEVYPLALLLCDLEDDVFHSSSPDPDPEPMDCSEFVHSRPNSPMEVDDPEVLEICSMELDEQGA

SEQ ID NO: 86 - protein, 171 aa, Artificial Sequence
  Synthetic
MTEFRVRALALLSTPLLSTTSSFFFNSLSRSQRFIRWRCSFVIWKTTCSTLLPRILTRNQWIVLNSYIQGQILLWRLTTQKSWKSALWSSMSRALDHQNHQPTQISQEIQVQLFIITMQISTKIQWISPDPLRALPEHRLSPQMRLEVCFQTQPLPLLLWRLFSWIMTQRR

SEQ ID NO: 87 - protein, 70 aa, Artificial Sequence
  Synthetic
GSSKPSTNPNQSGNTGTIVYNYYANQYQNSVDLSGSASSASGAPTKPTNALGSVLSDATSAFATMAPLLM

SEQ ID NO: 88 - protein, 258 aa, Artificial Sequence
  Synthetic
DNDTETMTNLADRVSTDTQGNTAVNTQSSVGRLCAYGAEHTGEPPSSCADEPTSDVLAAQRYYTITGLPEWTSTQDFPSFLYIPLPHALSGETGGVFGATLRRHYLCKTGWRVQLQCNASQFHCGCLGLFLVPEFPRLTNPFQISTXWEAGSVWGKAQGETTTYANISLDHMNYYQMCLYPHQFLNLRTSTSCSVEVPYVNIAPSSSWTQHAPWSIVIMVLTPLRYSAGSTPSLDLTVSIEPVKPVFNGLRHETLVTQ

SEQ ID NO: 89 - protein, 230 aa, Artificial Sequence
  Synthetic
APIPVTIREHQGCFFTTMPDTTVPIMGRTIASPHDYMKGEVKDLVSIAQIPTFLGNVKNTNRVPYISTSDTQTLLAKYQVTLACACMTNTSLGALARNFSQYRGSLSYVFVFTGSAMAKGKFLISYTPPGAGEPTTVEQAMQGTYAIWDLGLNSTWQFTVPFISPTHYRLTSYSSPSITSVDGWLTVWQLTGITVPAGAPPQCDVLTLLGAGEDFSLKIPIQAYIPLTEQ

SEQ ID NO: 90 - protein, 258 aa, Artificial Sequence
  Synthetic
GVDNAEKGVVSDETAESDFVAHPVSSPGNQTLVDFFYDRAVCVGDLVANVALRPVNPALLSHLPSLNGVPSRFINSQSGNQRVAGVADIASLFYMPFTYCKYDLEVTAIDVSGAGNPGFGLHYLPPGAPQYIFSADRGLLSTLQPQAASRNPYIIQPQGNVRSLSCVVPYASPLSVLPAVWYNGYATFTNSGQPGIAPDANLGLLVASSNQNGKTLQLFFRYKNFRGWCPRPSAFFPWPHXTRSKIVTQEPFPALELE

SEQ ID NO: 91 - protein, 173 aa, Artificial Sequence
  Synthetic
MPRISRVYCFEFKCQVGILYAKLFQLCPRSRALYSQTFVTDFNSFTSFKRWVKGSPYGGGSPFTNEIYSARVLFFERPYGYKMQYRFGCSLSTKKVYKELTMENVMAEFDFFSLQGFDNWLHTPMEEQGAAISHQYEEIPDRKFDTAPNPPKCDRPRLEKPPKTLFNLLKKVV

SEQ ID NO: 92 - protein, 102 aa, Artificial Sequence
  Synthetic
SEDELDPLQDLWCLVKKLVKAFNSIVDTLHKPYFWIAQIRKITKFIAYTVLIKHNPDATTLACVAALVGTEMLDNRSIVDFITKCFKSWFTTPPPAMMEEQM

SEQ ID NO: 93 - protein, 326 aa, Artificial Sequence
  Synthetic
PKMKDLNDWFTLGKNIEWVVKMIKTLFNWITSWFKKEEESSQGKLNKLLLDFAENAEIIKNFRAGKGVRQCTLKVSVAYMKSVYDLAMKVGKTNIASAASKFMEVNNHHSSRLEPVVVVLRGAPGQGKSVTAQILAQAISKLETGKQSVYSVPPDANYLDGYENQHTVIMDDLGQNPDGKDFATFCQMVSTTNFLPNMASLENKGIPFTSRVVLATTNHQRFNPVTISDAGAVDRRITFDLTVHARSEYRKGRTLDFGKAMQPIPDQEPPLPCFKTQCPLLNGEAVCFTDNRTNDNYSLADIVCLVCAELSQKKETLDVANALVMQ

SEQ ID NO: 94 - protein, 109 aa, Artificial Sequence
  Synthetic
SPEIVITLEQMEEAMKSVFETAHQVTTEERAELLQAIKDALNHAQVMDDWMKISATCLNVMLVAFTGYQFYSAWSSNSQEKPLKVVIDAATVPGEEEAAYNGKVKKKKT

SEQ ID NO: 95 - protein, 219 aa, Artificial Sequence
  Synthetic
ELIPMQLEAPAMSPDFANYVLKKVVAPMTLRFEGGGELTQSCLMIRERIIISNKHALSLDWTHIKVKGLWHTRSSVTIQAICKGGNTTDIAAVRLPSGDQFKDNVSKFISKNDPFPLPMTQITGVKNADTATLYTGTFVKAQTQIFSTAGNQYGNAFHYKANTFKGYCGSAIFGKCGNSDKIIGFHSAGASGVAAGSILTREMLEQICANLGPTPLEEQ

SEQ ID NO: 96 - protein, 112 aa, Artificial Sequence
  Synthetic
GALTLIGTGEVSHVPRKTKLRRSLAHPHFKPNYDVAVLSKYDSRTDKNVDEVCFQKHTGNKDKLHPIFGLYFTEYAQRVFTQLGTDNSCLTIQEAVDGVEGMDAMEKDTSPG

SEQ ID NO: 97 - DNA, 8546 bp, Artificial Sequence
  Synthetic
gaaagggggtggtaggggccgtacggtcatgccgtgcggttccgccacccctagggggccacacggtcctgccgtgtggttcccgctggttgtacagtgacgcattgggggccgtacggtcctgccgtgcggtttcctttgcttgctgtgcaatcggggatgacaccccctttcaacgtgggtactacgaaagtgcccctcgctccgaggttaaaggagaacccccccttcttacccccactcagctcgcccttcagtgcgggcgctagcctttccacttgcagcttctgcttgtagatgcttgcaccgtgattggtgcgcttcttgctttagtcgcttgtgcttctatcgttctgacgattcagtttcctaacgccagtgtttcgacggcccaagggggtagttgcggttagtattcctaccgcaattatccctttccccgttcgtagctggtttggatcttggatctctctccttccttcccccgtcttcaatttagcttcgtgattgaagcatctcactgtctctagtatttatgtcggactgacgattgagtacgttcagattgtgtttgggaggcccaagggatcgatggacaacacttcgaaagagtcacttgtccaccgctcctttcccctaccctagcaactctggatttgctcacgtggagttcgaggtctgtactttaactctgacttgcttttcttaccttgctatcttgctgacgtggattggttgtagactgattcacgttctcgttagatgctgacgtggagtacgatcgctgtacattccactactgccaattagctcccccttcccgttgctcccctctataaggagagccttctcttgcaaaggtgaagccttcacccccggtcgaagccgcttggaataagacagggttattttctcctctcctcggcgcttgcctcttctaagctgaataggttctatctattcaggcggatggtctggtccgttccttcttggacagagtgtgtatctgggttttccggatctcgaccacacactcaccagagctcaggagtgattaagtcaaggcccgatctgcggcgaaaaggaaatgaagtattttgcagctgtagcgacctctcaaggccagcggatttccccacctggtgacaggtgcctctggggccaaaagccacgtgttaatagcacccttgagagcggtggtaccccaccaccctgcaaattatggatttgacttagtaactaaaagattgacttggcatacctcaacctgagcggcggctaaggatgccctgaaggtacccntgntgaaatcgctnnggcgaccatggatctgatnaggggccctgcctggagtggntctatcccacacagcgtagggttaaaaaacgtcnaaccgccccacaangaccccggcagggatgccggtttnctntttaccaantctngacactatggcacaccatgacggaattccgtgtgagagctcttgccctcttgtcnacgccantgctgtcnacnacnagntcgntcttcttcanctccctgagcaggagccagaggtttatccgctggnnctgctcntttgtgatntggaagacgacgtnttcnacnctnctnccccggatcctgacccggaaccaatggattgttctgaattcgtacattcaaggccaaattctcctatggangttgacgacncagaagtcntggaaatctgctctatggagctcgatgagcagggcgctggatcatcaaanccatcaaccaacccaaatcagtcaggaaatacaggtacaattgtttataattactatgcaaatcagtaccaaaattcagtggatntntccggatccgcttcgagcgcttccggagcaccgactaagcccacaaatgcgcttggaagtgtgctttcagacgcaacctctgcctttgctactatggcgcctcttctcatggataatgacacagagacnatgaccaacttggctgacagngtttccacagacacgcaaggnaanacggccgtaaacactcaatcctcggtcggccgtctctgcgcttacggngcagagcacncaggngaancnccntcctcctgcgctgatgaaccnacatcagatgtccttgcagctcagaggtantacacnatnactggactnccngaatggacttcnacccaggantttcccagctttctgtanattcctctnccncangccctttccggtgaaannggnggtgttttcggngcnacnctccgnagncantacctntgnaaaacnggntggcgngttcaacttcagtgcaatgcttcncagttncantgtggntgnntnggnctnttnctngttccnganttnccncgcctnannnaccctttccngatttcnacnnnntgggangcnggctcggtntggggannngcncaaggtnannnnacnacctangccaacntctcnctngaccacatgaactactaccagatgtgnctntacccncatcaattnntgaatcttcgnacttcnacctcctgcagtgttgangtnccctncgtcaacatngctccntccagntcntggacccagcatgcnccctggagcatcntnatnatggtgctcncccctcttcnntactcagcnggntccactncctctctngatcttactgtntcnatngancctgtnaaacctgtcttnaatggcctncgtcanganacncttgttncncaggcnccnatcccagtnacaatcagagaacatcaaggttgcttctncactacnatgccngacaccaccgtgccnntcatgggnagaacaatnncntcnccncacgantacatgaaaggtgaggtcaaagancttgtntccattgcccanatncccaccttcctnggcaatgtnaanaacacnnacagantgccctacatctcnacntctgnnacncannnacnnctggcnaagtancaggtnacnctngcttgtgcttgcatgacnaacacttcncttggnnctcttgctngnaatttntctcantancgtggntctctntcntatgtntttgttttnactggttcngcnatggcnaanggtaagtttctnatntcntacacncccccnggtgcnggngancccancncagtngancangcnatgcagggaacctacgccatntggganntnggtctnaattcnacntggcantttacngtncccttcatntcncccacncactatcgnctcacntcctattcntctccntcnatnacntcngtnganggntggctnacngtttggcaactcacnggnatnacngtnccggctggngcncctccncantgtgangtnntnaccctnctnggtgctggagaagacttntcnntcaagatncccatncanncannnattccncttacngaacagggnnnngataatgcagagaanggtntngttnnagangagacngcngagtcngactttgtngcccacccnntntccncnccnggnaatcagacnntngtnganttcttctatgaccgnnctgtttgtgtnggnnnnntnnncgctancnntgcanntcngncccntgannnnngnccttctttcncanntnccntcncntaatggannncccnnncgctnnatnaanncncanncnggcaannnncgnntngnnggngttgcngatatnnnnncnntnttntatatgccttttacntattgtaaatatgatntngangtnacngcantngatntnnntnnnnanncggnnancnngnctttngtntncantatntnccnccaggtgcnccncnntanntnttntcnnnnnatcgngnnctnntnnccncannncanccccaagcngcnncnngnaatccctnnntnnttcagccntcagtngnnannngagnnntntnnctngnnntnttccctatgcntccccnctttcagttntnccngcngtttggtanaatggctatgnnactttnannaattcnggnnannnnggnnttgcnccngatgcnaatcttggtnnnnttgtnnctngntnnaannannnnngnaagannncttcagnttttcttncgntanaanaatttnagagnntggtgtccnngaccttcnnccttctncccctggccccannccacnnnnnnnaannntntnacannnganccnttnccagntcttgancttganatgccccgnntttctcgtgtctactgctttgngtttaantgncaggttggcntnctctangccaaactntttcagctttgccctcgttccagagccctctannntcagacntttgttacnganntcaanncattcacnngcttnaagcggtgggtgaanggntctccctanggaggnngatctcnttttacaaangaganntactccgccagagttctcttttttgaacgcccctanggctacaanatgcagtacaggtttggatgctcccnttcgaccaagaaagtntacaaggaactnncaatggaaaangtnatggcagagttnganttnttcagtcttcaaggntttganaattggcttcacncaccnntnnaagancaaggtgcngcaatttcncaccagtatgangaaatnccagacaggaaattcganncagctccaaatcnncccaaatgngatagacccanantggaaaagccnccnaagacnctctttaanttgcttaagaangttgtttcagaagatgaattggacccncttcagganctctggnncctnntcaanaanntngtnaaggcnttcaattcaatagttgatacacttcanaagccctanttntggattgcccaaattcggaaaatnaccaaattnatngcctacacagttctcatcaaacacaanccagatgcnaccacacttgcctgcgttgcagctcttgttgggacagaaatgctcgacaancgctccatcgtgganttnatnacaaagtgnttcanntcttggtttacaacnnctcccccngcnatgatggaagaacagatgcccaaaatgaaagacctnaatgantggttcactcttggnaagaacatagagtgggtcgtcaaaatgatnaaaaccctnttnaattggattacntcntggttcaagaangaagangagtctncncanggnaaactnaacaanctnctnctngactttgcngaaaatgcagananaatnaaaaantttagngcaggcaaaggcgttagacagtgcacccttaaggtgtctgtagcctanatgaaancagtctangatttggccatgaaagtaggaaanaccaanattgcctcngcagcttcaaaattcatggaagtnaanaancancacnnntcnagactngagcccgtngtcgtcgtnctncgcggcgcaccaggacaaggnaaatcagtnactgcccanatnttggctcaggcaatctccaaattggaaacaggaaancaatcagtgtattcagtnccaccagatgcaaattatntagatggntatgaaaancagcanacagtnatnatggatgatctaggncagaanccagatggaaaagantttgncaccttctgccanatggtgtcaaccaccaacttccttcccaanatggcttccctaganaataaaggaatncccttnacntccagagtcgtgctggccacgacaaatcancananattnaaccctgttaccatctctgangcnggcgccgttgatcgtcggatnaccttcgacntcaccgtccacgctcgctcagaatanagnaaaggcaggaccctagattttggaaaagcaatgcaacccatnccagatcanganccccctctcccntgcttnaanacacagtgccctctcctnaatggagangcngtttgcttcacaganaanaggactaatganaantacagcctngcagacattgtttgcntggtntgtgcagaactctcccaaaagaaaganacattggangtngcaaatgcnctngtnatgcantcaccagaaattgttatcactctagaacagatggaagaagcaatgaaaagtgtnttngaaacngcccaccaagtcaccacagaaganagagcagaactncttcangcnatnaangatgccctcaancatgcccaagtaatggatgattggatgaagatttcagctacctgtntgaatgtgatgcttgtggctttcaccggctaccagntctattcagcctggtcttcaaattctcaggaaaancccctcaaagttgtcattgatgcagcnaccgtcccaggtgaagaagaagcagcntacaatggaaaggtnaagaagaagaagacagagttgatcccaatgcagctagaagccccagcaatgtccccagattttgccaactatgttctnaagaaagtagtggcncccatgacccttcgctttganggcggaggtganttgacccagtcntgnntgatgattcgagancgaatnatcntttccaacaancatgccctctcnntagattggacncanatcaangtnaaaggactttggcacacncgtnnntccgtcaccattcaggcaatttgcaagggcggaaanacaacagacattgcagctgtgcgcctcccancaggcgancagtttaaggataatgttnnnaaattcatctcaaagaatgacccattcccantncccatgactcagatcaccggagtcaagaatgcaganacagcaacactttacacaggnacatttgtaaaggcccagacncagattttctcaacagcaggcaatcagtanggnaatgcnttncattananngcaaanacntttaaaggntattgtggctcagcaatttttggaaantgtggaaattcagacaaaataattggctttcactctgcaggcgcctcnggcgttgcagcaggnagcattctcacccgtgagatgctggaacaaatttgtgcaaatctaggaccaacccccctggaagaacaaggtgctctgaccctcattggcacaggngaagtntcncatgtcccaaggaagaccaanctcagncgctcattggcacacccncanttnaaacccaattatgatgtggcagttctntcaaaatangattcaagnactgacaaaaatgtagatgaagtttgntttcaaaaacatacnggcaacaanganaagctccaccccatcttngggctntanttnacagagtangctcanagagtnttcacacagctaggaacagataatngntgtctnaccatncaagaagcagttgatggngttgaaggaatggatgctatggaaanggatacctctccnggntngcccnnnnctctntcaggaaananaagagaanatgtttttgantttgaaaagaaacantttaaaagtgnannanncnnccncctcctanaggcanatggntnncnggagattantnctcntgtggnctaccaaagctttntgaaagangaaatncggnccntnnnaaaagtgcaagcancaaaaaccagantngntngatgtcccncccttcgagcattgcttgctcggaagacagtttctaggtaaatttgcagcaaagttttacaagaacccaggcacagtgcttggttcagcaattggctgtgatccagatacagattggactaaatttgcagttgccctaagccagtacaagtatgtttatgatgttgattactcaaattttgattctactcatggtacaggcatttttgaattggctatctccaaattcttcaatgttagaaatggatttgatccacgcacaggtaactacctgcgcagcctagcaacctcagtacacgcgtatgaggatgcaaggtaccagattgtaggtggactcccctcaggatgtgcagctactagtctcctcaatacagtgtttaataatgtcatcattagagcagggctagctcttacatataaaaattttgattacgatgacattgaagttttggcctacggcgacgacttgctcgttgcttcaaatttcaaaatagattttaatttggtcaaaaataacctctcaaaagaaggttacaaaattactcctgctagtaaaggtgatactttcccactagagagcactctggatgattgtgttttcttgaagagaaagtttgttaagaacgaccttgggctttacaaaccagtaatgtctgaggaagtcttgcaagctatgctttctttctacaaaccaggtaccctggcagagaagcttctgtccgtagccctacttgctgtccattctggacagaaagtttatgatcagtgctttgctccgtttcgcgaggctggcattgtgattccaggctatgacttggtgtatgatagatggcttagtcttcatcaatgaatggattggatttcggttgagcccccacccggtacaacgctttaccttagaagccactaaggtgtacgcggtcatcggggacccctcctggcctttggtttattggtgaattactagttcagttaggttttgttagttaggaaaaaaaaaaaaaaaaaaaaaaaaaa

SEQ ID NO: 98 - protein, 2305 aa, Artificial Sequence
  Synthetic
MAHHDGIPCESSCPLVXAXAVXXXXXLLXLPEQEPEVYPLXLLXCDLEDDVFXXXXPDPDPEPMDCSEFVHSRPNSPMEVDDXEVLEICSMELDEQGAGSSKPSTNPNQSGNTGTIVYNYYANQYQNSVDLSGSASSASGAPTKPTNALGSVLSDATSAFATMAPLLMDNDTETMTNLADRVSTDTQGNTAVNTQSSVGRLCAYGAEHXGEXPSSCADEPTSDVLAAQRYYTITGLPEWTSTQDFPSFLYIPLPHALSGEXGGVFGATLRRHYLCKTGWRVQLQCNASQFHCGCLGLFLVPEFPRLXXPFXISTXWXAGSVWGXAQGXXTTYANXSLDHMNYYQMCLYPHQFLNLRTSTSCSVEVPXVNIAPSSSWTQHAPWSIXIMVLXPLXYSAGSTXSLDLTVSIEPVKPVFNGLRHETLVXQAPIPVTIREHQGCFXTTMPDTTVPXMGRTIXSPHDYMKGEVKDLVSIAQIPTFLGNVKNTXRXPYISTSXTQXXLAKYQVTLACACMTNTSLGXLARNFSQYRGSLSYVFVFTGSAMAKGKFLISYTPPGAGEPXXVEQAMQGTYAIWDLGLNSTWQFTVPFISPTHYRLTSYSSPSITSVDGWLTVWQLTGITVPAGAPPQCDVLTLLGAGEDFSXKIPIQXXIPLTEQGXDNAEKGXVXDETAESDFVAHPXSXPGNQTLVDFFYDRXVCVGXXXAXXAXRPXXXXLLSHLPSXNGXPXRXIXXQXGNXRXXGVADIXXLFYMPFTYCKYDLEVTAXDXXXXXXXXFXLHYLPPGAPXYXFSXXRXLXXXXQPQAAXRNPXXXQPSXXTRXXSXVXPYASPLSVXPAVWYNGYXTFXNSGXXGXAPDANLGXXVXXXNXXGXXLQXFFRYKNFRXWCPRPSXFXPWPHXXXXKXXTXEPFPXLXLEMPRXSRVYCFXFKCQVGXLYAKLFQLCPRSRALYXQTFVTDXNXFTXFKRWVKGSPYGGXSXFTNEXYSARVLFFERPYGYKMQYRFGCSXSTKKVYKELXMENVMAEFDFFSLQGFXNWLHXPXXEQGAAISHQYEEIPDRKFDXAPNXPKCDRPXLEKPPKTLFNLLKKVVSEDELDPLQDLWXLXKKLVKAFNSIVDTLHKPYFWIAQIRKITKFIAYTVLIKHNPDATTLACVAALVGTEMLDNRSIVDFITKCFXSWFTTXPPAMMEEQMPKMKDLNDWFTLGKNIEWVVKMIKTLFNWITSWFKKEEESXQGKLNKLLLDFAENAEXIKNFRAGKGVRQCTLKVSVAYMKXVYDLAMKVGKTNIASAASKFMEVNNHHXSRLEPVVVVLRGAPGQGKSVTAQILAQAISKLETGKQSVYSVPPDANYLDGYENQHTVIMDDLGQNPDGKDFXTFCQMVSTTNFLPNMASLENKGIPFTSRVVLATTNHQXFNPVTISDAGAVDRRITFDXTVHARSEYRKGRTLDFGKAMQPIPDQEPPLPCFKTQCPLLNGEAVCFTDNRTNDNYSLADIVCLVCAELSQKKETLDVANALVMQSPEIVITLEQMEEAMKSVFETAHQVTTEERAELLQAIKDALNHAQVMDDWMKISATCLNVMLVAFTGYQXYSAWSSNSQEKPLKVVIDAATVPGEEEAAYNGKVKKKKTELIPMQLEAPAMSPDFANYVLKKVVAPMTLRFEGGGELTQSCLMIRXRIIXSNKHALSLDWTHIKVKGLWHTRXSVTIQAICKGGNTTDIAAVRLPXGDQFKDNVXKFISKNDPFPXPMTQITGVKNADTATLYTGTFVKAQTQIFSTAGNQYGNAFHYXANTFKGYCGSAIFGKCGNSDKIIGFHSAGASGVAAGSILTREMLEQICANLGPTPLEEQGALTLIGTGEVSHVPRKTKLRRSLAHPHFKPNYDVAVLSKYDSRTDKNVDEVCFQKHTGNKDKLHPIFGLYFTEYAQRVFTQLGTDNXCLTIQEAVDGVEGMDAMEXDTSPGXPXXLSGXXREXVFXFEKKQFKSXXAAASYRQMVAGDYSHVVYQSFLKDEIRPIEKVQAAKTRLVDVPPFEHCLLGRQFLGKFAAKFYKNPGTVLGSAIGCDPDTDWTKFAVALSQYKYVYDVDYSNFDSTHGTGIFELAISKFFNVRNGFDPRTGNYLRSLATSVHAYEDARYQIVGGLPSGCAATSLLNTVFNNVIIRAGLALTYKNFDYDDIEVLAYGDDLLVASNFKIDFNLVKNNLSKEGYKITPASKGDTFPLESTLDDCVFLKRKFVKNDLGLYKPVMSEEVLQAMLSFYKPGTLAEKLLSVALLAVHSGQKVYDQCFAPFREAGIVIPGYDLVYDRWLSLHQ

SEQ ID NO: 99 - protein, 7 aa, Artificial Sequence
  Synthetic
GXXGXKX

SEQ ID NO: 100 - protein, 5 aa, Artificial Sequence
  Synthetic
DDLXQ

SEQ ID NO: 101 - protein, 4 aa, Artificial Sequence
  Synthetic
GXCG

SEQ ID NO: 102 - protein, 5 aa, Artificial Sequence
  Synthetic
KDEXR

SEQ ID NO: 103 - protein, 6 aa, Artificial Sequence
  Synthetic
GGXPSG

SEQ ID NO: 104 - protein, 4 aa, Artificial Sequence
  Synthetic
YGDD

SEQ ID NO: 105 - protein, 4 aa, Artificial Sequence
  Synthetic
FLKR

SEQ ID NO: 106 - protein, 7 aa, Artificial Sequence
  Synthetic
GAPGQKS

SEQ ID NO: 107 - protein, 5 aa, Artificial Sequence
  Synthetic
DDLGQ

SEQ ID NO: 108 - DNA, 21 bp, Artificial Sequence
  Synthetic
gtgatgcttgtggctttcacc

SEQ ID NO: 109 - DNA, 22 bp, Artificial Sequence
  Synthetic
ctgcttcttcttcacctgggac

(19) United States
(12) Patent Application Publication
(10) Pub. No.: US 2014/0024015 A1
(43) Pub. Date: Jan. 23, 2014

(54) Boone Cardiovirus

(71) Applicant: IDEXX Laboratories, Inc., Westbrook, ME (US)

(72) Inventors: Lela Kay Riley, Columbia, MO (US); Judith D. Gohndrone, Orange, CA (US); Matthew Howard Myles, Moberly, MO (US)

(21) Appl. No.: 13/787,982

(22) Filed: Mar. 7, 2013

Related U.S. Application Data
(60) Provisional application No. 61/673,148, filed on Jul. 18, 2012.
(60) Provisional application No. 61/721,626, filed on Nov. 2, 2012.

(51) Int. Cl. C12Q 1/70

(52) U.S. Cl. CPC C12Q 1/70
USPC 435/5; 536/23.72; 530/300; 530/387.9; 435/320.1; 435/252.8; 435/348; 435/325; 435/358; 435/367

(57) ABSTRACT

The invention provides an isolated Boone cardiovirus, Boone cardiovirus polypeptides, polynucleotides and antibodies specific for Boone cardiovirus polypeptides. Also provided are methods for detection of Boone cardiovirus.

### PRIORITY

This application claims the benefit of U.S. Provisional application 61/673,148, filed Jul. 18, 2012, and U.S. Provisional application 61/721,626, filed Nov. 2, 2012, which are both incorporated herein by reference in their entirety.

### BACKGROUND OF THE INVENTION

Representing one of the oldest and more diverse viral families, picornaviruses are capable of causing disease in a wide range of hosts. Picornaviruses can cause asymptomatic infections or present with a wide range of clinical signs and symptoms including aseptic meningitis, encephalitis, the common cold, febrile rash, conjunctivitis, myocarditis, hepatitis, and diabetes. To date, the Picornaviridae family contains 12 recognized genera including; Aphthovirus, Avihepatovirus, Cardiovirus, Enterovirus, Erbovirus, Hepatovirus, Kobuvirus, Parechovirus, Sapelovirus, Seneca virus, Teschovirus, and Tremovirus (13). In addition, new viral strains that do not fit into defined genera are continually being discovered.

### SUMMARY OF THE INVENTION

In one embodiment, the invention provides an isolated polynucleotide molecule comprising: (a) SEQ ID NO:97; (b) a polynucleotide at least about 80%, 85%, 90%, 95%, 98% or more homologous to SEQ ID NO:97; (c) a polynucleotide comprising at least about 20 contiguous nucleic acids of SEQ ID NO:97, (d) GenBank accession number JQ864242 or JX683808, or (e) a complement of (a), (b), (c), or (d). The polynucleotide can be SEQ ID NO:5, 42-56, 69-83 or a polynucleotide comprising about 20 or more contiguous nucleic acids of SEQ ID NO:5, 42-56, 69-83 or a complement thereof.

Another embodiment of the invention comprises a substantially purified polypeptide encoded by a polynucleotide of the invention. A substantially purified polypeptide can have an amino acid sequence that is at least about 80%, 85%, 90%, 95%, or 98% identical to SEQ ID NO:98; an amino acid sequence that is at least about 80%, 85%, 90%, 95%, or 98% identical to a polypeptide comprising at least about 15 contiguous amino acids of SEQ ID NO:98; or an amino acid sequence of SEQ ID NO:35, 57-68, 84-96.

Yet another embodiment of the invention provides an isolated antibody or antigen binding fragment thereof that specifically binds to a substantially purified polypeptide of the invention.

Still another embodiment of the invention provides an isolated virus comprising a polynucleotide at least about 80%, 85%, 90%, 95%, or more identical to (a) SEQ ID NO:97, (b) GenBank accession number JQ864242, (c) GenBank accession number JX683808, or (d) a complement of (a), (b), or (c).

Even another embodiment of the invention provides an expression vector or host cell comprising an expression vector, wherein the expression vector comprises an isolated polynucleotide of the invention.

Still another embodiment of the invention provides a method of determining the presence or absence of Boone cardiovirus polynucleotides, polypeptides, or antibodies or specific binding fragments thereof that specifically bind to a Boone cardiovirus polypeptide comprising: (a) obtaining a test sample; and (b) determining the presence or absence of Boone Cardiovirus polynucleotides, polypeptides, or antibodies or specific binding fragments thereof in the test sample. The Boone cardiovirus can have a genome of GenBank accession number JQ864242 or JX683808, or a genome that is 85%, 90%, 95%, or 98% identical to GenBank accession number JQ864242 or JX683808, that is 85%, 90%, 95%, or 98% identical to SEQ ID NO:97, or a complement thereof. The test sample can be from a mammal that is subject to potential infection by Boone cardiovirus.

Another embodiment of the invention is a method of detecting a Boone cardiovirus polynucleotide. The method comprises amplifying polynucleotides of a sample (which can be suspected of containing a Boone cardiovirus polynucleotide) with at least one primer that hybridizes to at least 10 contiguous nucleic acids of SEQ ID NO:97, or a complement thereof, to produce an amplification product; and detecting the presence of the amplification product, thereby detecting the presence of the Boone cardiovirus polynucleotide. The method can comprise, for example, the use of at least two primers selected from (a) SEQ ID NO:108 and 109 or (b) SEQ ID NO:110 and SEQ ID NO:111. Optionally, one or more additional polynucleotides from one or more viruses, bacteria, fungi, or protozoans can also be detected. The polynucleotides can be amplified using a method selected from the group consisting of transcription mediated amplification (TMA), polymerase chain reaction (PCR), reverse-transcriptase PCR (RT-PCR), quantitative PCR, replicase mediated amplification, ligase chain reaction (LCR), competitive quantitative PCR (QPCR), real-time quantitative PCR, self-sustained sequence replication, strand displacement amplification, branched DNA signal amplification, nested PCR, in situ hybridization, multiplex PCR, Rolling Circle Amplification (RCA), and Q-beta-replicase system. The quantity of amplification products can be determined.

Yet another embodiment of the invention comprises a method of detecting the presence of Boone cardiovirus polynucleotides in a test sample. The method comprises contacting the sample with one or more isolated nucleic acid probes comprising about 10 or more contiguous nucleic acids of SEQ ID NO:97 and detecting the presence of hybridized probe/Boone cardiovirus nucleic acid complexes, wherein the presence of hybridized probe/Boone cardiovirus nucleic acid complexes indicates the presence of Boone cardiovirus in the test sample. The one or more probes can comprise one or more labels. Optionally, one or more additional polynucleotides from one or more viruses, bacteria, fungi, or protozoans can also be detected.

Still another embodiment of the invention comprises a method of detecting Boone cardiovirus polypeptides in a sample. The method comprises a) contacting the sample suspected of containing Boone cardiovirus polypeptides with an antibody of the invention (e.g., an antibody that specifically binds to a Boone cardiovirus polypeptide of the invention) to form Boone cardiovirus polypeptide/antibody complexes; and b) detecting the presence of the Boone cardiovirus polypeptide/antibody complexes, thereby detecting the presence of the Boone cardiovirus polypeptides. The polypeptide/antibody complexes can be detected by a technique comprising enzyme-linked immunosorbent assay (ELISA), multiplex fluorescent immunoassay (MFI or MFIA), radioimmunoassay (RIA), sandwich assay, Western blotting, immunoblotting analysis, an immunohistochemistry method, immunofluorescence assay, or a combination thereof. One or more additional polypeptides from one or more viruses, bacteria, fungi, or protozoans can also be detected.

Even another embodiment of the invention provides a method of detecting antibodies that specifically bind a Boone cardiovirus polypeptide in a test sample. The method comprises contacting one or more of the purified polypeptides of the invention with the test sample, under conditions that allow polypeptide/antibody complexes to form, and detecting the polypeptide/antibody complexes. The detection of the polypeptide/antibody complexes is an indication that antibodies specific for a Boone cardiovirus polypeptide are present in the test sample. The polypeptide/antibody complexes can be detected by a technique comprising enzyme-linked immunosorbent assay (ELISA), multiplex fluorescent immunoassay (MFI or MFIA), radioimmunoassay (RIA), sandwich assay, Western blotting, immunoblotting analysis, an immunohistochemistry method, immunofluorescence assay, or a combination thereof. Optionally, one or more additional antibodies that specifically bind one or more viruses, bacteria, fungi, or protozoans can also be detected.

Another embodiment of the invention provides a kit for detecting a Boone cardiovirus polynucleotides or polypeptides comprising at least one of: (a) SEQ ID NO:97; (b) one or more polynucleotides at least about 80%, 85%, 90%, 95%, 98% or more homologous to SEQ ID NO:97; (c) one or more polynucleotides comprising at least about 20 contiguous nucleic acids of SEQ ID NO:97; (d) one or more complements of (a), (b) or (c); (e) one or more substantially purified polypeptides encoded by the one or more polynucleotide of (a), (b), (c), or (d); (f) one or more isolated antibodies or antigen binding fragments thereof that specifically bind to the substantially purified polypeptides of (e); or (g) combinations thereof. The kit can further comprise one or more polynucleotides, one or more substantially purified polypeptides, one or more antibodies or antigen binding fragments that can detect one or more viruses, bacteria, fungi or protozoans other than Boone cardiovirus.

Therefore, the invention provides, inter alia, a novel virus, polynucleotides and polypeptides of the virus, antibodies specific for the polypeptides of the virus and methods and compositions for detecting the virus. The novel virus was isolated from the feces of asymptomatic laboratory rats. Both genetic and phylogenetic analyses demonstrate that this virus is a member of the Picornaviridae family and a novel species in the Cardiovirus genus. Characterizing novel rodent viruses and understanding the wide genetic diversity of viral families will aid the understanding of the clinical relevance of the ever growing list of “orphan” viruses for which no overt disease has been described. Additionally, methods of detection of Boon cardiovirus allows for identification of infected animals in research colonies. Infected animals can confound biological research by altering pathology, immune responses, and animal reproduction.

### BRIEF DESCRIPTION OF THE DRAWINGS

FIG. 1 shows the genome organization and conserved motifs of picornaviruses. The typical picornavirus genome consists of a 5′ untranslated region (UTR), a single open reading (polyprotein), a 3′ UTR, and a poly (A) tail. The polyprotein encodes three domains, P1, P2, and P3. The VP0 protein is a “proviral” protein that is cleaved into VP4 and VP2 during virus maturation in most picornavirus generas except Avihepatoviruses, Kobuviruses, and Parechovirues. The 2C protein contains two motifs conserved amongst picornaviruses GXXGXKX (X=any amino acid) (SEQ ID NO:99) and DDLXQ (SEQ ID NO:100). The 3C protein also has two conserved motifs GXCG (SEQ ID NO:101), the proteases active site, and GXH, involved in substrate binding. The 3D protein contains four conserved motifs involved in RNA template recognition and polymerase activity KDE[L/I]R (SEQ ID NO:102), GG[L/M/N]PSG (SEQ ID NO:103), YGDD (SEQ ID NO:104), and FLKR (SEQ ID NO:105).

FIG. 2 shows a phylogenetic tree based upon evolutionary relationships among the polyprotein sequences of picornaviruses. GenBank accession numbers are provided in the Examples section and for readability some strains used in alignments have been omitted from the figure. The tree was generated with MEGA5 using the neighbor-joining method. Branch confidence was assessed with bootstrap re-sampling of 1,000 pseudoreplicates. Evolutionary distances representing the number of amino acid differences per site were calculated using the p-distance method.

FIG. 3 shows pairwise amino acid identities matrixes comparing BCV with other members of the Cardiovirus genera. Three criteria for inclusion in an existing cardiovirus species were not met by BCV: sharing greater than 70% aa identity in the polyprotein (A), sharing greater than 60% aa identity in the P1 region (B), and sharing greater than 70% aa identity in the 2C+3CD region (C).

FIG. 4 shows alignment of cardiovirus and BCV Leader (L) proteins. Amino acid sequences were aligned as described in the Examples using ClustalW. There are four domains that make up the leader protein, the zinc finger motif, the acidic domain, the Ser/Thr domain, and the theilo domain. Amino acids that are involved in the EMCV phosphorylation site ([K/R]-X(2,3)-[E/D]-X(2,3)-Y) have been outlined. SEQ ID NO:8 is leader protein BCV; SEQ ID NO:9 is leader protein TRV; SEQ ID NO:10 is leader protein saffold-1; SEQ ID NO:11 is leader protein TMEV; SEQ ID NO:12 is leader protein Vilyuisk; SEQ ID NO:13 is leader protein EMCV.

FIG. 5A-B shows alignment of theilovirus and BCV Leader (L)* proteins. (A) The first lightly shaded AUG, indicates the initiation site of the polyprotein. The second darkly shaded AUG, represents the initiation site for the L* protein, which is located downstream and out of frame of the polyprotein initiation site. SEQ ID NO:14 is leader protein start codons for BCV; SEQ ID NO:15 is leader protein start codons for TRV; SEQ ID NO:16 is leader protein start codons for TMEV BEAN; SEQ ID NO:17 is leader protein start codons for TMEV WW; SEQ ID NO:18 is leader protein start codons for TMEV Yale; SEQ ID NO:19 is leader protein start codons for TMEV GDVII; SEQ ID NO:21 is leader protein start codons for TMEV FA; SEQ ID NO:22 is leader protein start codons for Saffold-1. FIG. 5B shows the amino acid alignment of predicted L* proteins of cardioviruses; BCV-1 (SEQ ID NO:35); TMEV DA (SEQ ID NO:36); TRV (SEQ ID NO:37); Vilyuisk (SEQ ID NO:38); TMEV GDVII (SEQ ID NO:39); SAFV-1 (SEQ ID NO:40); SAFV-2 (SEQ ID NO:41). The asterisk at the end of each sequence represents the stop codon.

FIG. 6 shows a similarity plot analysis based upon complete cardiovirus sequences using BCV as the query sequence. The y-axis shows the percent nucleotide similarity between the selected cardioviruses and the BCV query sequence. The x-axis indicates the nucleotide position within the genome and corresponds to the illustration at the top, which depicts the organization and relative size of the proteins within the genomes. The graph was generated using Simplot 3.5.1 as described in Example 4 with a sliding window of 300 bases and a step size of 10 bases.

FIG. 7 shows alignment of cardiovirus VP1 CD (A) and VP2 EF (B) loop sequences. (A) Regions that correspond with VP1 CD loops I and II have been shaded in gray. Amino acids that are identical to BCV have been outlined. (B) Regions that correspond to the VP2 EF loops I and II have been shaded in gray. Amino acids that are identical to BCV have been outlined. Amino acids of TMEV that have been implicated in binding sialic acid co-receptors on the surface of host cells have been indicated by the asterisks. SEQ ID NO:23 is VP1-CD loops for Vilyuisk; SEQ ID NO:24 is VP1-CD loops for TMEV; SEQ ID NO:25 is VP1-CD loops Saffold-1; SEQ ID NO:26 is VP1-CD loops for RTV; SEQ ID NO:27 is VP1-CD loops for EMCV; SEQ ID NO:28 is VP1-CD loops for BCV; SEQ ID NO:29 is VP2-EF loops for Vilyuisk; SEQ ID NO:30 is VP2-EF loops for TMEV; SEQ ID NO:31 is VP2-EF loops for Saffold-1; SEQ ID NO:32 is VP2-EF loops for RTV; SEQ ID NO:33 is VP2-EF loop for EMCV; SEQ ID NO:34 is VP2-EF loops for BCV.

### DETAILED DESCRIPTION OF THE INVENTION

As used herein, the singular forms “a,” “an,” and “the” include plural referents unless the context clearly dictates otherwise. The term “about” in association with a numerical value means that the numerical value can vary plus or minus by 5% or less of the numerical value.

Structurally, picornaviruses are non-enveloped positive-stranded RNA genomes that range from 7 to 9 kb in size. They encode a single open reading frame (ORF) that is translated into a polyprotein and subsequently cleaved by viral proteases to produce both structural and non-structural proteins. The polyprotein is preceded by a 5′ untranslated region (UTR), which plays an important role in viral replication, and followed by a 3′ UTR that regulates negative strand RNA synthesis.

A novel picornavirus that is related to members of the Cardiovirus genus has been identified. This genus includes two recognized species, Theilovirus and Encephalomyocarditis virus (EMCV), which share greater than 50% nucleotide identity. EMCV has been isolated from more than 30 host species including mammals, birds, and invertebrates. EMCV can cause a wide range of clinical manifestations including encephalitis, myocarditis, and diabetes. Theiloviruses can be divided into 12 genotypically different virus species; Theiler's murine encephalitis (TMEV), Thera virus (TRV), Saffold virus 1-9 (SAFV), and Vilyuisk human encephalomyelitis virus (VHEV). Strains of TMEV infect mice and can further be subdivided into two subgroups based upon the clinical presentation when mice are inoculated intracranially (18). The first group includes strains such as GDVII and FA. These strains are classified as highly neurovirulent strains of TMEV as they replicate in neurons and cause either acute or fatal polioencephalomyelitis. The second subgroup of TMEV is the Theiler's original strains (TO) including DA, BeAN, WW, and Yale strains. This second subgroup produces a biphasic infection. During the first phase, the virus replicates in the brain's gray matter causing subclinical encephalitis. During the second phase the virus migrates to the spinal cord and persists in the brain's white matter causing demyelination. During the persistent stage of infection the virus is found to replicate in macrophages (8, 27). TRV has only been isolated from asymptomatic rats and has yet to be associated with clinical disease (5, 21). The last two species within the Theilovirus genus, SAFV and VHEV have both been isolated from humans. There are 9 recognized serotypes of SAFV and these viruses are typically isolated from children. The clinical significance of SAFV is still being investigated; however, to date SAFV has been isolated from febrile infants, children with gastroenteritis, children with respiratory disease, and children who have died from SIDS (1, 2, 12, 19). VHEV is a geographically isolated virus that has only been found in individuals living in a specific region of Russia with a high prevalence of encephalomyelitis. It was first isolated from the cerebrospinal fluid of an adult with a neurodegenerative disease (9).

A novel picornavirus, Boone Cardiovirus (BCV), which was isolated from the feces of asymptomatic laboratory rats, is presented herein. Two strains of BCV have been identified: BCV-1 and BCV-2. Phylogenetic analysis shows BCV is a new species of cardiovirus that is equally divergent from both EMCV and Theilovirus species. The ICTV definitions for cardiovirus species determination state that a member of a species must share greater than 70% amino acid (aa) identity in the polyprotein, greater than 60% aa identity in the P1 region, greater than 70% aa identity in the 2C+3CD region, share a natural host range, and a common genome organization. BCV, when compared to either EMCV or Theiloviruses, satisfies only two of the five requirements and as a result should be considered a novel species within the cardiovirus genus.

Of 140 samples tested from 56 different facilities, 20% of rats were positive for BCV using RT-PCR. Previously, Thera virus (RTV) appeared to be the most prevalent rat virus with a prevalence of 2.0-2.5%. 30% of the 56 facilities tested had at least one positive animal. While BCV can be found in most organs (brain, heart, lung, liver, pancreas, spleen, kidney, duodenum, ileum, cecum, colon, epididymis, testis, prostate, seminal fluid, ovaries, uterus), it is consistently detected in the gastrointestinal tract with the highest titers observed in the duodenum. BCV infection is persistent and shedding in feces begins at about 5-6 weeks of age.

Phylogenetic analysis determined that BCV encodes an L protein that shares only some of the typically characteristics of other cardioviruses. Leader proteins have been identified in several picornaviruses such as Cardioviruses, Aphthoviruses, Erboviruses, Kobuviruses, Teschoviruses, and Sapeloviruses. The function of leader proteins has only been studied in the aphtho-, erbo-, and cardio-viruses. Leader proteins of aphtho- and erbo-viruses act as a papain-like cysteine proteinases that cleave eukaryotic initiation factors, resulting in the shut off of host protein synthesis (33, 34). In cardioviruses, the L protein is believed to play a critical role in cytosol-dependent phosphorylation cascades involved in nucleocytoplasmic trafficking and cytokine expression (6, 7, 24).

There are four defined properties of cardiovirus leader proteins including a zinc finger motif, an acidic domain, a serine/threonine-rich (ser/thr) domain, and a theilo domain. The zinc finger and acidic domains are conserved amongst all cardioviruses; whereas the ser/thr and theilo domains are present in only some theilovirus subspecies. Only the acidic and the ser/thr domains were identified in BCV. The ser/thr domain is found in TMEV, VHEV, and TRV, but is partially deleted in SAFV. The most unique feature of the BCV L protein as compared to other cardioviruses is the lack of an identifiable zinc finger, which has been identified in all other species. Historically, when the zinc finger motif was removed from TMEV in vitro, apoptosis of infected cells was not observed (3, 7). Apoptosis is a method of viral spread during infection and this deficiency can attenuate viral infections. Dvorak et al. observed that deletion of the zinc finger motif in EMCV led to restricted infections and reduced protein synthesis (6). To date, BCV has not been propagated in cell culture despite attempts in over fifteen different cell lines and varied growth conditions, whether the lack of a zinc finger motif in the L protein can contribute to these difficulties has yet to be determined. In vivo, zinc finger mutations reduced viral titers of persistent TMEV in the spinal cords of mice (25). Mutations in the zinc finger motif have also been shown to decrease the anti-alpha/beta interferon responses during viral infections (3, 4, 7, 29).

Despite the evidence that zinc fingers in the leader protein play an important role in cardiovirus infections, evidence suggests that the domains of the L protein act synergistically. Ricour et al. generated independent mutations in the zinc finger and theilodomains and showed that these mutations affected all of the L protein functions that were tested including nucleocytoplasmic trafficking and interferon responses (24). This is further supported by the fact that the EMCV L protein does not encode the theilo or ser/thr domains; however, it has retained the ability to modulate the same processes as theiloviruses (22). More recently discovered picornaviruses, such as Mouse kobuvirus and Senecavirus also encode cardiovirus-like L proteins, but lack the zinc finger motif similar to BCV (10, 23).

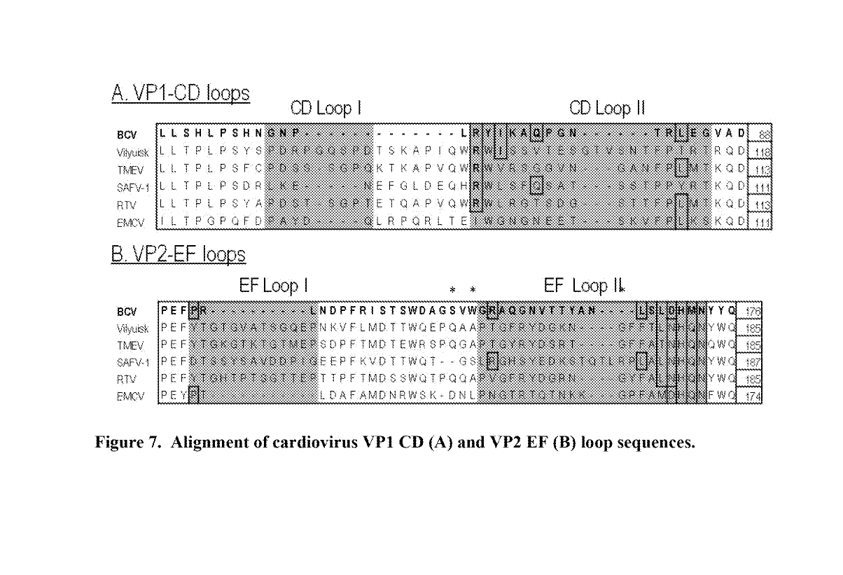

Laboratory rats can be persistently infected with BCV. By RT-PCR, continual fecal shedding can be detected from naturally infected rats 5 weeks to 10 months of age. In TO strains of TMEV the L* protein plays a crucial role in viral growth in macrophages and persistence infections of the host (26, 27). Analysis of the BCV genome predicts that like the TO TMEV strains it produces a functional L* protein. A second characteristic of TO TMEV strains that has been shown to be associated with persistence is the use of sialic acid as a co-receptor for viral entry. Three amino acids (FIG. 7b) of the VP2 protein have been identified as playing a direct role in the binding of sialic acid (16, 30). These amino acids are conserved in non-persistent TMEV strains; however, it has been suggested that the overall protein structure inhibits sialic acid binding. These amino acids are not conserved by BCV. In the case of BCV, it is more likely that persistence is encoded by the L* protein or by another unidentified genomic element than, by the binding of sialic acid.

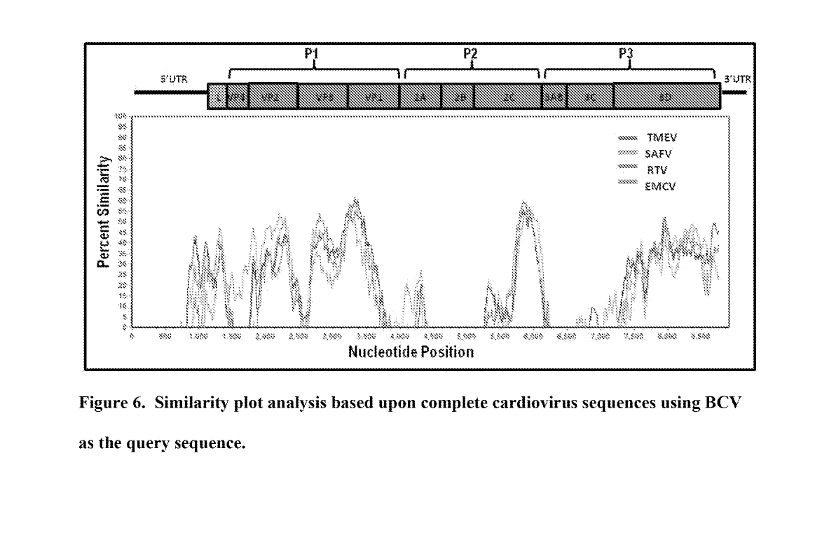

Cardioviruses have exposed surfaces on their capsids that are involved in host cell tropism and act as immunogenic sites that can affect virulence. These sites are the CD and EF loops located within the VP1 and VP2 proteins respectively. Despite the fact, some regions of highest shared amino acid identity between BCV-1 and cardioviruses are found in these capsid regions (FIG. 6), BCV-1 shares very little amino acid identity in either of the CD and EF loops (FIG. 7). This indicates that the exposed surface of BCV mostly likely has a unique secondary structure as compared to known cardioviruses and suggests that BCV has the potential to enter cells through a different host receptor.

BCV's failure to propagate efficiently in cell culture has hindered the ability to purify and concentrate virus for controlled in vivo studies to determine its biological significance; however, BCV is a seemingly non-pathogenic virus as infected rats do not present with clinical symptoms. Despite appearing non-pathogenic due to the persistent nature of BCV infections the long term consequences and subclinical impact of infection on the host needs to be evaluated in future studies. Understanding BCV infection may be useful in further understanding the difference between aspects of the cardiovirus genome that contribute to clinical symptoms in both rodents and humans and the regions that do not. Most likely BCV does not go undetected by the host immune system and understanding how the virus is kept in check may hold clues to identifying novel antivirals for the pathogenic strains of cardioviruses and other picornaviruses. BCV may also prove useful as a comparative strain for understanding the many “orphan” viruses that have cardiovirus-like elements, such as Mouse kobuvirus, Senecaviruses, and Mosavirus; that have recently been discovered, but for which relatively nothing is known and no overt disease has been identified. Picornaviruses such as BCV can also be useful to establish models of human disease. For example, TMEV is a model for multiple sclerosis and coxsackie B virus is a model for diabetes mellitus.

Additionally, the detection of BCV in laboratory animals including mice and rats is important because viruses may confound biological research by altering pathology, altering the immune system or altering animal reproduction.

### Polynucleotides

Polynucleotides of the invention comprise isolated nucleic acid molecules comprising SEQ ID NOs:5, 42-56, 69-83, 97, fragments thereof, complements thereof, reverse sequences thereof, or combinations thereof. An isolated polynucleotide of the invention hybridizes under stringent conditions to at least 20, 30, 40, 50, 60, 70, 80, 90, 100 or more contiguous nucleotides of a nucleotide sequence set forth in SEQ ID NO:5, 42-56, 69-83, 97, or a complement thereof. The stringent conditions can comprise, for example, hybridizing at 37° C. in a buffer of 40% formamide, 1 M NaCl, 1% SDS and washing in 1×SSC at 45° C. An isolated polynucleotide of the invention includes a nucleic acid sequence that is at least about 80%, 85%, 90%, 91%, 92%, 93%, 94%, 95%, 96%, 97%, 98%, 99%, 99.5% or more homologous to (a) SEQ ID NO:5, 42-56, 69-83, 97 (b) a polynucleotide comprising at least about 20, 30, 40, 50, 60, 70, 80, 90, 100 or more contiguous nucleic acids of SEQ ID NO:5, 42-56, 69-83, 97 or (c) a complement thereof. Other isolated polynucleotides of the invention include, for example, SEQ ID NO:5, 42-56, 69-83, 97 or a polynucleotide comprising 20, 30, 40, 50, 60, 70, 80, 90, 100 or more contiguous nucleic acids of SEQ ID NO:5, 42-56, 69-83, 97.

A complement is a nucleic acid molecule that, when aligned anti-parallel to a target nucleic acid molecule, has complementary nucleic acid bases to the target molecule at each nucleotide position.

One embodiment of the invention provides one or more of the following polynucleotides: SEQ ID NO:5 (whole BCV-1 genome), SEQ ID NO:42 (BCV-1 5′ UTR polynucleotide), SEQ ID NO:43 (BCV-1 Leader nucleic acid sequence), SEQ ID NO:44 (BCV-1 Leader* nucleic acid sequence), SEQ ID NO:45 (BCV-1 VP4 nucleic acid sequence), SEQ ID NO:46 (BCV-1 VP2 nucleic acid sequence), SEQ ID NO:47 (BCV-1 VP3 nucleic acid sequence), SEQ ID NO:48 (BCV-1 VP1 nucleic acid sequence), SEQ ID NO:49 (BCV-1 2A nucleic acid sequence), SEQ ID NO:50 (BCV-1 2B nucleic acid sequence), SEQ ID NO:51 (BCV-1 2C nucleic acid sequence), SEQ ID NO:52 (BCV-1 3A nucleic acid sequence), SEQ ID NO:53 (BCV-1 3B nucleic acid sequence), SEQ ID NO:54 (BCV-1 3C nucleic acid sequence), SEQ ID NO:55 (BCV-1 3D nucleic acid sequence), SEQ ID NO:56 (BCV-1 3′ UTR nucleic acid sequence), SEQ ID NO:69 (partial BCV-2 genome), SEQ ID NO:70 (BCV-2 nucleotide sequence of polyprotein), SEQ ID NO:71 (BCV-2 Leader nucleic acid sequence), SEQ ID NO:72 (BCV-2 Leader* nucleic acid sequence), SEQ ID NO:73 (BCV-2 VP4 nucleic acid sequence), SEQ ID NO:74 (BCV-2 VP2 nucleic acid sequence), SEQ ID NO:75 (BCV-2 VP3 nucleic acid sequence), SEQ ID NO:76 (BCV-2 VP1 nucleic acid sequence), SEQ ID NO:77 (BCV-2 2A nucleic acid sequence), SEQ ID NO:78 (BCV-2 2B nucleic acid sequence), SEQ ID NO:79 (BCV-2 2C nucleic acid sequence), SEQ ID NO:80 (BCV-2 3A nucleic acid sequence), SEQ ID NO:81 (BCV-2 3B nucleic acid sequence), SEQ ID NO: 82 (BCV-2 3C nucleic acid sequence), SEQ ID NO:83 (BCV-2 3D partial nucleic acid sequence), SEQ ID NO:97 (consensus sequence of SEQ ID NO:5 and SEQ ID NO:69).

Polynucleotides of the invention can be naturally occurring nucleic acid molecules, recombinant nucleic acid molecules, or synthetic polynucleotides. A polynucleotide also includes amplified products of itself, for example, as in a polymerase chain reaction. A polynucleotide can be a fragment of a Boone cardiovirus nucleic acid molecule or a whole Boone cardiovirus nucleic acid molecule. Polynucleotides of the invention can be about 10, 20, 30, 40, 50, 100, 200, 300, 400, 500, 600, 700, 800, 900, 1,000, 1,200, 1,300, 1,500, 2,000, 3,000, 4,000, 5,000, 6,000, 7,000, 8,000 or more nucleic acids in length. A polynucleotide fragment of the invention can comprise about 10, 20, 30, 40, 50, 100, 200, 300, 400, 500, 600, 700, 800, 900, 1,000, 1,200, 1,300, 1,500, 2,000, 3,000, 4,000, 5,000, 6,000, 7,000, 8,000 or more contiguous nucleic acids (or any range or value between about 10 and 6,000 contiguous nucleic acids) of SEQ ID NO:5, 42-56, 69-83, 97. A polynucleotide fragment of the invention can comprise about 8,000, 7,000, 6,000, 5,000, 4,000, 3,000, 2,000, 1,500, 1,300, 1,200, 1,000, 900, 800, 700, 600, 500, 400, 300, 200, 100, 50, 40, 30, 20, 10 or less contiguous nucleic acids (or any range or value between about 6,000 and 10 contiguous nucleic acids) of SEQ ID NO:5, 42-56, 69-83, or 97. A polynucleotide fragment of the invention can be, for example, about 10-50, 25-75, 50-100, 50-200, 50-300, 100-300, 250-500, 300-600, 400-600, 500-750, 500-1,000, 750-1250 nucleotides in length.

A nucleic acid, nucleic acid molecule, polynucleotide, or polynucleotide molecule refers to covalently linked sequences of nucleotides (i.e., ribonucleotides for RNA and deoxyribonucleotides for DNA) in which the 3′ position of the pentose of one nucleotide is joined by a phosphodiester group to the 5′ position of the pentose of the next. A polynucleotide can be RNA, DNA, cDNA, genomic DNA, chemically synthesized RNA or DNA, or combinations thereof. A nucleic acid molecule can comprise chemically, enzymatically or metabolically modified forms of nucleic acids.

SEQ ID NO:97 comprises a consensus polynucleotide of SEQ ID NO:5 (BCV-1) and SEQ ID NO:69 (BCV-2). The alignment of BCV-1 SEQ ID NO:5 and BCV-2 SEQ ID NO:69 is shown below in the Sequences section. In the consensus sequence (SEQ ID NO:97) an X represents any nucleotide or an absent nucleotide. In one embodiment of the invention, the X represents either of the two nucleotides (or absent nucleotide) that occur at that position in the alignment of BCV-1 SEQ ID NO:5 and BCV-2 SEQ ID NO:69, which is shown below in the Sequences section. For example, in the alignment the nucleotide at position 1441 of BCV-1 is T and the nucleotide at position 187 of BCV-2 (which aligns with position 1441 of BCV-1) is A. Therefore, in the consensus sequence the X for this position can be A or T. This is also true for each smaller polynucleotide and fragment sequence. For example, polynucleotide VP4 (SEQ ID NO: 45 for BCV-1 and SEQ ID NO:73 for BCV-2) have several X's within the consensus sequence. The X in the consensus sequence (SEQ ID NO:97) at 1757 can be any nucleotide. In another embodiment the X in the consensus sequence (SEQ ID NO:97) at 1757 can be G, which is the corresponding nucleotide in BCV-1 VP4 (nucleotide 12 of SEQ ID NO:45) or it can be A, which is the corresponding nucleotide in BCV-2 VP4 (nucleotide 12 of SEQ ID NO:73). Other examples in VP4 include: the X at 1842 of consensus sequence SEQ ID NO:97 can be T (position 97 of BCV-1 SEQ ID NO:45) or C (position 97 of BCV-2 SEQ ID NO:73) and the X at position 1844 of consensus sequence SEQ ID NO:97 can be G (position 99 of BCV-1 SEQ ID NO:45) or C (position 99 of BCV-2 SEQ ID NO:73). The same analysis can be used to determine 2 alternate nucleotides for each X in consensus sequence SEQ ID NO:97 for each genome, 5′UTR, leader, leader*, VP4, VP2, VP3, VP1, 2A, 2B, 2C, 3A, 3B, 3C, and 3D polynucleotide.

Nucleic acid molecules of the invention can also include, for example, polydeoxyribonucleotides (containing 2-deoxy-D-ribose), polyribonucleotides (containing D-ribose), any other type of polynucleotide which is an N- or C-glycoside of a purine or pyrimidine base, and other polymers containing normucleotidic backbones, for example, polyamide (e.g., peptide nucleic acids (PNAs)) and polymorpholino polymers, and other synthetic sequence-specific nucleic acid polymers providing that the polymers contain nucleobases in a configuration which allows for base pairing and base stacking, such as is found in DNA and RNA. Nucleic acid molecules also include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates), with negatively charged linkages (e.g., phosphorothioates, phosphorodithioates), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. A nucleotide analog refers to a nucleotide in which the pentose sugar and/or one or more of the phosphate esters is replaced with its respective analog.

The polynucleotides can be purified free of other components, such as proteins, lipids and other polynucleotides. For example, the polynucleotide can be 50%, 75%, 90%, 95%, 96%, 97%, 98%, 99%, or 100% purified. Polynucleotides of the invention can comprise other nucleotide sequences, such as sequences coding for labels, linkers, signal sequences, TMR stop transfer sequences, transmembrane domains, or ligands useful in protein purification such as glutathione-S-transferase, histidine tag, and staphylococcal protein A.

Polynucleotides of the invention can contain less than an entire viral genome or the entire viral genome. Polynucleotides of the invention can be isolated. An isolated polynucleotide that is less than the entire viral genome is a polynucleotide that is not immediately contiguous with one or both of the 5′ and 3′ flanking genomic sequences that it is naturally associated with. An isolated polynucleotide that is less than the entire viral genome can be, for example, a recombinant DNA or RNA molecule of any length, provided that the nucleic acid sequences naturally found immediately flanking the recombinant DNA or RNA molecule in a naturally-occurring genome is removed or absent. An isolated polynucleotide that comprises the entire viral genome is substantially isolated away from other polynucleotides, capsid proteins, proteases, and biological or environmental sample remnants. Isolated polynucleotides can be naturally-occurring or non-naturally occurring nucleic acid molecules. A nucleic acid molecule existing among hundreds to millions of other nucleic acid molecules within, for example, cDNA or genomic libraries, or gel slices containing a genomic DNA restriction digest are not to be considered an isolated polynucleotide.

Polynucleotides of the invention can comprise naturally occurring BCV sequences or can comprise altered sequences that do not occur in nature. If desired, polynucleotides can be cloned into an expression vector comprising expression control elements, including for example, origins of replication, promoters, enhancers, or other regulatory elements that drive expression of the polynucleotides of the invention in host cells. An expression vector can be, for example, a plasmid, such as pBR322, pUC, or ColE1, a baculovirus vector, or an adenovirus vector, such as an adenovirus Type 2 vector or Type 5 vector. Optionally, other viral vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Mini-chromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

Methods for preparing polynucleotides operably linked to an expression control sequence and expressing them in a host cell are well-known in the art. See, e.g., U.S. Pat. No. 4,366,246. A polynucleotide of the invention is operably linked when it is positioned adjacent to or close to one or more expression control elements, which direct transcription and/or translation of the polynucleotide.

Polynucleotides of the invention can be isolated from nucleic acid molecules present in, for example, a biological sample, such as blood, serum, feces, cells, saliva, or tissue from an infected individual. Polynucleotides can also be synthesized in the laboratory, for example, using an automatic synthesizer. An amplification method such as PCR can be used to amplify polynucleotides from genomic RNA, DNA or cDNA encoding polypeptides of the invention.

Polynucleotides of the invention can be used, for example, as probes or primers, for example PCR primers, to detect BCV polynucleotides in a sample, such as a biological sample or an environmental sample. The ability of such probes and primers to specifically hybridize to BCV polynucleotide molecules will enable them to be of use in detecting the presence, absence and/or quantity of complementary nucleic acid molecules in a given sample. Polynucleotide probes and primers of the invention can hybridize to complementary sequences in a sample such as a biological sample or environmental sample. Polynucleotides from the sample can be, for example, subjected to gel electrophoresis or other size separation techniques or can be immobilized without size separation. The polynucleotides from the sample are contacted with the probes or primers under hybridization conditions of suitable stringencies.

A probe is a nucleic acid molecule of the invention comprising a sequence that has complementarity to a BCV nucleic acid molecule of the invention and that can hybridize to the BCV nucleic acid molecule.

A primer is a nucleic acid molecule of the invention that can hybridize to a BCV nucleic acid molecule through base pairing so as to initiate an elongation (extension) reaction to incorporate a nucleotide into the nucleic acid primer. The elongation reactions can occur in the presence of nucleotides and a polymerization-inducing agent such as a DNA or RNA polymerase and at suitable temperature, pH, metal concentration, and salt concentration.

Polynucleotide hybridization involves providing denatured polynucleotides (e.g., a probe or primer or combination thereof and BCV nucleic acid molecules) under conditions where the two complementary (or partially complementary) polynucleotides form stable hybrid duplexes through complementary base pairing. The polynucleotides that do not form hybrid duplexes can be washed away leaving the hybridized polynucleotides to be detected, e.g., through detection of a detectable label. Alternatively, the hybridization can be performed in a homogenous reaction where all reagents are present at the same time and no washing is involved.

In one embodiment, a polynucleotide molecule of the invention comprises one or more labels. A label is a molecule capable of generating a detectable signal, either by itself or through the interaction with another label. A label can be a directly detectable label or can be part of a signal generating system, and thus can generate a detectable signal in context with other parts of the signal generating system, e.g., a biotin-avidin signal generation system, or a donor-acceptor pair for fluorescent resonance energy transfer (FRET). The label can, for example, be isotopic or non-isotopic, a catalyst, such as an enzyme, a polynucleotide coding for a catalyst, promoter, dye, fluorescent molecule, chemiluminescer, coenzyme, enzyme substrate, radioactive group, a small organic molecule, amplifiable polynucleotide sequence, a particle such as latex or carbon particle, metal sol, crystallite, liposome, cell, a colorimetric label, catalyst or other detectable group. A label can be a member of a pair of interactive labels. The members of a pair of interactive labels interact and generate a detectable signal when brought in close proximity. The signals can be detectable by visual examination methods well known in the art, preferably by FRET assay. The members of a pair of interactive labels can be, for example, a donor and an acceptor, or a receptor and a quencher.

### Hybridization Conditions

Hybridization and the strength of hybridization (i.e., the strength of the association between polynucleotide strands) is impacted by many factors well known in the art including the degree of complementarity between the polynucleotides, length, stringency of the hybridization conditions, e.g., conditions as the concentration of salts, the thermal melting temperature (Tm) of the formed hybrid, the presence of other components (e.g., the presence or absence of polyethylene glycol), the molarity of the hybridizing strands and the G:C content of the polynucleotide strands. Tm is the temperature (at defined ionic strength, pH, and nucleic acid concentration) at which 50% of a polynucleotide molecule and its perfect complement are in a double-stranded duplex.

Under high stringency conditions, polynucleotide pairing will occur only between polynucleotide molecules that have a high frequency of complementary base sequences. Generally, high stringency conditions can include a temperature of about 5 to 20° C. lower (e.g., about 5, 10, 15, 20° C. or lower) than the Tm of a specific nucleic acid molecule at a defined ionic strength and pH. An example of high stringency conditions comprises a washing procedure including the incubation of two or more hybridized polynucleotides in an aqueous solution containing 0.1×SSC and 0.2% SDS, at room temperature for 2-60 minutes, followed by incubation in a solution containing 0.1×SSC at room temperature for 2-60 minutes. Another example of high stringency conditions comprises hybridizing at 42° C. in a solution comprising 50% formamide, 5×SSC, and 1% SDS and washing at 65° C. in a solution comprising 0.2×SSC and 0.1% SDS. An example of stringent conditions is hybridization at 37° C. in a buffer of 40% formamide, 1 M NaCl, 1% SDS and a wash in 1×SSC at 45° C. An example of low stringency conditions comprises a Tm of about 25-30° C. below Tm and a washing procedure including the incubation of two or more hybridized polynucleotides in an aqueous solution comprising 1×SSC and 0.2% SDS at room temperature for 2-60 minutes. Stringency conditions are known to those of skill in the art, and can be found in, for example, Sambrook et al. (1989) Molecular Cloning: A Laboratory Manual (2nd ed., Cold Spring Harbor Laboratory); Berger and Kimmel, eds., (1987) “Guide to Molecular Cloning Techniques”, In Methods in Enzymology: 152: 467-469; and Anderson and Young (1985) “Quantitative Filter Hybridisation.” In: Hames and Higgins, eds., Nucleic Acid Hybridisation, A Practical Approach. Oxford, IRL Press, 73-111.

Stringency conditions can be adjusted to screen for moderately similar fragments such as homologous sequences from related organisms, or to highly similar fragments. The stringency can be adjusted either during the hybridization step or in the post-hybridization washes. Salt concentration, formamide concentration, hybridization temperature and probe lengths are variables that can be used to alter stringency.

Polynucleotide sequences that hybridize to the claimed polynucleotide sequences, including any of the nucleic acid sequences disclosed herein, and fragments thereof under stringent and/or highly stringent conditions are included in the invention. See, e.g., Wahl and Berger (1987) Methods Enzymol. 152: 399-407; Kimmel (1987) Methods Enzymol. 152: 507-511.

In general, stringency is determined by the temperature, ionic strength, and concentration of denaturing agents (e.g., formamide) used in hybridization and washing procedures. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having about 80%, 85%, 90%, 95%, 96%, 97%, 98%, 99%, 99.5% or greater identity with disclosed nucleic acid sequences.

Hybridization experiments are generally conducted in a buffer of pH between 6.8 to 7.4, although the rate of hybridization is nearly independent of pH at ionic strengths likely to be used in the hybridization buffer. In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringent salt concentration will ordinarily be less than about 750 mM NaCl and 75 mM trisodium citrate. High stringency conditions can be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide.

The wash steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

### Sequence Identity

Percent sequence identity has an art recognized meaning and there are a number of methods to measure identity between two polypeptide or polynucleotide sequences. Sequence identities can be determined by analysis with a sequence comparison algorithm or by a visual inspection. Polypeptide and polynucleotide molecule identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window is a segment of any one of the number of contiguous positions selected from the group consisting of from about 20 to about 600 (for example from about 50-200, 100-150, 10-50, 100-150, 50-200) in which a sequence can be compared to a reference sequence of the same number of contiguous positions after the two sequences are optimally aligned. Methods of alignment of sequences for comparison are well-known in the art. In one embodiment of the invention only about 10-20, 10-50, 10-100, 10-200, 10-250, 10-300, 10-400, 10-500, 10-600, 10-700, 10-800 amino acids or nucleotides are compared.

An algorithm suitable for determining percent sequence identity and sequence similarity includes, e.g., the FASTA algorithm (Pearson & Lipman, Proc. Natl. Acad. Sci. U.S.A. 85: 2444, 1988; Pearson, Methods Enzymol. 266: 227-258, 1996). Exemplary parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16. BLAST and BLAST 2.0 algorithms can also be used to determine percent sequence identity and sequence similarity (Altschul et al., Nuc. Acids Res. 25:3389-3402, 1977; Altschul et al., J. Mol. Biol. 215:403-410, 1990). The BLASTN program (for nucleotide sequences) uses as defaults a wordlength (W) of 11, an expectation (E) of 10 μM=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10 μM=5, N=−4, and a comparison of both strands.

Another algorithm that can be used is PILEUP. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0. See, Devereaux et al., Nuc. Acids Res. 12:387-395, 1984.

Another example of an algorithm that can be used for multiple DNA and amino acid sequence alignments is the CLUSTALW program (Thompson et al., Nucl. Acids. Res. 22:4673-4680, 1994). ClustalW performs multiple pairwise comparisons between groups of sequences and assembles them into a multiple alignment based on homology. Gap open and Gap extension penalties can be 10 and 0.05 respectively. For amino acid alignments, the BLOSUM algorithm can be used as a protein weight matrix. See, Henikoff & Henikoff, Proc. Natl. Acad. Sci. U.S.A. 89:10915-10919, 1992.

Substantially homologous nucleotide sequences and complements thereof are also polynucleotides of the invention. Homology refers to the percent similarity between two polynucleotides. Two polynucleotide sequences are “substantially homologous” to each other when the sequences exhibit at least about 80%, 85%, 90%, 91%, 92%, 93%, 94%, 95%, 96%, 97%, 98%, 99%, 99.5% or 100% sequence similarity over a defined length of the molecules. As used herein, substantially homologous also refers to sequences showing complete identity to the specified polynucleotide sequence.

When using any of the sequence alignment programs to determine whether a particular sequence is, for instance, about 95% identical to a reference sequence, the parameters can be set such that the percentage of identity is calculated over the full length of the reference polynucleotide and that gaps in identity of up to 5% of the total number of nucleotides in the reference polynucleotide are allowed.

Percent identity in the context of two or more nucleic acids or polypeptide sequences, refers to the percentage of nucleotides or amino acids that two or more sequences or subsequences contain which are the same over a specified length, e.g., 10, 15, 20, 30, 40, 50, 60, 70, 80, 90, 100, 200, 300, 400, 500, 600, 700, 800, 900, 1,000, 1,250, 1,500, 1,750, 2,000, 2,500, 3,000 or more nucleotides or amino acids. A specified percentage of amino acid residues or nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

Substantially identical in the context of two polynucleotides or polypeptides, refers to two or more sequences or subsequences that have at least of at least about 80%, 85%, 90%, 91%, 92%, 93%, 94%, 95%, 96%, 97%, 98%, 99%, 99.5% or higher nucleotide or amino acid residue identity, when compared and aligned for maximum correspondence, as measured using one of the following sequence comparison algorithms or by visual inspection.

### Polypeptides

A polypeptide is a polymer of two or more amino acids covalently linked by amide bonds. A polypeptide can be post-translationally modified. A substantially purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a substantially purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

One embodiment of the invention provides one or more of the following polypeptides: SEQ ID NO:57 (whole BCV-1 polyprotein), SEQ ID NO:58 (BCV-1 Leader amino acid sequence), SEQ ID NO:35 (BCV-1 Leader* amino acid sequence), SEQ ID NO:59 (BCV-1 VP4 amino acid sequence), SEQ ID NO:60 (BCV-1 VP2 amino acid sequence), SEQ ID NO:61 (BCV-1 VP3 amino acid sequence), SEQ ID NO:62 (BCV-1 VP1 amino acid sequence), SEQ ID NO:63 (BCV-1 2A amino acid sequence), SEQ ID NO:64 (BCV-1 2B amino acid sequence), SEQ ID NO:65 (BCV-1 2C amino acid sequence), SEQ ID NO:66 (BCV-1 3AB amino acid sequence), SEQ ID NO:67 (BCV-1 3C amino acid sequence), SEQ ID NO:68 (BCV-1 3D amino acid sequence), SEQ ID NO:84 (BCV-2 polyprotein), SEQ ID NO:85 (BCV-2 Leader amino acid sequence), SEQ ID NO:86 (BCV-2 Leader* amino acid sequence), SEQ ID NO:87 (BCV-2 VP4 amino acid sequence), SEQ ID NO:88 (BCV-2 VP2 amino acid sequence), SEQ ID NO:89 (BCV-2 VP3 amino acid sequence), SEQ ID NO:90 (BCV-2 VP1 amino acid sequence), SEQ ID NO:91 (BCV-2 2A amino acid sequence), SEQ ID NO:92 (BCV-2 2B amino acid sequence), SEQ ID NO:93 (BCV-2 2C amino acid sequence), SEQ ID NO:94 (BCV-2 3AB amino acid sequence), SEQ ID NO:95 (BCV-2 3C amino acid sequence), SEQ ID NO:96 (BCV-2 3D partial amino acid sequence), SEQ ID NO:98 (consensus sequence of SEQ ID NO:57 and SEQ ID NO:84).

A polypeptide of the invention can comprise fragments of SEQ ID NOs:35, 57-68, 84-96, 98. A fragment can be for example, about 10, 15, 20, 30, 40, 50, 60, 70, 80, 90, 100, 200, 300, 400, 500, 600, 700, 800, 900, 1,000, 1,250, 1,500, 1750, 2,000, 2,250, 3,000, 4,000, 5,000, 6,000 or more amino acids (or any range or value between about 10 and about 6,000 amino acids). Additionally, a fragment can be, for example about 6,000, 5,000, 4,000, 3,000, 2,250, 2,000, 1,750, 1,500, 1,250, 1,000, 900, 800, 700, 600, 500, 400, 300, 200, 100, 90, 80, 70, 60, 50, 40, 30, 20, 15, 10 or less amino acids (or any range or value between about 6,000 and 10 amino acids). For example, a fragment may be between about 10-50, about 10-100, about 50-250, about 50-500, about 100-1,000 amino acids in length. In one embodiment of the invention a polypeptide of the invention or fragment thereof is an immunogenic polypeptide can elicit antibodies or other immune responses (e.g., T-cell responses of the immune system) that recognize epitopes of a polypeptide having SEQ ID NOs:35, 58-68, 84-96, 98 or fragments thereof.

Variant polypeptides have one or more conservative amino acid variations or other minor modifications and retain biological activity, i.e., are biologically functional equivalents. A biologically active equivalent has substantially equivalent function when compared to the corresponding wild-type polypeptide. In one embodiment of the invention a polypeptide has about 1, 2, 3, 4, 5, 10, 15, 20, 30, 40, 50, or less conservative amino acid substitutions. A variant polypeptide is at least about 80%, 85%, 90%, 91%, 92%, 93%, 94%, 95%, 96%, 97%, 98%, 99%, 99.5% or more identical to SEQ ID NO:35, 57-68, 84-96, 98 or a polypeptide comprising at least about 10, 15, 20, 30, 40, 50, 60, 70, 80, 90, 100, 200, 300, 400, 500, 600, 700, 800, 900, 1,000, 1,500, 2,000 or more contiguous amino acids of SEQ ID NO:35, 57-68, 84-96, 98.

SEQ ID NO:98 comprises a consensus polypeptide of SEQ ID NO:57 (BCV-1) and SEQ ID NO:84 (BCV-2). The alignment of BCV-1 SEQ ID NO:57 and BCV-2 SEQ ID NO:84 is shown below in the Sequences section. In the consensus sequence (SEQ ID NO:98) an X represents any amino acid or an absent amino acid. In one embodiment of the invention, the X represents either of the two amino acids (or an absent amino acid) that occur at that position in the alignment of BCV-1 SEQ ID NO:57 and BCV-2 SEQ ID NO:84 is shown below in the Sequences section. For example, in the alignment the amino acid at position 17 of BCV-1 is Y and the amino acid at position 17 of BCV-2 (which aligns with position 17 of BCV-1) is H. Therefore, in the consensus sequence the X for this position can be Y or H. This is also true for each smaller polynucleotide and fragment sequence. For example, polypeptide 2C (SEQ ID NO:65 for BCV-1 and SEQ ID NO:93 for BCV-2) have several X's within the consensus sequence. The X in the consensus sequence (SEQ ID NO:98) at position 1230 can be any amino acid. In another embodiment the X in the consensus sequence (SEQ ID NO:98) at 1230 can be P, which is the corresponding amino acid in BCV-1 2C (amino acid 41 of SEQ ID NO:65) or it can be S, which is the corresponding amino acid in BCV-2 2C (amino acid 41 of SEQ ID NO:93). Other examples in 2C include: the X at 1271 of consensus sequence SEQ ID NO:98, can be T (position 82 of BCV-1 SEQ ID NO:65) or S (position 82 of BCV-2 SEQ ID NO:93) and the X at position 1247 of consensus sequence SEQ ID NO:98, can be T (position 58 of BCV-1 SEQ ID NO:65) or I (position 58 of BCV-2 SEQ ID NO:93). The same analysis can be used to determine 2 alternate amino acids for each X in consensus sequence SEQ ID NO:98 for each full polypeptide, 5′UTR, leader, leader*, VP4, VP2, VP3, VP1, 2A, 2B, 2C, 3A, 3B, 3C, and 3D polypeptide.

In another embodiment, an X in consensus sequence SEQ ID NO:98 is a conservative amino acid substitution of one of the two amino acids present at that position in SEQ ID NO:57 or SEQ ID NO:84. For example, where an aliphatic amino acid (A, I, L, V) is present at a position at SEQ ID NO:57 or SEQ ID NO:84, then a different aliphatic amino acid can be substituted at that position. The same is true for aromatic amino acids (F, W, Y), amino acids with neutral side chains (N, C, Q, M, S, T), acidic amino acids (D, E), and basic amino acids (R, H, K). Other conservative substitutions include those within the following groups: (1) A, P, G, E, D, Q, N, S, T; (2) C, S, Y, T; (3) V, I, L, M, A, F; (4) K, R, H; and (5) F, Y, W, H.

A conservative substitution is one in which an amino acid is substituted for another amino acid that has similar properties, such that one skilled in the art of peptide chemistry would expect the secondary structure and hydropathic nature of the polypeptide to be substantially unchanged.

In another embodiment of the invention, an X in consensus sequence SEQ ID NO:98 can be substituted with strongly similar amino acids (marked with colon in alignment of SEQ ID NO:57 and SEQ ID NO:84 below) (e.g., the following amino acids can be substituted for each other M+V, L+V, K+N, F+I, M+L, T+S, D+E, R+K, N+E, F+Y, I+V, S+A, H+N, Y+H, N+D, Q+E, F+L, H+Q, L+I, A+T, R+Q). In another embodiment of the invention, an X in consensus sequence SEQ ID NO:98 can be substituted with weakly similar amino acids (marked with period in alignment of SEQ ID NO:57 and SEQ ID NO:84 below) (e.g., the following amino acids can be substituted for each other A+V, P+S, A+P, N+T, V+T, P+T, E+S, S+N, S+G, A+G, T+P, S+Q, C+S, V+A).

Variant polypeptides can generally be identified by modifying one of the polypeptide sequences of the invention, and evaluating the properties of the modified polypeptide to determine if it is a biological equivalent. A variant is a biological equivalent if it reacts substantially the same as a polypeptide of the invention in an assay such as an immunohistochemical assay, an enzyme-linked immunosorbent Assay (ELISA), a radioimmunoassay (RIA), immunoenzyme assay or a western blot assay, e.g. has 90-110% of the activity of the original polypeptide. In one embodiment, the assay is a competition assay wherein the biologically equivalent polypeptide is capable of reducing binding of the polypeptide of the invention to a corresponding reactive antigen or antibody by about 80, 95, 96, 97, 98, 99, 99.5 or 100%. An antibody that specifically binds a corresponding wild-type polypeptide also specifically binds the variant polypeptide.

A polypeptide of the invention can further comprise a signal (or leader) sequence that co-translationally or post-translationally directs transfer of the protein. The polypeptide can also comprise a linker or other sequence for ease of synthesis, purification or identification of the polypeptide (e.g., poly-His), or to enhance binding of the polypeptide to a solid support. For example, a polypeptide can be conjugated to an immunoglobulin Fc region or bovine serum albumin.

A polypeptide can be covalently or non-covalently linked to an amino acid sequence to which the polypeptide is not normally associated with in nature, i.e., a heterologous amino acid sequence. A heterologous amino acid sequence can be from a picornavirus, an organism other than BCV, a synthetic sequence, or a BCV sequence not usually located at the carboxy or amino terminus of a polypeptide of the invention. Additionally, a polypeptide can be covalently or non-covalently linked to compounds or molecules other than amino acids such as indicator reagents. A polypeptide can be covalently or non-covalently linked to an amino acid spacer, an amino acid linker, a signal sequence, a stop transfer sequence, a transmembrane domain, a protein purification ligand, or a combination thereof. A polypeptide can also be linked to a moiety (i.e., a functional group that can be a polypeptide or other compound) that enhances an immune response (e.g., cytokines such as IL-2), a moiety that facilitates purification (e.g., affinity tags such as a six-histidine tag, trpE, glutathione, maltose binding protein), or a moiety that facilitates polypeptide stability (e.g., polyethylene glycol; amino terminus protecting groups such as acetyl, propyl, succinyl, benzyl, benzyloxycarbonyl or t-butyloxycarbonyl; carboxyl terminus protecting groups such as amide, methylamide, and ethylamide). In one embodiment of the invention a protein purification ligand can be one or more C amino acid residues at, for example, the amino terminus or carboxy terminus of a polypeptide of the invention. An amino acid spacer is a sequence of amino acids that are not associated with a polypeptide of the invention in nature. An amino acid spacer can comprise about 1, 5, 10, 20, 100, or 1,000 amino acids.

If desired, a polypeptide of the invention can be part of a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and Staphylococcal protein A, or combinations thereof. Other amino acid sequences can be present at the C or N terminus of a polypeptide of the invention to form a fusion protein. More than one polypeptide of the invention can be present in a fusion protein. Fragments of polypeptides of the invention can be present in a fusion protein of the invention. A fusion protein of the invention can comprise one or more polypeptides of the invention, fragments thereof, or combinations thereof.

Polypeptides of the invention can be in a multimeric form. That is, a polypeptide can comprise one or more copies of a polypeptide of the invention or a combination thereof. A multimeric polypeptide can be a multiple antigen peptide (MAP). See e.g., Tam, J. Immunol. Methods, 196:17-32 (1996).

Polypeptides of the invention can comprise an antigenic determinant that is recognized by an antibody specific for BCV. The polypeptide can comprise one or more epitopes (i.e., antigenic determinants). An epitope can be a linear epitope, sequential epitope or a conformational epitope. Epitopes within a polypeptide of the invention can be identified by several methods. See, e.g., U.S. Pat. No. 4,554,101; Jameson & Wolf, CABIOS 4:181-186 (1988). For example, a polypeptide of the invention can be isolated and screened. A series of short peptides, which together span an entire polypeptide sequence, can be prepared by proteolytic cleavage. By starting with, for example, 30-mer polypeptide fragments, each fragment can be tested for the presence of epitopes recognized in an immunoassay. For example, in an immunoassay assay a BCV polypeptide, such as a 30-mer polypeptide fragment, is attached to a bead or solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is determined by detection of the bound antibody. Progressively smaller and overlapping fragments can then be tested from an identified 30-mer to map the epitope of interest.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, viral, yeast, plant, mammalian, and insect expression systems are available in the art and any such expression system can be used. Optionally, a polynucleotide encoding a polypeptide can be translated in a cell-free translation system. A polypeptide can also be chemically synthesized or obtained from cells infected with BCV.

### Host Cells and Expression Vectors

An expression vector is a nucleic acid construct, generated recombinantly or synthetically, with a set of nucleic acid elements that permit transcription of a particular nucleic acid in a host cell. The expression vector can be part of a plasmid, virus, or nucleic acid fragment. Typically, an expression vector includes a nucleic acid to be transcribed operably linked to a promoter.

A host cell can contain an expression vector and can support the replication or expression of the expression vector. Host cells can be prokaryotic cells such as E. coli, insect cells, or eukaryotic cells such as yeast, insect, amphibian, or mammalian cells such as CHO, HeLa, including, for example, cultured cells, explants, and cells in vivo.

### Antibodies

Antibodies of the invention are antibody molecules that specifically bind to a BCV polypeptide or variant polypeptide of the invention or fragment thereof. An antibody of the invention can be specific for a BCV polypeptide or a variant BCV polypeptide or a combination thereof, for example, an antibody specific for one or more of SEQ ID NOs:35, 57-68, 84-96, 98 or fragments thereof. One of skill in the art can easily determine if an antibody is specific for a BCV polypeptide using assays described herein. An antibody of the invention can be a polyclonal antibody, a monoclonal antibody, a single chain antibody (scFv), or an antigen binding fragment of an antibody. Antigen-binding fragments of antibodies are a portion of an intact antibody comprising the antigen binding site or variable region of an intact antibody, wherein the portion is free of the constant heavy chain domains of the Fc region of the intact antibody. Examples of antigen binding antibody fragments include Fab, Fab′, Fab′-SH, F(ab′)₂ and Fᵥ fragments.

An antibody of the invention can be any antibody class, including for example, IgG, IgM, IgA, IgD and IgE. An antibody or fragment thereof binds to an epitope of a polypeptide of the invention. An antibody can be made in vivo in suitable laboratory animals or in vitro using recombinant DNA techniques. Means for preparing and characterizing antibodies are well known in the art. See, e.g., Dean, Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68 (1992). For example, polyclonal antibodies can be produced by administering a polypeptide of the invention to an animal, such as a human or other primate, mouse, rat, rabbit, guinea pig, goat, pig, dog, cow, sheep, donkey, or horse. Serum from the immunized animal is collected and the antibodies may be purified from the plasma by, for example, precipitation with ammonium sulfate, followed by chromatography, such as affinity chromatography. Techniques for producing and processing polyclonal antibodies are known in the art.

“Specifically binds” or “specific for” means that a first antigen, e.g., a BCV polypeptide, recognizes and binds to an antibody of the invention with greater affinity than to other, non-specific molecules. A non-specific molecule is an antigen that shares no common epitope with the first antigen. In a preferred embodiment of the invention a non-specific molecule is not derived from BCV or picornaviruses. For example, an antibody raised against a first antigen (e.g., a polypeptide) to which it binds more efficiently than to a non-specific antigen can be described as specifically binding to the first antigen. In one embodiment, an antibody or antigen-binding fragment thereof specifically binds to a polypeptide of SEQ ID NOs:35, 57-68, 84-96, 98 or fragments thereof when it binds with a binding affinity Kₐ of 10⁷ l/mol or more. Specific binding can be tested using, for example, an enzyme-linked immunosorbant assay (ELISA), a bead-based multiplex fluorescent immunoassay (MFI), a radioimmunoassay (RIA), or a western blot assay using methodology well known in the art.

Antibodies of the invention include antibodies and antigen binding fragments thereof that (a) compete with a reference antibody for binding to SEQ ID NOs: 35, 57-68, 84-96, 98 or antigen binding fragments thereof; (b) binds to the same epitope of SEQ ID NOs: 35, 57-68, 84-96, 98 or antigen binding fragments thereof as a reference antibody; (c) binds to SEQ ID NOs:35, 57-68, 84-96, 98 or antigen binding fragments thereof with substantially the same K_{d} as a reference antibody; and/or (d) binds to SEQ ID NOs:35, 57-68, 84-96, 98 or fragments thereof with substantially the same off rate as a reference antibody, wherein the reference antibody is an antibody or antigen-binding fragment thereof that specifically binds to a polypeptide of SEQ ID NOs:35, 57-68, 84-96, 98 or antigen binding fragments thereof with a binding affinity Kₐ of 10⁷ l/mol or more.

Additionally, monoclonal antibodies directed against epitopes present on a polypeptide of the invention can also be readily produced. For example, normal B cells from a mammal, such as a mouse, which was immunized with a polypeptide of the invention can be fused with, for example, HAT-sensitive mouse myeloma cells to produce hybridomas. Hybridomas producing BCV-specific antibodies can be identified using RIA or ELISA and isolated by cloning in semi-solid agar or by limiting dilution. Clones producing BCV-specific antibodies are isolated by another round of screening. Monoclonal antibodies can be screened for specificity using standard techniques, for example, by binding a polypeptide of the invention to a microtiter plate and measuring binding of the monoclonal antibody by an ELISA assay. Techniques for producing and processing monoclonal antibodies are known in the art. See e.g., Kohler & Milstein, Nature, 256:495 (1975). Particular isotypes of a monoclonal antibody can be prepared directly, by selecting from the initial fusion, or prepared secondarily, from a parental hybridoma secreting a monoclonal antibody of a different isotype by using a sib selection technique to isolate class-switch variants. See Steplewski et al., P.N.A.S. U.S.A. 82:8653 1985; Spria et al., J. Immunolog. Meth. 74:307, 1984. Monoclonal antibodies of the invention can also be recombinant monoclonal antibodies. See, e.g., U.S. Pat. No. 4,474,893; U.S. Pat. No. 4,816,567. Antibodies of the invention can also be chemically constructed. See, e.g., U.S. Pat. No. 4,676,980.

Antibodies of the invention can be chimeric (see, e.g., U.S. Pat. No. 5,482,856) or humanized (see, e.g., Jones et al., Nature 321:522 (1986); Reichmann et al., Nature 332:323 (1988); Presta, Curr. Op. Struct. Biol. 2:593 (1992)). An antibody of the invention can be “murinized,” which is an antibody comprising one or more CDRs from an animal antibody, the antibody having being modified in such a way so as to be less immunogenic in a mouse than the parental animal antibody. An animal antibody can be murinized using a number of methodologies, including chimeric antibody production, CDR grafting (also called reshaping), and antibody resurfacing. An antibody can also be “ratinized” (similar to murinized antibodies), rat, mouse, or human antibodies. Human antibodies can be made by, for example, direct immortalization, phage display, transgenic mice, or a Trimera methodology, see e.g., Reisener et al., Trends Biotechnol. 16:242-246 (1998).

Antibodies that specifically bind BCV antigens are particularly useful for detecting the presence of BCV antigens in a sample, such as a serum, blood, plasma, urine, fecal, tissue, cell, or saliva sample from a BCV-infected animal.

Antibodies of the invention can be used to isolate BCV organisms or antigens by immunoaffinity columns. The antibodies can be affixed to a solid support by, for example, absorption or by covalent linkage so that the antibodies retain their immunoselective activity. Optionally, spacer groups can be included so that the antigen binding site of the antibody remains accessible. The immobilized antibodies can then be used to bind BCV organisms or BCV antigens from a sample, such as a biological sample including saliva, serum, sputum, blood, urine, feces, cerebrospinal fluid, amniotic fluid, wound exudate, cells, or tissue, or an environmental or laboratory sample. The bound BCV organisms or BCV antigens are recovered from the column matrix by, for example, a change in pH.

Antibodies of the invention can also be used in immunolocalization studies to analyze the presence and distribution of a polypeptide of the invention during various cellular events or physiological conditions. Antibodies can also be used to identify molecules involved in passive immunization and to identify molecules involved in the biosynthesis of non-protein antigens. Identification of such molecules can be useful in vaccine development. Antibodies can be detected and/or quantified using for example, direct binding assays such as RIA, ELISA, or western blot assays.

### Detection, Diagnosis and Quantification

Detection and quantification of a BCV or BCV polynucleotides of the invention in a sample can be done using any method known in the art, including, for example, direct sequencing, hybridization with probes, gel electrophoresis, transcription mediated amplification (TMA) (e.g., U.S. Pat. No. 5,399,491), polymerase chain reaction (PCR), reverse transcriptase PCR (RT-PCR), quantitative PCR, replicase mediated amplification, ligase chain reaction (LCR), competitive quantitative PCR (QPCR), real-time quantitative PCR, self-sustained sequence replication, strand displacement amplification, branched DNA signal amplification, nested PCR, in situ hybridization, multiplex PCR, Rolling Circle Amplification (RCA), Q-beta-replicase system, and mass spectrometry. These methods can use heterogeneous or homogeneous formats, labels or no labels, and can detect or detect and quantify. The quantification can be semi-quantitative or fully quantitative.

In one embodiment, a BCV polynucleotide can be detected by amplifying polynucleotides of a sample suspected of containing a Boone cardiovirus polynucleotide with at least one primer (e.g., 1, 2, 3, 4, or more primers) that hybridizes to at least about 8, 10, 15, 20, 30, 40 or more contiguous nucleic acids of SEQ ID NO:5, 42-56, 69-83, 97 or a complement thereof, to produce an amplification product. The presence of the amplification product is then detected, thereby detecting the presence of the Boone cardiovirus polynucleotide. In another embodiment, a BCV polynucleotide can be detected by contacting a sample with one or more isolated nucleic acid probes comprising about 8, 10, 15, 20, 30, 40, 50, 60, 70, 80, 90, 100 or more contiguous nucleic acids of SEQ ID NO:5, 42-56, 69-83, 97; and detecting the presence of hybridized probe/Boone cardiovirus nucleic acid complexes, wherein the presence of hybridized probe/Boone cardiovirus nucleic acid complexes indicates the presence of Boone cardiovirus in the sample.

A sample includes, for example, purified nucleic acids, unpurified nucleic acids, cells, cellular extract, tissue, organ fluid, bodily fluid, tissue sections, specimens, aspirates, bone marrow aspirates, tissue biopsies, tissue swabs, fine needle aspirates, skin biopsies, blood, serum, lymph fluid, cerebrospinal fluid, seminal fluid, stools, or urine from a mammal such as a human, rat, mouse, bovine, equine, canine, or feline. A sample can also comprise an environmental sample or a laboratory sample. The test sample can be untreated, precipitated, fractionated, separated, diluted, concentrated, or purified.

BCV target nucleic acids can be separated from non-homologous nucleic acids using capture polynucleotides immobilized, for example, on a solid support. The capture polynucleotides are specific for BCV of the invention (e.g., 10, 15, 20, 30, 40, 50, 60, 70, 80, 90, 100, or more contiguous nucleotides of SEQ ID NO:5, 42-56, 69-83, 97 or complements thereof). The separated target nucleic acids can then be detected, for example, by the use of polynucleotide probes (e.g., 10, 15, 20, 30, 40, 50, 60, 70, 80, 90, 100, or more contiguous nucleotides of SEQ ID NO:5, 42-56, 69-83, 97 or complements thereof). More than one probe can be used.

In one embodiment of the invention a sample is contacted with a solid support in association with capture polynucleotides. The capture polynucleotides can be associated with the solid support by, for example, covalent binding of the capture polynucleotide to the solid support, by affinity association, hydrogen binding, or nonspecific association.

A capture polynucleotide can be immobilized to the solid support using any method known in the art. For example, the polynucleotide can be immobilized to the solid support by attachment of the 3′ or 5′ terminal nucleotide of the probe to the solid support. Alternatively, the capture polynucleotide can be immobilized to the solid support by a linker. A wide variety of linkers are known in the art that can be used to attach the polynucleotide probe to the solid support. The linker can be formed of any compound that does not significantly interfere with the hybridization of the target sequence to the capture polynucleotide associated with the solid support.

A solid support for any assay of the invention can be, for example, particulate nitrocellulose, nitrocellulose, materials impregnated with magnetic particles or the like, beads or particles, polystyrene beads, controlled pore glass, glass plates, polystyrene, avidin-coated polystyrene beads, cellulose, nylon, acrylamide gel and activated dextran.

The solid support with immobilized capture polynucleotides is brought into contact with a sample under hybridizing conditions. The capture polynucleotides hybridize to the target polynucleotides present in the sample.

The solid support can then be separated from the sample, for example, by filtering, washing, passing through a column, or by magnetic means, depending on the type of solid support. The separation of the solid support from the sample preferably removes at least about 70%, more preferably about 90% and, most preferably, at least about 95% or more of the non-target nucleic acids and other debris present in the sample.

In one embodiment of the invention the sequence of a BCV polynucleotide (e.g., 5′UTR, Leader, Leader*, VP4, VP2, VP3, VP1, 2A, 2B, 2C, 3A, 3B, 3C, 3D, 3′UTR) or fragment or complement thereof can be used to detect the presence or absence of BCV in a sample. For example, a sample can be contacted with a probe comprising SEQ ID NOs:5, 42-56, 69-83, 97 or a probe comprising 10, 15, 20, 30, 40, 50, 60, 70, 80, 90, 100 or more contiguous nucleic acids of SEQ ID NOs:5, 42-56, 69-83, 97 or complements thereof. The probe can comprise a label, such as a fluorescent label. The presence or absence of hybridized nucleic acid probe/BCV nucleic acid complexes is detected. The presence of hybridized probe/BCV nucleic acid complexes indicates the presence of BCV of the invention in the sample. The quantity of hybridized nucleic acid probe/BCV nucleic acid complexes can be determined.

Another embodiment of the invention provides a method of detecting a nucleic acid molecule of a BCV of the invention in a sample. Nucleic acid molecules of BCV are amplified using a first amplification primer and a second amplification primer. The amplified nucleic acid molecules are detected using any methodology known in the art. Amplification products can be assayed in a variety of ways, including size analysis, restriction digestion followed by size analysis, detecting specific tagged oligonucleotide primers in the reaction products, allele-specific oligonucleotide (ASO) hybridization, sequencing, and the like. The quantity of the amplified BCV nucleic acid molecules can also be determined. The amplification primers can further comprise a label, such as a fluorescent moiety.

An internal control (IC) or an internal standard can be added to an amplification reaction serve as a control for target capture and amplification. Preferably, the IC includes a sequence that differs from the target sequences, is capable of hybridizing with the capture polynucleotides used for separating the nucleic acids specific for the BCV from the sample, and is capable of amplification by the primers used to amplify the BCV nucleic acids.

Another embodiment of the invention provides a method for detecting a BCV of the invention in a sample. A quantitative real-time PCR reaction can be performed with reagents comprising nucleic acid molecules of BCV, a dual-fluorescently labeled nucleic acid hybridization probe, and a set or sets of species-specific primers (i.e., one forward and one reverse primer). The fluorescent labels can be detected and read during the PCR reaction. The dual-fluorescently labeled probe can be labeled with a reporter fluorescent dye and a quencher fluorescent dye. See, Quantitation of DNA/RNA Using Real-Time PCR Detection, Perkin Elmer Applied Biosystems (1999); PCR Protocols (Academic Press New York, 1989). By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed.

Other embodiments of the invention provide methods of diagnosis of infection with BCV. Another embodiment of the invention provides methods for screening a subject for an infection with a BCV. A polynucleotide comprising SEQ ID NOs:5, 42-56, 69-83, 97 or 10, 15, 20, 30, 40, 50, 60, 70, 80, 90, 100 or more contiguous nucleic acids of SEQ ID NOs:5, 42-56, 69-83, 97 or complements thereof can be used to detect BCV polynucleotides in a sample. If the BCV polynucleotide is detected, then the subject has an infection with a BCV of the invention. Alternatively, a polynucleotide comprising SEQ ID NOs:5, 42-56, 69-83, 97 or 10, 20, 30, 40, 50, 60, 70, 80, 90, 100 or more contiguous nucleic acids of SEQ ID NOs:5, 42-56, 69-83, 97 or complements thereof can be detected in a sample obtained from the subject to provide a first value. A polynucleotide comprising SEQ ID NOs: 5, 42-56, 69-83, 97 or 10, 20, 30, 40, 50, 60, 70, 80, 90, 100 or more contiguous nucleic acids of SEQ ID NOs:5, 42-56, 69-83, 97 or complements thereof can be detected in a similar biological sample obtained from a disease-free subject to provide a second value. The first value can be compared with the second value, wherein a greater first value relative to the second value is indicative of the subject having an infection with the BCV.

One embodiment of the invention provides a method of detecting Boone cardiovirus polypeptides in a sample. The method comprises contacting the sample suspected of containing Boone cardiovirus polypeptides with an antibody or antigen binding fragment thereof of the invention to form Boone cardiovirus polypeptide/antibody complexes. The presence of the Boone cardiovirus polypeptide/antibody complexes are detected, thereby detecting the presence of the Boone cardiovirus polypeptides. Polypeptide/antibody complexes can be detected by any method known in the art, enzyme-linked immunosorbent assay (ELISA), multiplex fluorescent immunoassay (MFI or MFIA), radioimmunoassay (RIA), sandwich assay, Western blotting, immunoblotting analysis, an immunohistochemistry method, immunofluorescence assay, or a combination thereof.

Another embodiment of the invention provides a method of detecting antibodies that specifically bind a BCV polypeptide in a test sample. The method comprises contacting one or more of the purified polypeptides or polypeptide fragments of the invention (e.g., VP1, VP2, VP3, 2A-C, and 3A-D, although any polypeptide or fragment can be used) with the test sample, under conditions that allow polypeptide/antibody complexes to form. The polypeptide/antibody complexes are detected, wherein the detection of the polypeptide/antibody complexes is an indication that antibodies specific for a BCV polypeptide are present in the test sample.

An immunoassay for a BCV antigen can utilize one antibody or several antibodies. An immunoassay for a BCV antigen can use, for example, a monoclonal antibody specific for a BCV epitope, a combination of monoclonal antibodies specific for epitopes of one BCV polypeptide, monoclonal antibodies specific for epitopes of different BCV polypeptides, polyclonal antibodies specific for the same BCV antigen, polyclonal antibodies specific for different BCV antigens, a combination of monoclonal and polyclonal antibodies, or serum from an a human or animal. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels.

Antibodies of the invention or fragments thereof can be bound to a support and used to detect the presence of BCV antigens, just as polypeptides of the invention can be bound to a support and used to detect the presence of antibodies specific for BCV polypeptides. Supports include, for example, glass, polystyrene, polypropylene, polyethylene, dextran, nylon, amylases, natural and modified celluloses, polyacrylamides, agaroses and magletite.

In one embodiment methods of the invention comprise contacting one or more polypeptides of the invention with a test sample under conditions that allow polypeptide/antibody complexes, i.e., immunocomplexes, to form. That is, polypeptides of the invention specifically bind to antibodies specific for BCV antigens located in the sample. In one embodiment of the invention one or more polypeptides of the invention (e.g., SEQ ID NO:35, 57-68, 84-96, 98 or fragments thereof) specifically bind to antibodies that are specific for BCV antigens and do not specifically bind to other picornavirus antigens. One of skill in the art is familiar with assays and conditions that are used to detect antibody/polypeptide complex binding. The formation of a complex between polypeptides and anti-BCV antibodies in the sample is detected. The formation of antibody/polypeptide complexes is an indication that BCV polypeptides are present in the sample. The lack of detection of the polypeptide/antibody complexes is an indication that BCV polypeptides are not present in the sample.

Antibodies of the invention can be used in a method of the diagnosis of BCV infection by obtaining a test sample from, e.g., a human or animal suspected of having a BCV infection. The test sample is contacted with antibodies of the invention under conditions enabling the formation of antibody-antigen complexes (i.e., immunocomplexes). One of skill in the art is aware of conditions that enable and are appropriate for formation of antigen/antibody complexes. The amount of antibody-antigen complexes can be determined by methodology known in the art. A level that is higher than that formed in a control sample indicates a BCV infection. A control sample is a sample that does not comprise any BCV polypeptides or antibodies specific for BCV. In one embodiment of the invention the control contains picornavirus polypeptides or antibodies. Alternatively, a polypeptide or fragment thereof of the invention can be contacted with a test sample. Antibodies specific for BCV in a positive test sample will form antigen-antibody complexes under suitable conditions. The amount of antibody-antigen complexes can be determined by methods known in the art.

In one embodiment of the invention, BCV infection can be detected in a subject. A biological sample is obtained from the subject. One or more purified polypeptides comprising SEQ ID NOs:35, 57-68, 84-96, 98 or other polypeptides of the invention are contacted with the biological sample under conditions that allow polypeptide/antibody complexes to form. The polypeptide/antibody complexes are detected. The detection of the polypeptide/antibody complexes is an indication that the mammal has a BCV infection. The lack of detection of the polypeptide/antibody complexes is an indication that the mammal does not have a BCV infection.

In one embodiment of the invention, the polypeptide/antibody complex is detected when an indicator reagent, such as an enzyme conjugate, which is bound to the antibody, catalyzes a detectable reaction. Optionally, an indicator reagent comprising a signal generating compound can be applied to the polypeptide/antibody complex under conditions that allow formation of a polypeptide/antibody/indicator complex. The polypeptide/antibody/indicator complex is detected. Optionally, the polypeptide or antibody can be labeled with an indicator reagent prior to the formation of a polypeptide/antibody complex. The method can optionally comprise a positive or negative control.

In one embodiment of the invention, one or more antibodies of the invention are attached to a solid phase or substrate. A test sample potentially comprising a polypeptide of the invention is added to the substrate. One or more antibodies that specifically bind polypeptides of the invention are added. The antibodies can be the same antibodies used on the solid phase or can be from a different source or species and can be linked to an indicator reagent, such as an enzyme conjugate. Wash steps can be performed prior to each addition. A chromophore or enzyme substrate is added and color is allowed to develop. The color reaction is stopped and the color can be quantified using, for example, a spectrophotometer.

In another embodiment of the invention, one or more antibodies of the invention are attached to a solid phase or substrate. A test sample potentially comprising a polypeptide of the invention is added to the substrate. Second anti-species antibodies that specifically bind polypeptides of the invention are added. These second antibodies are from a different species than the solid phase antibodies. Third anti-species antibodies are added that specifically bind the second antibodies and that do not specifically bind the solid phase antibodies are added. The third antibodies can comprise an indicator reagent such as an enzyme conjugate. Wash steps can be performed prior to each addition. A chromophore or enzyme substrate is added and color is allowed to develop. The color reaction is stopped and the color can be quantified using, for example, a spectrophotometer.

Assays of the invention include, but are not limited to those based on competition, direct reaction or sandwich-type assays, including, but not limited to enzyme linked immunosorbent assay (ELISA), multiplex fluorescent immunoassay (MFI or MFIA) western blot, IFA, radioimmunoassay (RIA), western blot, hemagglutination (HA), fluorescence polarization immunoassay (FPIA), and microtiter plate assays (any assay done in one or more wells of a microtiter plate). One assay of the invention comprises a reversible flow chromatographic binding assay, for example a SNAP® assay. See U.S. Pat. No. 5,726,010.

Assays can use solid phases or substrates or can be performed by immunoprecipitation or any other methods that do not utilize solid phases. Where a solid phase or substrate is used, one or more polypeptides of the invention are directly or indirectly attached to a solid support or a substrate such as a microtiter well, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). In one embodiment of the invention a substrate is sintered, fine particles of polyethylene, commonly known as porous polyethylene, for example, 10-15 micron porous polyethylene from Chromex Corporation (Albuquerque, N. Mex.). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In one type of assay format, one or more polypeptides can be coated on a solid phase or substrate. A test sample suspected of containing an anti-BCV antibody or antigen-binding fragment thereof is incubated with an indicator reagent comprising a signal generating compound conjugated to an antibody or antigen-binding antibody fragment specific for BCV for a time and under conditions sufficient to form antigen/antibody complexes of either antibodies of the test sample to the polypeptides of the solid phase or the indicator reagent compound conjugated to an antibody specific for BCV to the polypeptides of the solid phase. The reduction in binding of the indicator reagent conjugated to an anti-BCV and/or anti-BCV antibody to the solid phase can be quantitatively measured. A measurable reduction in the signal compared to the signal generated from a confirmed negative BCV test sample indicates the presence of anti-BCV antibody in the test sample. This type of assay can quantitate the amount of anti-BCV antibodies in a test sample.

In another type of assay format, one or more polypeptides of the invention are coated onto a support or substrate. A polypeptide of the invention is conjugated to an indicator reagent and added to a test sample. This mixture is applied to the support or substrate. If antibodies specific for BCV are present in the test sample they will bind the one or more polypeptides conjugated to an indicator reagent and to the one or more polypeptides immobilized on the support. The polypeptide/antibody/indicator complex can then be detected. This type of assay may quantitate the amount of BCV antibodies in a test sample.

In another type of assay format, one or more polypeptides of the invention are coated onto a support or substrate. The test sample is applied to the support or substrate and incubated. Unbound components from the sample are washed away by washing the solid support with a wash solution. If BCV-specific antibodies are present in the test sample, they will bind to the polypeptide coated on the solid phase. This polypeptide/antibody complex can be detected using a second species-specific antibody that is conjugated to an indicator reagent. The polypeptide/antibody/anti-species antibody indicator complex can then be detected. This type of assay can quantitate the amount of anti-BCV antibodies in a test sample.

The formation of a polypeptide/antibody complex or a polypeptide/antibody/indicator complex can be detected by e.g., radiometric, colorimetric, fluorometric, size-separation, or precipitation methods. Optionally, detection of a polypeptide/antibody complex is by the addition of a secondary antibody that is coupled to an indicator reagent comprising a signal generating compound. Indicator reagents comprising signal generating compounds (labels) associated with a polypeptide/antibody complex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

Formation of the complex is indicative of the presence of anti-BCV antibodies in a test sample. Therefore, the methods of the invention can be used to diagnose BCV infection in a mammal.

The methods of the invention can also indicate the amount or quantity of anti-BCV antibodies in a test sample. With many indicator reagents, such as enzyme conjugates, the amount of antibody present is proportional to the signal generated. Depending upon the type of test sample, it can be diluted with a suitable buffer reagent, concentrated, or contacted with a solid phase without any manipulation. For example, it usually is preferred to test serum or plasma samples that previously have been diluted, or concentrated specimens such as urine, in order to determine the presence and/or amount of antibody present.

All assays for BCV polypeptides, polynucleotides, and antibodies specific for BCV can be combined with one or more assays for one or more other viruses, bacteria, fungi, or protozoans. For example, the invention includes a panel of PCR primers comprising one or more sets of primers that amplify BCV polynucleotides or one or more probes specific for BCV polynucleotides and one or more sets of PCR primers that amplify one or more polynucleotides from other viruses, bacteria, fungi or protozoans or one or more probes specific for one or more polynucleotides from other viruses, bacteria, fungi or protozoans. Also included in the invention is a panel of antibodies that are specific for one or more BCV polypeptides and one or more antibodies that are specific for one or more polypeptides from other viruses, bacteria, fungi or protozoans. Additionally, the invention comprises a panel of BCV polypeptides that specifically bind a BCV antibody and one or more polypeptides that are specific for one or more antibodies from other viruses, bacteria, fungi or protozoans. These three types of panels or portions thereof can be combined into one panel. The detection of each organism can be done on separate portions of an assay device (e.g., in separate microtiter wells or on separate portions of a solid support) or the detection of more than one organism can done on one portion of an assay device (e.g., more than one detection reaction occurs in, e.g., one microtiter well or one portion of a solid support). Examples of other organisms that can be detected in a panel or in an assay run as the same time as a BCV assay include, e.g., RCV (rat coronavirus), NS1 (generic Parvovirus, RPV (rat parvovirus), RMV (rat minute virus), KRV (kilham rat virus), Toolan's H-1 virus, RTV (rat theilovirus), Sendai virus, PVM (pneumonia virus of mice), Mycoplasma pulmonis, REO3 (reovirus), LCMV (lymphocytic choriomeningitis virus), GARB (cilia-associated respiratory bacillus), Hataan virus, Clostridium piliforme, MAD1 (mouse adenovirus 1), MAD2 (mouse adenovirus 2), Encephalitozoon cuniculi, and IDIR (rat rotavirus). Regents for detecting organisms other than Boone cardiovirus are well known to those of skill in the art, see, e.g., IDEXX RADIL™ testing.

### Kits

The above-described assay reagents, including primers, probes, solid supports, as well as other detection reagents, can be provided in kits, with suitable instructions and other necessary reagents, in order to conduct, for example, the assays as described above. A kit can contain, in separate containers, the combination of primers and probes (either already bound to a solid support or separate with reagents for binding them to the support), control formulations (positive and/or negative), labeled reagents and signal generating reagents (e.g., enzyme substrate) if the label does not generate a signal directly. Instructions for carrying out the assay can also be included in the kit. The kit can also contain, depending on the particular assay used, other packaged reagents and materials (i.e., wash buffers and the like). Standard assays, such as those described above, can be conducted using these kits.

A kit can comprise, for example, one or more nucleic acid molecules having a sequence comprising SEQ ID NO:5, 42-56, 69-83, 97; 10, 15, 20, 30, 40, 50, 60, 70, 80, 90, 100 or more contiguous nucleic acids of SEQ ID NOs:5, 42-56, 69-83, 97, complements thereof or combinations thereof, and a polymerase and one or more buffers. The one or more nucleic acid molecules can comprise one or more labels or tags. The label can be a fluorescent moiety.

The invention further comprises assay kits for detecting anti-BCV antibodies, anti-BCV antibody fragments, and/or BCV polypeptides in a sample. A kit comprises one or more polypeptides of the invention and means for determining binding of the polypeptide to anti-BCV antibodies or antigen-binding antibody fragments in the sample. A kit can also comprise one or more antibodies or antigen-binding antibody fragments of the invention and means for determining binding of the antibodies or antigen-binding antibody fragments to BCV polypeptides in the sample. A kit can comprise a device containing one or more polypeptides or antibodies of the invention and instructions for use of the one or more polypeptides or antibodies for, e.g., the identification of BCV infection in a mammal. The kit can also comprise packaging material comprising a label that indicates that the one or more polypeptides or antibodies of the kit can be used for the identification of BCV infection. Other components such as buffers, controls, and the like, known to those of ordinary skill in art, can be included in such test kits. A kit can further comprise one or more polynucleotides, one or more substantially purified polypeptides, one or more antibodies or antigen binding fragments that can detect one or more viruses, bacteria, fungi or protozoans other than Boone cardiovirus.

The polypeptides, antibodies, assays, and kits of the invention are useful, for example, in the diagnosis of individual cases of BCV infection in a mammal, as well as epidemiological studies of BCV outbreaks.

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1

### Direct PCR and Sequencing

Picornavirus primers designed to the 5′ untranslated region (5′ UTR) were used to screen rat fecal samples from a variety of sources submitted to IDEXX-RADIL for routine diagnostic testing (20). To isolate RNA half to one whole rat fecal pellet were homogenized in Buffer RLT plus 1% β-mercaptoethanol using 5 mm stainless steel ball bearings and a TissueLyser (Qiagen, Valencia, Calif.). Samples were homogenized at 30 hertz (Hz) for 30 seconds and the lysates were centrifuged at 1300×g for 5 minutes. RNA was purified from the resulting supernatant using standard protocols on the BioRobot M48 Workstation with the MagAttract® RNA Tissue M48 Kit (Qiagen). The standard protocol for OneStep RT-PCR Kit plus Q Solution (Qiagen) was used for amplification of RNA: 10 μl RNA, 10 mM each dNTP, 20 mM sense and antisense primers; in a total reaction volume of 50 μl. Reverse transcription was performed at 50° C. for 45 minutes followed by 95° C. for 15 minutes to activate the DNA polymerase. DNA was amplified in 40 cycles of 94° C. for 30 seconds, 61° C. for 35 seconds, 72° C. for 35 seconds; followed by a final extension of 72° C. for 5 minutes. A 15 μl aliquot of the PCR products was run on a 3% agarose gel (Bio-Rad Laboratories, Hercules, Calif.). Products were cloned using a TOPO® TA Cloning® kit (Invitrogen, Carlsbad, Calif.) and sequenced by the University of Missouri DNA Core. NCBI blast analysis was performed on the sequencing results to confirm the presence of a picornavirus.

### Example 2

### Sample Preparation

Utilizing initial sequence information, an in-house colony of rats was determined to be naturally infected with the new picornavirus. A volume of 50 ml of fresh fecal pellets was collected and homogenized in sterile PBS using a homogenizer (Omni, Waterbury, Conn.). The lysate was centrifuged at 15,000×g for 20 minutes to pellet cellular debris from the sample; this was repeated once. To concentrate virus in the supernatant the sample was centrifuged at 100,000×g for 2 hours. The resulting pellet was re-suspended in 500 μl of 50 mM Tris, 50 mM MgCl₂, 0.1 mg/ml BSA, at pH 8. The re-suspension was sonicated at 16 hz to break up and solubilize proteins prior to centrifugation at 15,000×g for 15 minutes to pellet the remaining insoluble proteins. The resulting sample was digested with 250 units of the Benzonase® endonuclease (Novagen, Madison, Wis.) for 24 hours at 4° C. with gentle agitation to digest any free DNA and RNA in the sample. Benzonase® was inactivated with proteinase K and RNA was extracted using a standard TRIZOL (Invitrogen) protocol with glycogen acting as an RNA carrier. RNA concentration and purity were determined by evaluating the A260 and A280 on a spectrophotometer. To confirm viral RNA from the novel virus was present in the final sample, a RT-PCR using the BCV primers was performed.

### Example 3

### Primer Walking and Sequencing

To sequence the full-length virus the SMARTer™ RACE Amplification kit (Clontech, Mountain View, Calif.) was used. For the primary and nested 3′ race reaction viral-specific sense primer 5′-CCCTTGAGAGCGGTGGTACCC-3′ (SEQ ID NO:1) and 5′-CCCTGAAGGTACCCGTGTTGAAATCGC-3′ (SEQ ID NO:2) were used, respectively. For primary and nested 5′ Race PCR the viral specific anti-sense primers 5′-GCGATTTCAACACGGGTACCTTCAGGGC-3′ (SEQ ID NO:3) and 5′-CGGGTACCTTCAGGGCATCCTTAGCCG-3′ (SEQ ID NO:4) were used, respectively. The 3′ race product was expected to be approximately 7 kb in size and was visualized by running the reaction on a 1% TBE agarose gel and staining with crystal violet. The resulting products were excised from the gel and DNA purified and cloned according to the directions in the TOPO® XL cloning kit (Invitrogen). The 5′ race products were expected to 1 kb or less and were run on 1% precast agarose gels containing ethidium bromide (Bio-Rad Laboratories) and visualized using ultraviolet light. Bands were purified with the Wizard® SV Gel and PCR Clean-Up System (Promega, Madison, Wis.). DNA was cloned using TOPO® TA Cloning (Invitrogen, Carlsbad, Calif.). Plasmid DNA from both 3′ and 5′ race clones were purified using the Wizard® Plus SV Minipreps DNA Purification System (Promega, Madison, Wis.) and submitted to SeqWright (Houston, Tex.) for double strand sequence walking using florescent dye-terminator chemistry on an ABI™ Prism 3730xl DNA sequencer for 4× redundant coverage. NCBI blast analysis was performed on both nucleotide and translated protein sequence to determine closest viral identity.

### Example 4

### Phylogenetic Analysis

For amino acid analysis, proteins and ORFs were predicted using ORF Finder (National Center for Biotechnology Information). Nucleotide sequences for the following picornaviruses were downloaded from GenBank and aligned by CLUSTALW: Foot and mouth disease virus (FMDV), AF308157; Echovirus 5, AF083069; Human rhinovirus 1B (HRV-1B), D0023999; Porcine enterovirus 8, AF406813; Human hepatitis A (HAV), M20273; Simian hepatitis A, D00924; Ljungan virus (LV), AF327921; Human parechovirus 1 (HPeV-1), L02971, Human parechovirus 2 (HPeV-2), AJ005695; Cosavirus (hCoSV-B1), FJ438907; Senecavirus (SW), DQ641257; Mouse mosavirus, JF973687; Mouse kobuvirus (MKV-1), JF755427; Human klassevirus, NC_{—}012986; Saffold virus (SAFV) prototype, NC_{—}009448; SAFV Canadian strain 112051-06, JF813004; SAFV, FM207487; Thera virus (RTV-1), EU542581; Vilyuisk human encephalomyelitis (VHEV), M94868, M80888, and EU723237; Theiler's murine encephalomyelitis (TMEV) GDVII, X56019; TMEV-DA, M20301; Mengo, L22089, and Encephalomyelitis (EMCV), NC_{—}001479. Phylogenetic (neighbor joining) trees were generated with MEGA5 (28). Branch confidence was determined with bootstrap resampling of 1,000 pseudoreplicates. Evolutionary distances were computed using the p-distance method. Genome similarity plots were generated from aligned sequences using SimPlot version 3.5.1 with the parameters: 300 by window, 10 by step, and Kimura 2-parameter distance model (17). Sequence identity matrixes were generated in BioEdit using aligned amino acid sequences (11). The whole genome sequence of BCV-1 has been deposited in the GenBank database (accession number JQ864242) (SEQ ID NO:5). The partial genome sequence of BCV-2 is shown in SEQ ID NO:69 (GenBank accession number JX683808). The invention includes isolated BCV organisms comprising a polynucleotide at least about 80, 85, 90, 91, 92, 93, 94, 95, 96, 97, 98, 99, 99.5% or more identical to SEQ ID NO:5, SEQ ID NO:69, or SEQ ID NO:97.

### Example 5

### Identification and Classification of a Novel Picornavirus, Boone Cardiovirus

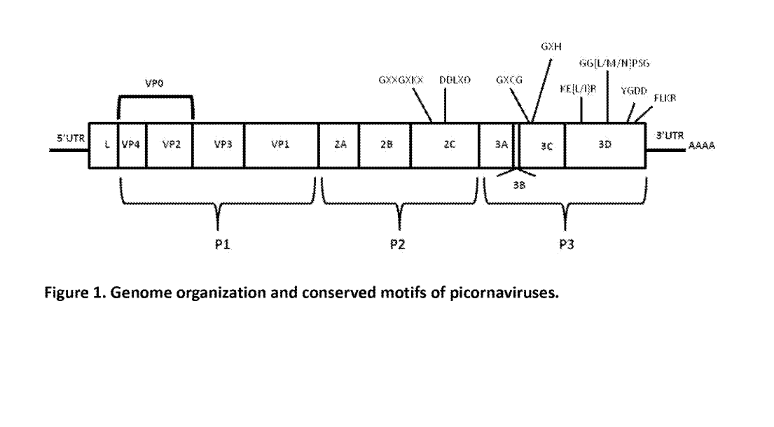

Feces from a colony of laboratory rats were screened for Picornaviruses by RT-PCR. One of the primer sets utilized, amplifies a conserved region of the 5′ UTR (20). With these primers, an approximately 200 nucleotide (nt) product was obtained and Blast analysis determined the product to be most similar to parechoviruses, a genus within the Picornaviridae family. However, when attempting to sequence the entire genome, degenerative primers designed to additional conserved regions of parechoviruses failed to generate sequencing products, suggesting that our rat virus was either divergent from known strains of parechoviruses or was not a parechovirus at all. Complete genome sequencing was accomplished by utilizing 5′ and 3′ RACE reactions. The entire viral genome was determined to be 8,504 nt, excluding the poly (A) tail. The sequence contains a 5′ UTR of 1,418 nt, an open reading frame of 6,944 nt, and a 3′ UTR of 140 nt. The genome has a 48% GC content, which is similar to cardioviruses, senecaviruses, and enteroviruses. This is a higher GC content than expected for hepatitis A, parechoviruses, and cosaviruses, and lower than expected for aphthoviruses, klasseviruses, and kobuviruses. The single open reading frame of the rat virus shared the typical organization of picornaviruses with the following predicted cleavage products; L, VP4, VP3, VP2, VP1, 2A, 2B, 2C, 3A, 3B, 3C, and 3D. FIG. 1. Not all picornaviruses encode a leader peptide preceding the P1, capsid region. Picornaviruses that encode leader peptides include those that belong to the generas of Cardiovirus, Aphthovirus, Erbovirus, Kobuvirus, Teschovirus, Seneca virus, and Sapelovirus. VP4, VP2, VP3, and VP1 are capsid proteins. 2A shuts off host protein synthesis. 2B and 2C are involved in membrane permeability and vesicle formation, 3AB is involved in initiation of RNA synthesis. 3C is a protease and 3D is a polymerase.

Within the non-structural proteins of picornaviruses there are several amino acid motifs commonly conserved amongst picornaviruses that were identified in the new rat virus. Two of these conserved motifs are located in the predicted 2C protein the NTPase motif GAPGQKS (aa 1309-1316) (SEQ ID NO:106), which is involved in NTPase binding and the helicase motif DDLGQ (aa 1358-1362) (SEQ ID NO:107). In the 3C protease protein the motif GXCG (aa 1788-1791) (SEQ ID NO:101), which is predicted to be a part of the protease active site and GXH (aa 1806-1808), the predicted site for substrate binding were also identified. Finally, in the 3D polymerase protein four motifs typically predicted to play a role in RNA template recognition and polymerase activity were present in the amino acid sequence (14, 15). These motifs include KDEIR (aa 2001-2005) (SEQ ID NO:102), GGLPSG (aa 2131-2136) (SEQ ID NO:103), YGDD (aa 2173-2176) (SEQ ID NO:104), and FLKR (aa 2221-2224) (SEQ ID NO:105).

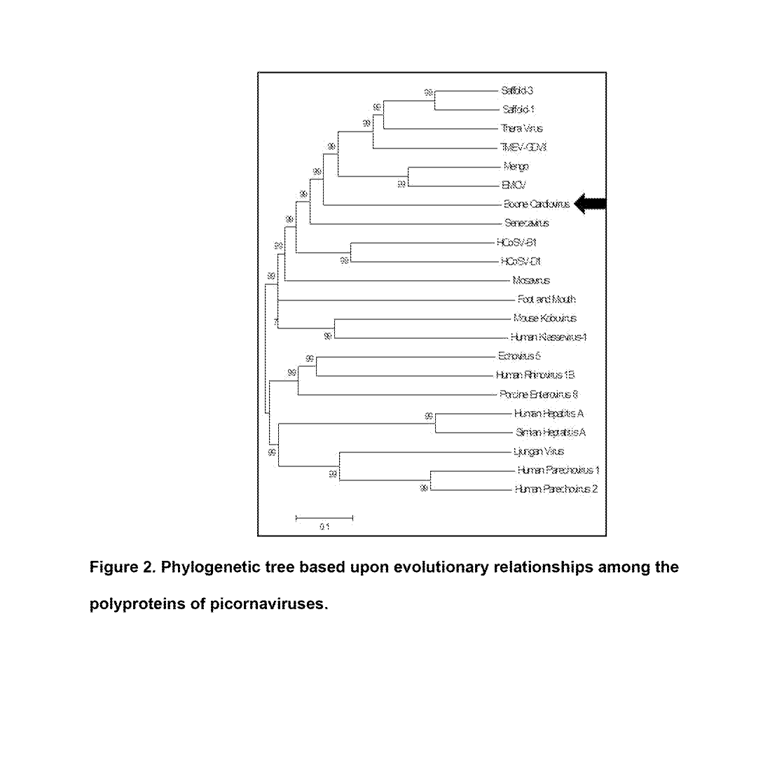

Nucleotide blast analysis of the entire genome showed the virus had greatest similarity to members of the Cardiovirus genera. The new viral genome was also aligned with representatives of the Picornaviridae family and a phylogenetic tree confirmed the closest relative to be the cardioviruses; however, the new virus did not cluster with either Theilovirus or EMCV species (FIG. 2). Based upon the ninth report from the International Committee on Taxonomy of Viruses (ICTV) the polyproteins of viruses belonging to different genera within the Picornaviridae family differ by at least 58% amino acid (aa) identity. The novel BCV virus differs from members of the Cardiovirus genera by 56-58%. BCV shares less than 45% amino acid identity in the polyprotein region with known strains of theiloviruses and EMCV and less than 50% amino acid identity in the P1 capsid protein. By these definitions this rat virus is on the borderline for the cardiovirus genus and we propose the name Boone Cardiovirus (BCV).

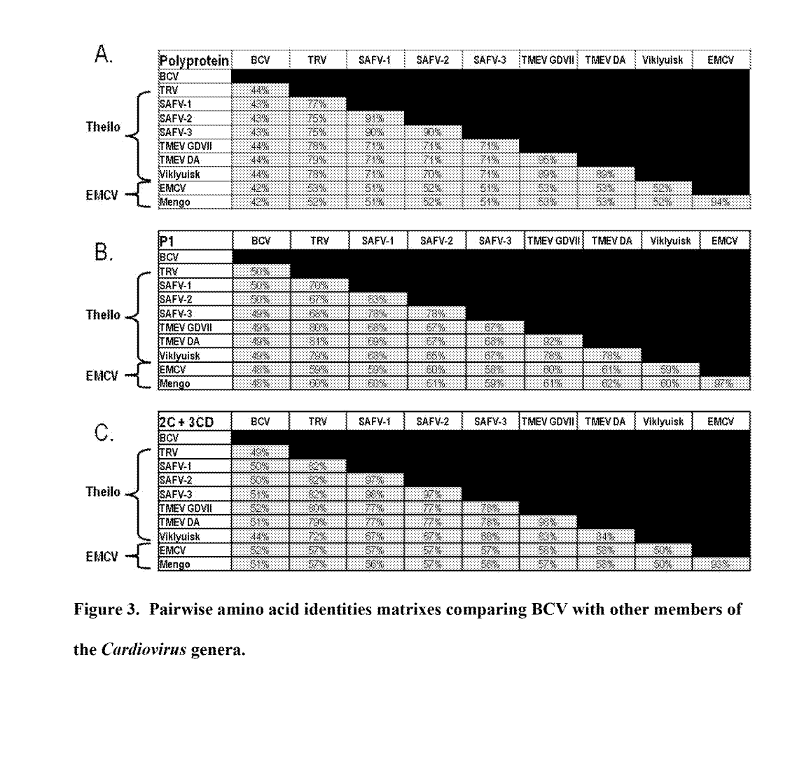

The ICTV also provides definitions for determining species within the cardiovirus genera. Currently, the only two identified species are Theiloviruses and EMCV. The definitions state that (1) a member of a species must share greater than 70% aa identity in the polyprotein, (2) share greater than 60% aa identity in the P1 region (VP4-VP1), (3) share greater than 70% aa identity in the 2C+3CD region, (4) share a natural host range, and (5) share a common genome organization. The polyprotein of BCV-1 shares only 43-44% and 42% aa identity with either theiloviruses or EMCV respectively (FIG. 3a). In the P1 region, BCV-1 shares only 47-48% aa identity with theiloviruses and 46-47% aa with EMCV (FIG. 3b). Within the 2C+3CD region of the genome BCV-1 shares 49-52% aa with the theiloviruses and 51-52% aa with EMCV (FIG. 3a). BCV does share a natural host with TRV and it shares the same common genome organization with all members of the cardiovirus genus, but according to the ICTV definitions BCV should be classified as a new species within the cardiovirus genera as it does not met the requirements within the polyprotein, P1, and 2C+3CD regions of the genome.

### Example 6

### Phylogenetic Analysis of the Leader Protein Coding Regions

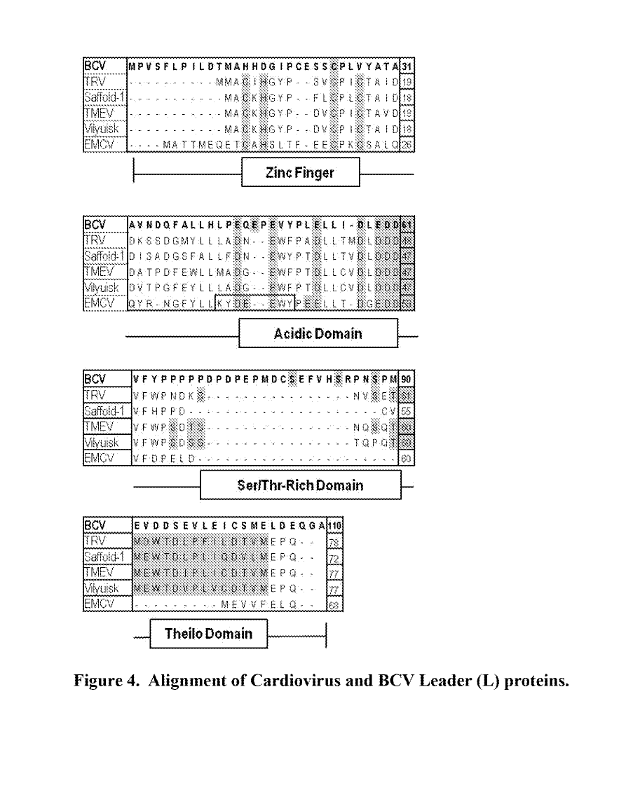

Within cardioviruses the leader (L) protein is the second most divergent protein, falling second to the L* protein. The leader protein of all known cardioviruses contains both a zinc finger motif (C-X-H-X(6)-C-X(2)-C) and an acidic domain. In TMEV and TRV, the leader protein also contains a Ser/Thr-rich domain. This Ser/Thr-rich domain is partially deleted in SAFV strains and is completely deleted in EMCV. Interestingly, BCV does not contain a zinc finger domain within its leader protein, but it does encode both an acidic domain and a Ser/Thr-rich domain (FIG. 4). In strains of EMCV, the acidic domain contains a threonine residue that has been predicted to become phosphorylated as it is part of a tyrosine kinase phosphorylation domain, [KR]-X(2,3)-[ED]-X(2,3)-Y (31). This potential phosphorylation site has also been predicted to exist in SAFV; however, BCV lacks a threonine residue within the acidic domain as well as the predicted tyrosine kinase phosphorylation domain. At the C′ terminal end of the leader protein there is a conserved region found among strains of TMEV, RTV, and SAVF that is lacking in strains of EMCV; as a result, this domain has been named the theilo domain (32). The BCV leader protein does not encode the theilo domain.

### Example 7

### Phylogenetic Analysis of the L* Protein Coding Regions

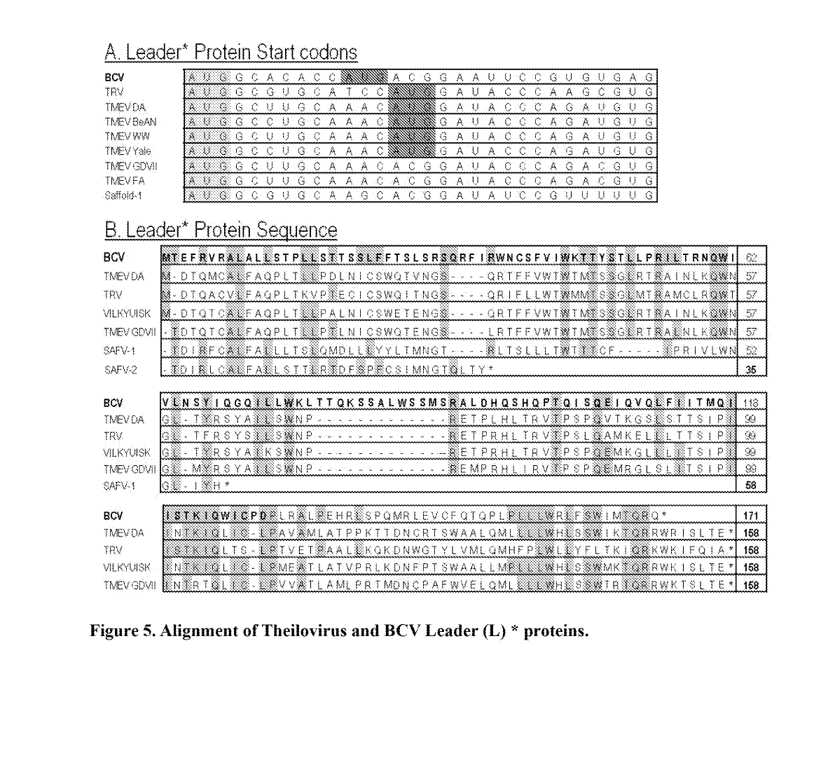

The L* protein is produced by only a subset of the cardioviruses and is translated from an alternative start codon downstream of the polyprotein's AUG initiation sequence (FIG. 5a). In TMEV TO strains (DA, WW, BeAN, and Yale) the L* protein has been reported to play a role in persistence and demyelination (8). BCV contains an AUG start codon in frame with the AUG start codon of TO strains of TMEV. If functional, the BCV L* protein is roughly 20 aa longer than the L* protein produced by TMEV TO strains, 171 aa compared to 156 aa (FIG. 5b). Highly neurovirulent strains of TMEV (GDVII and FA), SAFV and EMCV are not predicted to encode a functional L* protein due to an ACG rather than AUG start codon.

### Example 8

### Phylogenetic Analysis of the BCV Polyprotein

The complete genome of BCV-1 was aligned with representatives from all species of cardioviruses and a similarity plot was generated to visually compare the genomes at the nucleotide level (FIG. 6). The plot reiterates that BCV is divergent from both EMCV and Theiloviruses. Analysis of the BCV genome reveals that regions within the capsid proteins, VP1-VP3, have some of the highest degree of nucleotide identity with other cardioviruses.

It is known that capsid proteins VP1 and VP2 of cardioviruses contain four neutralizing immunogenic sites that can affect a strain's virulence. Strains of TMEV show very little variability within these regions and a high degree of conservation is seen amongst VHEV, TRV, and TMEV. SAFV and EMCV strains on the other hand, share very little conservation with the other cardioviruses.

The VP1 encodes two antigenic sites known as the CD loops I and II. Within CD loop I, BCV has no amino acid identity with any of the cardioviruses and the region is mostly deleted (FIG. 7a). In the BCV CD loop II only a few amino acids are shared with those of other cardioviruses and CD loop I is partially deleted. The two neutralizing antigen sites in the VP2 protein are referred to as EF loops I and II. In BCV, EF loop I is deleted and EF loop II of BCV shares the greatest homology with SAFV, 26% aa identity (5/19) (FIG. 7b). In addition, to containing EF neutralizing sites, three amino acids within VP2 of TO TMEV strains have been shown to act as co-receptors on the surface of the virus by binding α2,3 N-linked sialic acid residues (30). These residues are not present in BCV.

### Example 9

Any suitable primers can be used to specifically and sensitively amplify parts of the BCV genome from, e.g., the feces or tissues of infected rodents. For example, primers and PCR assays that target the virus sequence from about nucleotide 1452 to about nucleotide 8363 (e.g., about 6166 to about 6570) can be used. These assays are sensitive, able to detect as few as 1-10 genomic copies, and specific for amplification of BCV. For example, PCR forward primer SEQ ID NO:108 and reverse primer SEQ ID NO:109 can be used to amplify a product of 119 nucleotides.

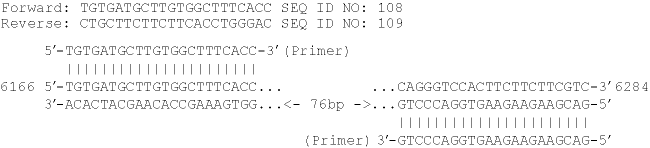

The one-step RT-PCR parameters for this reaction are as follows:

| | |
|---|---|
| Reverse Transcription | 50° c. for 30 minutes |
| Inactivation | 95° c. for 15 minutes |
| *Denature | 94° c. for 30 seconds |
| *Anneal | 56° c. for 30 seconds |
| *Extend | 72° c. for 30 seconds |
| Final Extension | 72° c. for 07 minutes |

*Temperatures are repeated for a total of 40 cycles

Another set of primers that can be used to specifically and sensitively amplify BCV are:

SEQ ID NO: 110
Forward: AGAAGCCCCAGCAATGTCCCCAG
SEQ ID NO: 111
Reverse: CCGCCCTTGCAAATTGCCTGAATG.

These primers amplify a 234 nucleotide product.

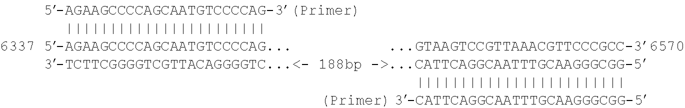

The one-step RT-PCR parameters for this reaction are as follows:

| | |
|---|---|
| Reverse Transcription | 50° c. for 30 minutes |
| Inactivation | 95° c. for 15 minutes |
| *Denature | 94° c. for 30 seconds |
| *Anneal | 60° c. for 30 seconds |
| *Extend | 72° c. for 30 seconds |
| Final Extension | 72° c. for 07 minutes |

*Temperatures are repeated for a total of 40 cycles

### Discussion

A novel picornavirus, Boone Cardiovirus (BCV) was isolated from the feces of asymptomatic laboratory rats. Initial sequence analysis suggested the virus belonged in the Picornaviridae family due to several conserved picornavirus elements (FIG. 1). Based upon GC content and prediction of both a leader protein, BCV was predicted to be most closely related to either the Cardiovirus or Senecavirus genera. This classification was confirmed by further phylogenetic analysis that showed BCV is a new species of cardiovirus that is equally divergent from both EMCV and Theilovirus species. The ICTV definitions for cardiovirus species determination state that a member of a species must share greater than 70% aa identity in the polyprotein, greater than 60% aa identity in the P1 region, greater than 70% aa identity in the 2C+3CD region, share a natural host range, and a common genome organization. BCV when compared to either EMCV or Theiloviruses satisfies only two of the five requirements and as a result should be considered a novel species within the cardiovirus genus.

Phylogenetic analysis determined that BCV encodes an L protein that shares only some of the typical characteristics of other cardioviruses. Leader proteins have been identified in several picornaviruses such as Cardioviruses, Aphthoviruses, Erboviruses, Kobuviruses, Teschoviruses, and Sapeloviruses. The leader proteins of aphtho- and erbo-viruses act as a papain-like cysteine proteinase that cleave eukaryotic initiation factors, resulting in the shut off of host protein synthesis. In cardioviruses, the L protein is believed to play a critical role in cytosol-dependent phosphorylation cascades involved in nucleocytoplasmic trafficking and cytokine expression (6, 7, 24).

Two distinguishing features of cardiovirus L proteins were identified in BCV, the acidic and thr/ser domains. The ser/thr domain is found in both TMEV and TRV species of Theiloviruses, but is partially deleted in SAFV strains and completely deleted in EMCV. The most unique feature of the BCV L protein as compared to other cardioviruses is the lack of an identifiable zinc finger, which has been identified in all other species. Historically, when the zinc finger motif was removed from TMEV in vitro, apoptosis of infected cells was not observed (3, 7). Apoptosis is a method of viral spread during infection and this deficiency can attenuate viral infections. Dvorak et al. observed that deletion of the zinc finger motif in EMCV led to restricted infections and reduce protein synthesis (6). BCV has not been propagated in cell culture despite attempts in over fifteen different cell lines and varied growth conditions. Whether the lack of a zinc finger motif in the L protein can contribute to these difficulties has yet to be determined. In vivo, zinc finger mutations reduced viral titers of persistent TMEV in the spinal cords of mice (25). Mutations in the zinc finger motif have also shown to decrease the anti-alpha/beta interferon responses during viral infections (3, 4, 7, 29).

Despite the evidence that zinc fingers in the leader protein play an important role in cardiovirus infections, evidence suggests that the domains of the L protein act synergistically. Ricour et al. generated independent mutations in the zinc finger and theilodomains and showed that these mutations affected all of the L protein functions that were tested including nucleocytoplasmic trafficking and interferon responses (24). This is further supported by the fact that the EMCV L protein does not encode the theilo or ser/thr domains; however, it has retained the ability to modulate the same processes as theiloviruses (22). More recently discovered picornaviruses, such as mouse kobuvirus and senecavirus also encode cardiovirus-like L proteins, but lack the zinc finger motif similar to BCV (10, 23).

Laboratory rats can be persistently infected with BCV. By RT-PCR continual fecal shedding from naturally infected rats 5 weeks to 10 months of age can be detected. In TO strains of TMEV the L* protein plays a crucial role in viral growth in macrophages and persistence infections of the host (26, 27). Analysis of the BCV genome predicts that like the TO TMEV strains it produces a functional L* protein. A second characteristic of TO TMEV strains that has been shown to be associated with persistence is the use of sialic acid as a co-receptor for viral entry. Three amino acids (FIG. 7b) of the VP2 protein have been identified as playing a direct role in the binding of sialic acid (16, 30). These amino acids are conserved in non-persistent TMEV strains; however, it has been suggested that the overall protein structure inhibits sialic acid binding. These amino acids are not conserved by BCV. In the case of BCV, it is more likely that persistence is encoded by the L* protein or by another unidentified genomic element than, by the binding of sialic acid.

Cardioviruses have exposed surfaces on their capsids that are involved in host cell tropism and act as immunogenic sites that can affect virulence. These sites are the CD and EF loops located within the VP1 and VP2 proteins respectively. Despite the fact, that some regions of highest shared amino acid identity between BCV and cardioviruses are found in these capsid regions, BCV shares very little amino acid identity in either of the CD and EF loops (FIG. 7). This indicates that the exposed surface of BCV mostly likely has a unique secondary structure as compared to known cardioviruses and suggests that BCV has the potential to enter cells through a different host receptor.

BCV is a seemingly non-pathogenic virus as infected rats do not present with clinical symptoms. Despite appearing non-pathogenic due to the persistent nature of BCV infections the long term consequences of infection and should be evaluated. Understanding ostensibly mild viruses can be just as useful as understanding those that are pathogenic with clear clinical presentations. Understanding BCV infection may play useful in further understanding the difference between aspects of the cardiovirus genome that contribute to clinical symptoms in both rodents and humans and the regions that do not. Most likely BCV does not go undetected by the host immune system and understanding how the virus is kept in check may hold clues to identifying novel antivirals for the pathogenic strains of cardioviruses and other picornaviruses. BCV may also prove useful as a comparative strain for understanding the many “orphan” viruses that have recently been discovered that have cardiovirus elements, but which relatively little is known.

### REFERENCES

1. Abed & Boivin. 2008. Emerg Infect Dis 14:834-6.
2. Blinkova et al., 2009. J Virol 83:4631-41.
3. Chen et al., 1995. J Virol 69:8076-8.
4. Delhaye et al., 2004. J Virol 78:4357-62.
5. Drake et al., 2008. Comp Med 58:458-64.
6. Dvorak et al., 2001. Virology 290:261-71.
7. Fan et al., 2009. J Virol 83:6546-53.
8. Ghadge et al., 1998. J Virol 72:8605-12.
9. Goldfarb & Gajdusek. 1992. Brain 115 (Pt 4):961-78.
10. Hales et al., 2008. J Gen Virol 89:1265-75.
11. Hall, 1999. Nucl. Acids. Symp. Ser 41:95-98.
12. Himeda & Ohara. 2012. J Virol 86:1292-6.
13. International Committee on Taxonomy of Viruses., and A. M. Q. King. 2012. Virus taxonomy: classification and nomenclature of viruses: ninth report of the International Committee on Taxonomy of Viruses. Academic Press, London; Waltham, Mass.
14. Jablonski & Morrow. 1993. J Virol 67:373-81.
15. Jablonski & Morrow. 1995. J Virol 69:1532-9.
16. Kumar et al., 2003. J Virol 77:2709-16.
17. Lole et al., 1999. J Virol 73:152-60.
18. Lorch et al., 1981. J Virol 40:560-7.
19. Nielsen et al., 2012. Emerg Infect Dis 18:7-12.
20. Nix et al., 2008. J Clin Microbiol 46:2519-24.
21. Ohsawa et al., 2003. Comp Med 53:191-6.
22. Paul & Michiels. 2006. J Gen Virol 87:1237-46.
23. Phan et al., 2011. PLoS Pathog 7:e1002218.
24. Ricour et al., 2009. J Virol 83:11223-32.
25. Sallie, 1993. PCR Methods Appl 3:54-6.
26. Takano-Maruyama et al., 2006. J Neuroinflammation 3:19.
27. Takata et al., 1998. J Virol 72:4950-5.
28. Tamura et al., 2011. Mol Biol Evol 28:2731-9.
29. van Pesch et al., 2001. J Virol 75:7811-7.
30. Zhou et al., 1997. J Virol 71:9701-12.
31. Zoll et al., 2002. J Virol 76:9664-72.
32. Ricour et al., 2009. J Virol 83:11223-32.
33. Devaney et al., 1988. J Virol 62:4407-9.
34. Gorbalenya et al., 1991. FEBS Lett 288:201-5.

We claim:

1. An isolated polynucleotide molecule comprising: (a) SEQ ID NO:97; (b) a polynucleotide at least about 80%, 85%, 90%, 95%, 98% or more homologous to SEQ ID NO:97; (c) a polynucleotide comprising at least about 20 contiguous nucleic acids of SEQ ID NO:97; (d) GenBank accession number JQ864242 or JX683808; or (e) a complement of (a), (b), (c), or (d).

2. The isolated polynucleotide of claim 1, wherein the polynucleotide is SEQ ID NO:5, 42-56, 69-83 or a polynucleotide comprising about 20 or more contiguous nucleic acids of SEQ ID NO:5, 42-56, 69-83, or a complement thereof.

3. A substantially purified polypeptide encoded by the polynucleotide of claim 1.

4. (canceled)

5. An isolated antibody or antigen binding fragment thereof that specifically binds to the substantially purified polypeptide of claim 3.

6. (canceled)

7. An expression vector or host cell comprising an expression vector, wherein the expression vector comprises the isolated polynucleotide of claim 1 or a fragment thereof.

8. A method of determining the presence or absence of Boone cardiovirus polynucleotides, polypeptides, or antibodies or specific binding fragments thereof that specifically bind to a Boone cardiovirus polypeptide comprising:
(a) obtaining a test sample; and
(b) determining the presence or absence of Boone Cardiovirus polynucleotides, polypeptides, or antibodies or specific binding fragments thereof in the test sample.

9. The method of claim 8, wherein the Boone cardiovirus has a genome of GenBank accession number JQ864242 or JX683808, or a genome that is 85%, 90%, 95%, or 98% identical to GenBank accession number JQ864242 or JX683808, that is 85%, 90%, 95%, or 98% identical to SEQ ID NO:97, or a complement thereof.

10. The method of claim 8, wherein the test sample is from a mammal that is subject to potential infection by Boone cardiovirus.

11. The method of claim 8, comprising a method of detecting a Boone cardiovirus polynucleotide comprising: a) amplifying polynucleotides of the test sample with at least one primer that hybridizes to at least 10 contiguous nucleic acids of SEQ ID NO:97, or a complement thereof, to produce an amplification product; and b) detecting the presence of the amplification product, thereby detecting the presence of the Boone cardiovirus polynucleotide.

12. The method of claim 11, wherein the method comprises the use of at least two primers selected from (a) SEQ ID NO:108 and 109 or (b) SEQ ID NO:6 and SEQ ID NO:7.

13. The method of claim 11, wherein the polynucleotides are amplified using a method selected from the group consisting of transcription mediated amplification (TMA), polymerase chain reaction (PCR), reverse-transcriptase PCR (RT-PCR), quantitative PCR, replicase mediated amplification, ligase chain reaction (LCR), competitive quantitative PCR (QPCR), real-time quantitative PCR, self-sustained sequence replication, strand displacement amplification, branched DNA signal amplification, nested PCR, in situ hybridization, multiplex PCR, Rolling Circle Amplification (RCA), and Q-beta-replicase system.

14. The method of claim 11, wherein the quantity of amplification products is determined.

15. The method of claim 8, comprising a method of detecting the presence of Boone cardiovirus polynucleotides in the test sample comprising: contacting the sample with one or more isolated nucleic acid probes comprising about 10 or more contiguous nucleic acids of SEQ ID NO:97; and detecting the presence of hybridized probe/Boone cardiovirus nucleic acid complexes, wherein the presence of hybridized probe/Boone cardiovirus nucleic acid complexes indicates the presence of Boone cardiovirus in the test sample.

16. The method of claim 8, comprising a method of detecting Boone cardiovirus polypeptides in the test sample comprising: a) contacting the test sample with an isolated antibody or antigen binding fragment thereof that specifically binds to a substantially purified polypeptide encoded by a polynucleotide molecule comprising (i) SEQ ID NO:97; (ii) a polynucleotide at least about 80%, 85%, 90%, 95%, 98% or more homologous to SEQ ID NO:97; (iii) a polynucleotide comprising at least about 20 contiguous nucleic acids of SEQ ID NO:97; (iv) GenBank accession number JQ864242 or JX683808; or (v) a complement of (i), (ii), (iii), or (iv) to form Boone cardiovirus polypeptide/antibody complexes; and b) detecting the presence of the Boone cardiovirus polypeptide/antibody complexes, thereby detecting the presence of the Boone cardiovirus polypeptides.

17. The method of claim 16, wherein the polypeptide/antibody complexes are detected by a technique comprising enzyme-linked immunosorbent assay (ELISA), multiplex fluorescent immunoassay (MFI or MFIA), radioimmunoassay (RIA), sandwich assay, Western blotting, immunoblotting analysis, an immunohistochemistry method, immunofluorescence assay, or a combination thereof.

18. The method of claim 8 comprising a method of detecting antibodies that specifically bind a Boone cardiovirus polypeptide in the test sample, comprising:
(a) contacting one or more of a purified polypeptides encoded by a polynucleotide molecule comprising (i) SEQ ID NO:97; (ii) a polynucleotide at least about 80%, 85%, 90%, 95%, 98% or more homologous to SEQ ID NO:97; (iii) a polynucleotide comprising at least about 20 contiguous nucleic acids of SEQ ID NO:97; (iv) GenBank accession number JQ864242 or JX683808; or (v) a complement of (i), (ii), (iii), or (iv) with the test sample, under conditions that allow polypeptide/antibody complexes to form; and
(b) detecting the polypeptide/antibody complexes;
wherein the detection of the polypeptide/antibody complexes is an indication that antibodies specific for a Boone cardiovirus polypeptide are present in the test sample.

19. The method of claim 19, wherein the polypeptide/antibody complexes are detected by a technique comprising enzyme-linked immunosorbent assay (ELISA), multiplex fluorescent immunoassay (MFI or MFIA), radioimmunoassay (RIA), sandwich assay, Western blotting, immunoblotting analysis, an immunohistochemistry method, immunofluorescence assay, or a combination thereof.

20. A kit for detecting a Boone cardiovirus polynucleotides or polypeptides comprising at least one of: (a) SEQ ID NO:97; (b) one or more polynucleotides at least about 80%, 85%, 90%, 95%, 98% or more homologous to SEQ ID NO:97; (c) one or more polynucleotides comprising at least about 20 contiguous nucleic acids of SEQ ID NO:97; (d) one or more complements of (a), (b) or (c); (e) one or more substantially purified polypeptides encoded by the one or more polynucleotides of (a), (b), (c), or (d); (f) one or more isolated antibodies or antigen binding fragments thereof that specifically bind to the substantially purified polypeptides of (e); or (g) combinations thereof.

21. The kit of claim 21 further comprising one or more polynucleotides, one or more substantially purified polypeptides, one or more antibodies or antigen binding fragments that can detect one or more viruses, bacteria, fungi or protozoans other than Boone cardiovirus.
